SEQUENCE LISTING (1 sequence)

SEQ ID NO: 1 - protein, 324 aa, Terribacillus saccharophilus [sequence omitted]

(19) United States
(12) Patent Application Publication
(10) Pub. No.: US 2026/0250606 A1
(43) Pub. Date: Aug. 27, 2026

(54) WASHING AND CLEANING AGENTS COMPRISING DISPERSIN AND FRAGRANCE

(71) Applicant: Henkel AG & Co KGaA, Düsseldorf (DE)

(72) Inventors: Mirko Weide, Düsseldorf (DE); Michael Kandzia, Solingen (DE); Frank Janssen, Hagen (DE); Thomas Wilke, Monheim (DE); Sheila Edwards, Düsseldorf (DE); Justine Felder, Monchengladbach (DE); Susanne Schneider, Düsseldorf (DE); Inga Kerstin Vockenroth, Düsseldorf (DE); Anika Velleuer, Düsseldorf (DE); Detlef Buisker, Essen (DE)

(21) Appl. No.: 19/159,406

(22) PCT Filed: Jan. 25, 2024
(86) PCT No.: PCT/EP2024/051751

§ 371 Date: Aug. 25, 2025

(30) Foreign Application Priority Data
Feb. 24, 2023 (DE) 102023201692.1

(51) Int. Cl. C11D 3/50; C11D 1/66; C11D 1/90; C11D 3/04; C11D 3/10; C11D 3/22; C11D 3/33; C11D 3/36; C11D 3/37; C11D 3/382; C11D 3/386; C11D 3/43; C11D 17/00; C11D 17/08

(52) U.S. Cl. CPC C11D 3/50; C11D 1/66; C11D 1/90; C11D 3/042; C11D 3/10; C11D 3/225; C11D 3/33; C11D 3/361; C11D 3/3746; C11D 3/382; C11D 3/386; C11D 3/43; C11D 17/0065; C11D 17/08; C11D 2111/12; C11D 2111/14

(57) ABSTRACT

The invention lies in the field of washing and cleaning agents, in particular enzyme-containing washing and cleaning agents. The invention relates to washing and/or cleaning agents, preferably textile washing agents, preferably pre-portioned textile washing agents, comprising (A) at least one dispersin, the dispersin exhibiting β-1,6-N-acetylglucosaminidase activity and comprising an amino acid sequence which is at least 70% identical to the amino acid sequence specified in SEQ ID NO:1 over its entire length and has at least one amino acid substitution and/or deletion at specific positions, (B) at least one fragrance, and (C) at least one ingredient. The invention also relates to the corresponding washing and/or cleaning methods, the use of the agents described herein, and the use of dispersin to reduce bad odors in the washed textiles and to increase the fragrance intensity of the fragrance in the washing and/or cleaning agent.

The invention lies in the field of washing and cleaning agents, in particular enzyme- and fragrance-containing washing and cleaning agents. The invention relates to a washing and/or cleaning agent, in particular a textile washing agent, particularly preferably a pre-portioned textile washing agent, comprising at least one dispersin and at least one fragrance. The invention also relates to the corresponding washing and/or cleaning methods and the use of the agents described herein for cleaning textiles and/or hard surfaces, in particular dishes. Furthermore, the invention relates to the use of a dispersin in a washing and/or cleaning agent for reducing the malodor of a textile or a surface that has been treated with the washing and/or cleaning agent, and to the use of a dispersin for increasing the fragrance intensity of the fragrance contained in the washing and/or cleaning agent on a textile or a surface that has been treated with the washing and/or cleaning agent.

The use of enzymes in washing agents has been established in the prior art for decades. They are used to expand the performance range of the agents in question according to their special activities. These include in particular hydrolytic enzymes such as proteases, amylases, lipases and cellulases. The first three mentioned hydrolyze proteins, starch and fats and thus contribute directly to the removal of dirt. Cellulases are used in textile washing agents in particular due to their effect on fabric. Another group of washing agent enzymes are oxidative enzymes, in particular oxidases, which, optionally in conjunction with other components, are preferably used to bleach stains and/or to produce the bleaching agents in situ. In addition to these enzymes, which are subject to continuous optimization, other enzymes are constantly being made available for use in washing agents so that, in particular, specific stains can be optimally tackled, such as pectinases, pectate lyases, xanthanases, β-glucanases, mannanases, dispersins and the like, for hydrolyzing specific plant polymers in particular. In general, only selected enzymes are suitable for use in liquid surfactant-containing preparations. Many enzymes do not exhibit sufficient catalytic performance in such preparations. For the use of enzymes in cleaning agents, therefore, a high catalytic activity under conditions as they are during a wash cycle and a high storage stability is particularly desirable.

Continuously changing requirements are placed on the forms of manufacture and supply of washing and cleaning agents. The main focus has, for quite some time, been on the convenient dosing of washing and cleaning agents by the consumer and the simplification of the work steps necessary for carrying out a washing or cleaning method. A technical solution is provided by pre-portioned washing or cleaning agents, for example film bags comprising one or more receiving chambers for solid and/or liquid washing and cleaning agents. A trend relevant to the production of these film pouches is the miniaturization of these film pouches. In addition to higher consumer acceptance due to simplified handling, the background of this development is, in particular, sustainability aspects, for example in relation to transport volumes and costs and the quantity of packaging materials used. The concentration of modem washing and cleaning agents presupposes the progressive optimization of existing formulations and the development of new, more efficient formulations. One aim of these developments, in which generally known active washing and cleaning ingredients are used, is to achieve an increase in the washing or cleaning performance of the formulations with a constant or reduced dosing quantity.

The most important criterion in cleaning textiles and/or hard surfaces is the cleaning performance on a wide variety of stains. Even though the cleaning performance of the washing and cleaning agents used today is generally high, the general trend towards the increased use of low-temperature programs and the trend towards compacting formulations (in particular for pre-portioned forms of use) has led to the problem that many of the usual washing and cleaning agent formulations have insufficient cleaning performance, in particular on stubborn stains. Such an inadequate cleaning performance leads to dissatisfaction among consumers and to the fact that such stains are pretreated by consumers, which in turn increases the consumption of water and energy.

The term “malodor” as used herein means an odor that is undesirable on surfaces, in particular household surfaces, dishes, textiles and cleaned objects, in particular cleaned textiles and cleaned dishes. The cleaned object should smell fresh and clean without any malodor clinging to it. A wide range of causes can result in an unpleasant odor, so-called malodor, on surfaces, in particular household surfaces, and on washed textiles and/or cleaned hard surfaces, in particular dishes. The term “household surface” as used herein means, for example, surfaces in washing machines or dishwashers, pump sumps, storage tanks, water reservoirs, washing drums, rinsing chambers, washbasins or sinks, toilet bowls, flush tanks, toilet rims, siphons, water pipes and/or lines, water inlets and/or outlets. Malodor is a known problem in particular with respect to textiles and washing machines. But malodors can also be found in the dishwasher. This affects all components that come into contact with dirty objects and/or water, in particular used water, and/or washing/cleaning suds, such as surfaces in washing machines or dishwashers, pump sumps, storage tanks, water reservoirs, washing drums, rinsing chambers, washbasins or sinks, siphons, water pipes and/or lines, water inlets and/or outlets. One, if not the most common, cause of this are organic in nature, such as sebum, body soils (e.g., dead skin cells and sweat), cell residues, food residues, body excretions, biofilms and extracellular polymeric substances (EPS; e.g., extracellular polymeric saccharides). In particular, microbially induced bio-buildups, i.e., microbially induced malodor as a result of microbially induced bio-buildups, should be emphasized here. Another example of unpleasant odors is sweat or body odor that clings to an object that has come into contact with people or animals. Malodor is found not only in the washing machine but also on freshly washed, still damp laundry, and on dry laundry, and on numerous other surfaces, in particular household surfaces and dishes. This also includes so-called rebloom, i.e., the revival of the malodor when the laundry is worn, due to body heat and/or body moisture. However, deposits, bio-buildups, body soils and the like not only lead to malodor but can also become visible as stains on textiles and/or surfaces, in particular household surfaces and dishes, e.g., in the form of buildups, smears, encrustations, yellowing, armpit stains and graying. Due to their typically adhesive texture, bio-buildups and the like also allow other malodor particles and/or dirt particles to adhere. However, malodor can also be present before the microbially induced deposits lead to visible stains.

The term “bio-buildup” as used herein means not only biofilms in the narrower sense, such as microbially induced deposits, biofilms or the like, which can be formed by any microorganisms whose cells adhere to each other and/or to a surface, e.g., to textiles, dishes or a hard surface or another type of surface, but also more generally any bio-buildups, biosoils, biologically induced buildups or the like including, but not limited to, waste products and secretions of microorganisms that adhere to a surface, e.g., to textiles, dishes or a hard surface or another type of surface. These adherent cells are often embedded in a self-produced matrix of extracellular polymeric substance (EPS). Biofilm EPS is a polymeric conglomerate generally comprising extracellular DNA, proteins and polysaccharides. Biofilms can form on living or non-living surfaces. The microbial cells that grow in a biofilm are physiologically different from the planktonic cells of the same organism, which, in contrast, are single cells that float or can float in a liquid medium. Bacterial cells living in a biofilm usually have significantly different properties than planktonic bacterial cells of the same species, because the dense and protected environment of the film allows them to cooperate and interact in a different way. One advantage of this environment for the microorganisms is the increased resistance to washing agents and antibiotics, because the dense extracellular matrix and the outer cell layer protect the inside of the community. Biofilm-producing bacteria on textiles include, for example, the following species: Acinetobacter sp., Aeromicrobium sp., Brevundimonas sp., Microbacterium sp., Micrococcus luteus, Pseudomonas sp., Staphylococcus epidermidis and Stenotrophomonas sp. On hard surfaces, biofilm-forming bacteria can be found, for example, among the following species: Acinetobacter sp., Aeromicrobium sp., Brevundimonas sp., Microbacterium sp., Micrococcus luteus, Pseudomonas sp, Staphylococcus epidermidis, Staphylococcus aureus and Stenotrophomonas sp.

EP 3892708, WO 2021/122117, WO 2021/122118, WO 2021/122120, WO 2021/122121 and WO 2022/008387 describe dispersin- and enzyme-containing washing and cleaning agents. It has also been described that dispersin can decompose biofilm and EPS, for example.

Washing agents and cleaning agents that contain one or more fragrances are well known. Fragrances often have a dual function and are intended not only to create a desired scent, e.g., pleasant smell, but also to mask undesirable odors, e.g., malodor. The fragrances can be, for example, chemical compounds, synthetic compounds or natural oils such as essential oils and other natural compounds. WO 2011/163325 describes various perfume raw materials, perfume delivery systems and consumer products containing such perfume raw materials and/or such perfume delivery systems.

Accumulated organic soiling is often difficult to remove, and commercially available washing and cleaning agents do not sufficiently reduce the malodor. Instead, the malodor is masked by the use of various fragrances, which is often perceived by consumers as a cleaning effect, although the causes of the malodors are not actually eliminated. As a result, more and more organic material and therefore more and more malodors accumulate over time and more and more fragrances have to be added to prevent the textiles from smelling worse and worse. However, many fragrances have side effects such as allergenic properties, which can manifest themselves in the form of skin rashes. In addition, many of these substances, as natural chemicals, are harmful to the environment if they get into wastewater. In recent years, fragrances have increasingly been used in the form of fragrance microcapsules, although it is known that microcapsules are considered microplastics and are therefore potentially harmful to the environment. Fragrances are also expensive and add to the overall cost of washing and cleaning products.

There is therefore a need to further improve the cleaning performance of enzyme-containing washing and cleaning agents, in particular of pre-portioned textile washing agents, in particular with regard to stubborn stains. There is therefore a further need to reduce the malodor in washing machines and dishwashers and/or on the washed textiles or cleaned surfaces, particularly dishes.

Surprisingly, it has now been found that the dispersin according to the invention not only combats the cause of malodor, in particular malodor caused by bio-buildup and/or body soil, but also interacts synergistically with the at least one fragrance contained in the washing and cleaning agent. The synergistic interaction of dispersin and fragrance not only leads to a reduction in malodors, but also to a freshness boost and increased fragrance intensity. Furthermore, it has surprisingly been found that this effect, in particular the increased fragrance intensity, also occurs with a reduced amount of fragrance (compared to a conventional washing and cleaning agent), wherein this effect is present both after washing and during wear, e.g., after the first sweating, and on the dry laundry.

Furthermore, it has surprisingly been found that the dispersin according to the invention contributes to the deep clean effect, i.e., pore-deep cleaning, of textiles and/or surfaces, in particular dishes. The deep clean effect not only includes pore-deep purity in terms of soiling, but also the reduction or elimination of malodors, as well as increased hygiene of the treated textiles and/or surfaces, since the removal of bio-buildups also removes microbes adhering to them, e.g., bacteria. The term “deep cleaning”, as used herein, refers in particular to the dissolution or removal of components of organic substances, e.g., sebum, cell residues, body soil, bio-buildups, biofilms (including, e.g., polysaccharides, PNAG (poly-N-acetyl-glucosamine)), body oils or other components contained in organic substances.

In a first aspect, the invention therefore relates to a washing and/or cleaning agent, preferably a textile washing agent, preferably a pre-portioned textile washing agent, comprising
(A) at least one dispersin, wherein the dispersin exhibits β-1,6-N-acetylglucosaminidase activity and comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion, preferably in an amount of active protein of 0.00001 to 5 wt. %, preferably 0.0003 to 0.5 wt. %, more preferably 0.0005 to 0.25 wt. %, particularly preferably 0.001 to 0.25 wt. %, based on the total weight of the agent;
(B) at least one fragrance, preferably in an amount of 0.01 to 5 wt. %, preferably 0.05 to 3 wt. %, particularly preferably 0.1 to 1 wt. %, based on the total weight of the agent, and
(C) at least one ingredient, preferably in an amount of 0.01 to 99.9 wt. %, based on the total weight of the agent.

In a further aspect, the invention relates to a washing and/or cleaning agent, preferably a textile washing agent, particularly preferably a pre-portioned textile washing agent, comprising
(A) at least one dispersin, wherein the dispersin exhibits β-1,6-N-acetylglucosaminidase activity, comprises an amino acid sequence which is at least 70% and increasingly preferably at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the amino acid sequence indicated in SEQ ID NO:1 and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion selected from the group consisting of D2A, D2L, D2N, D2R, D2V, D2W, Q3F, Q3I, Q3L, Q3M, Q3P, Q3V, Q3Y, Q3T, S12A, H15F, H15Y, T17W, T17C, T17E, T17F, T17M, T17R, T17V, V18L, E19D, E19N, E19P, K22A, K22M, K22V, S23C, S23E, S23I, S23L, S23R, S23T, S23V, V25R, D26M, Y30*, Y30D, Y30L, Y30M, Y30N, Y30R, Y30T, Y30V, G32L, G32M, G32R, N43*, N43H, N43L, E44*, N45D, N45L, N45V, A49W, A49Y, Y52*, Y52M, G54L, G54M, G54N, S56T, S56W, S57W, E58N, N59A, N59C, N59D, N59E, N59F, N59M, N59R, N59V, N59W, T60V, N62C, N62D, N62H, N62Q, N62W, T63C, T63D, T63L, T63N, T63R, T63V, K67A, K67L, N68L, N68Q, L71H, L71N, L71R, L71V, L71W, S72*, S72C, S72D, S72E, S72F, S72G, S72I, S72M, S72N, S72R, S72T, S72Y, I74L, S77A, D79V, K80*, K80E, K80H, K80L, K80N, K80Q, K80V, K80W, D81A, D81G, D81L, D81R, D81S, D81T, D81V, D81W, I82V, L90F, E99Q, E99R, L100S, K103A, K103R, K104N, K104W, D105N, V106A, V106D, V106E, V106H, V106K, V106L, V106M, V106N, V106Q, V106W, V106Y, K107A, K107C, K107L, K107M, K107T, K107V, K107W, N110M, N110R, N110V, D111A, D111E, D111M, D111N, D111Q, D111R, D111V, D111W, V113T, T114C, T114S, Y116D, Y116N, Y116R, 5117*, S117D, S117H, S117N, S117P, E118*, E118A, E118D, E118G, E118L, E119G, E119W, T120I, T120L, T120M, T120V, T120W, D122*, D122H, D122R, Y123W, Y124C, Y124I, Y124K, Y124L, Y124M, Y124Q, Y124R, Y124T, Y124V, Y124W, D125C, D125G, D125K, D125Q, D125R, N126V, R127D, R127H, R127K, R127L, R127M, R127Q, R127W, V128C, V128L, V128T, D131V, Q135*, Q135A, Q135D, Q135E, Q135K, Q135M, Q135Y, D138K, D138L, D138M, D138Q, D138R, D138S, D138V, D138W, E139W, D142R, D142W, Y145*, Y145H, Y145L, Y145N, Y145V, P147A, P147C, P147D, P147F, P147G, P147L, P147M, P147R, P147S, P147T, P147V, K148A, K148D, K148L, K148V, F149L, F149M, F149N, E150D, E150H, E150K, E150L, E150M, E150N, E150R, E150V, E150W, E150Y, G151A, G151C, G151D, G151L, G151N, G151P, G151S, G151W, K152D, K152L, K152R, G164D, G164E, G164H, G164S, G164V, V167D, V167E, V167L, V167P, V167Q, V167R, V167W, H168N, L170A, L170D, L170E, L170F, L170H, L170K, L170M, L170N, L170P, L170Q, L170R, L170S, L170V, L170W, L170Y, D171A, D171C, D171E, D171K, D171L, D171M, D171Q, D171R, D171V, D171W, D171Y, I173C, D174H, D174M, D174N, D174V, D174W, F175Y, N177M, Q178*, Q178A, Q178K, Q178R, Q178W, I179T, S181C, S181D, S181F, S181G, S181N, S181P, S181Q, S181T, S181V, S181W, E185M, E185R, E185V, E185W, S186D, S186E, S186H, S186I, S186K, S186L, S186M, S186N, S186Q, S186R, S186V, S186W, K187C, K187D, K187G, K187R, K187S, K187V, K187W, Y188P, E189L, E189V, E189W, S199C, S199L, S199M, S199Y, E200D, E200F, E200K, E200L, E200M, E200N, E200R, E200W, A203C, A203D, A203E, A203G, A203L, A203M, A203P, A203R, A203S, A203T, A203V, A203W, N204L, N204M, N204V, N204W, N204Y, L205I, D207A, D207C, D207E, D207G, D207N, D207Q, D207S, D207V, D207W, S208A, S208C, S208D, S208G, S208L, S208Q, S208T, S208V, S208W, S210T, Q215R, Q215M, Q215L, Q215*, S217V, T218A, T218L, T218Q, T218R, T218V, G222D, E224A, E224P, N227A, N227Q, N227R, N227S, N227T, N227K, D230*, D230R, D230T, D230W, E232D, E232V, N233H, N233Q, N233R, N233W, W234R, G235W, G235A, G235E, G235F, G235H, G235I, G235L, G235M, G235N, G235P, G235S, G235V, S237C, S237G, S237M, S237N, S237W, S237Y, Y244C, Y244E, Y244M, L249H, L249K, L249Q, L249R, L249W, L249Y, S251L, S251N, S251R, S251W, N252P, N252C, G253D, G253W, F254I, F254L, F254M, F254Y, Q256E, Q256R, N260*, N260A, N260C, N260E, N260I, N260K, N260L, N260M, N260Q, N260R, N260V, N260W, N260Y, E261*, E261A, E261D, E261R, E261W, Q262*, Q262F, Q262H, Q262W, Q262Y, M263K, M263L, M263Q, D264*, D264C, D264E, Y265F, N267S, N267T, W268C, W268E, W268M, W268R, Y270F, A271D, A271G, H272D, H272I, Y281P, Y282E, Y282N, Y281R, H272W, N273W, K274R, K274A, K274H, F276A, F276C, F276K, F276N, F276G, F276L, F276M, F276P, F276S, F276V, F276W, I278A, I278K, I278N, I278Q, I278V, S279C, S279D, S279E, S279G, S279N, D280C, D280E, Y281*, Y281A, Y281C, Y281H, Y281K, Y281N, Y281P, Y282E, Y282N, H283I, A284I, A284L, A284N, A284P, A284T, A284V, T287N, S288D, S288K, S288N, V290I, K291L, K291R, K291V, T296C, E300A, E300D, H301C, H301N, H301R, T303A, T303C, T303G, T303K, T303Q, T303R, T303W, D304C, D304M, L305M, L305N, S306C, K308A, K308D, K308G, K308I, K308L, K308Q, K308S, K308T, K308V, K308Y, K309A, K309C, K309D, K309H, K309L, K309M, K309N, K309Q, K309S, K309T, K309I, K312A, K312L, K312M, K312N, K312Q, K312S, K312W, E314I, E314L, E314V, L315I, L315V, R319A, N323R and combinations thereof, preferably in an amount of active protein of 0.00001 to 5 wt. %, preferably 0.0003 to 0.5 wt. %, more preferably 0.0005 to 0.25 wt. %, particularly preferably 0.001 to 0.25 wt. %, based on the total weight of the agent;
(B) at least one fragrance, preferably in an amount of 0.01 to 5 wt. %, preferably 0.05 to 3 wt. %, particularly preferably 0.1 to 1 wt. %, based on the total weight of the agent, wherein the fragrance is selected from the group consisting of α-isomethyl ionone, benzyl salicylate, citronellol, coumarin, hexyl cinnamal, linalool, pentanoic acid, 2-methylethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-iron, β-phenylethyl benzoate, α-santalol, cedrol, cedryl acetate, cedryl formate, cyclohexyl salicyate, γ-dodecalactone, β-phenylethylphenylacetate, 2-methyl-6-methylene-7-octen-2-ol (dihydroderivatives), 3A,4,5,6,7,7A-hexahydro-4,7-methano-1H-inden-5(or 6)-ylacetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-ylacetate, 3,7-dimethyl-3-octanol, 4-(2,6,6-trimethylcyclohex-1-en-1-yl)but-3-en-2-one, hedione, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allyl heptanoate, nerol, pin-2(3)-ene, diphenyl ether, methyl 2-naphthyl ether, methyl benzoate, 5-heptyldihydro-2(3H)-furanone, isopropyl ester myristic acid, γ-decalactone, ethyl 2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethylphenyl ethyl acetal, b-4-dimethyl-3-cyclohexene-1-propanal, peppermint oil, d-p-mentha-1(6),8-dien-2-one and mixtures thereof, and
(C) (at least one ingredient, preferably in an amount of 0.01 to 99.9 wt. %, based on the total weight of the agent.

In preferred embodiments, the at least one dispersin and the at least one fragrance interact synergistically and lead to an increased fragrance intensity of the fragrance contained in the washing and/or cleaning agent on a textile or surface treated with the agent when the fragrance intensity is determined as described in Example 1.

In preferred embodiments, the at least one dispersin and the at least one fragrance interact synergistically and lead to a reduced malodor of a textile or surface treated with the washing and/or cleaning agent when the malodor is determined as described in Example 1.

The washing and/or cleaning agent according to the invention is preferably a textile washing agent, in particular a liquid textile washing agent, very particularly preferably a pre-portioned textile washing agent. Preferably, the agent is flowable under standard conditions (20° C., 1013 mbar). Further preferably, the washing and/or cleaning agent is substantially free of boron-containing compounds, preferably free of boron-containing compounds; and/or has a pH in a range of approximately 6 to approximately 11, in particular of approximately 6.5 to approximately 10.5, more preferably of approximately 7 to approximately 10, and particularly preferably of approximately 8 to approximately 9, in a 1 wt. % solution in deionized water at 20° C.

Further objects of the invention relate to
a method for cleaning textiles and/or hard surfaces, in particular dishes, using a washing and/or cleaning agent according to the invention, wherein the method is preferably carried out in a temperature range from approximately 20° C. to approximately 60° C., preferably approximately 40° C., particularly preferably approximately 30° C.,
the use of a washing and/or cleaning agent according to the invention for cleaning textiles and/or hard surfaces, in particular dishes, in particular for deep cleaning, preferably in a temperature range of approximately 20° C. to approximately 60° C., preferably approximately 40° C., particularly preferably approximately 30° C.,
the use of a dispersin in a washing and/or cleaning agent according to the invention for reducing the malodor of a textile or a surface which has been treated with the washing and/or cleaning agent, preferably in a temperature range of approximately 20° C. to approximately 60° C., preferably approximately 40° C., particularly preferably approximately 30° C., and
the use of a dispersin in a washing and/or cleaning agent according to the invention for increasing the fragrance intensity of the fragrance contained in the agent on a textile or a surface which has been treated with the agent, preferably in a temperature range of approximately 20° C. to approximately 60° C., preferably 40° C., particularly preferably approximately 30° C.

These and other aspects, features and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate but not to limit the invention and that, in particular, the invention is not limited to these examples.

Unless indicated otherwise, all percentages are indicated in terms of weight percent (wt. %).

Numerical ranges that are indicated in the format “from x to y” also include the stated values. If several preferred numerical ranges are specified in this format, it is readily understood that any ranges resulting from the combination of the various endpoints are also included.

“At least one,” as used herein, means one or more, i.e., 1, 2, 3, 4, 5, 6, 7, 8, 9, 10, 11, 12, 13, 14 or more.

The term “washing agent and/or cleaning agent,” “washing agent and cleaning agent” or “washing agent or cleaning agent” as used herein is synonymous with the term “agent” and denotes a composition for cleaning textiles and/or hard surfaces, in particular dishes, as explained in the description.

“Approximately,” “about,” or “roughly,” as used herein in reference to a numerical value, refers to the corresponding numerical value ±10%, preferably ±5%.

“Liquid,” as used herein, includes liquids and gels as well as pasty compositions. It is preferred that the liquid compositions be flowable and pourable at room temperature, but it is also possible for them to have a limit of liquidity.

A substance, e.g., a composition or an agent, is solid in accordance with the definition of the invention if it is in a solid state of aggregation at 25° C. and 1013 mbar.

A substance, e.g. a composition or an agent, is liquid in accordance with the definition of the invention if it is in a liquid state of aggregation at 25° C. and 1013 mbar. Liquid also includes gel form.

“Variant,” as used herein, refers to naturally or artificially generated variations of a native enzyme which have an amino acid sequence which is modified from the reference form.

The term “deep cleaning” as used herein means the dissolution or removal of constituents of organic substances, e.g., sebum, cell residues, body soil, bio-buildups, biofilms (including e.g., polysaccharides, PNAG (poly-N-acetyl-glucosamine)), body oils or other components contained in organic substances, not only at the level visible to the naked eye, but also the dissolution or removal of microbially induced deposits not visible to the naked eye, in particular deposits, bio-buildups and the like between individual fibers of textiles.

The term “textile” as used herein means any textile material, including yarns, yarn precursors, fibers, nonwovens, natural materials, synthetic materials and all other textile materials, fabrics made from these materials, and products made from fabrics (e.g. garments and other articles). The textile or fabric can be in the form of knits, wovens, denims, nonwovens, felts, yarns and terry cloth. The textile can be based on cellulose, for example natural cellulose fibers such as cotton, flax/linen, jute, ramie, sisal or coconut fibers, or synthetically produced cellulose fibers (e.g. from pulp) such as viscose/rayon, cellulose acetate fibers (tricell), lyocell or mixtures thereof. The textile or fabric can also consist of non-cellulosic fibers, e.g. natural polyamides such as wool, camel, cashmere, mohair, rabbit hair and silk, or synthetic polymers such as nylon, aramid, polyester, acrylic, polypropylene and spandex/elastane or mixtures thereof, as well as mixtures of cellulosic fibers and non-cellulosic fibers. Examples of mixtures are mixtures of cotton and/or rayon/viscose with one or more accompanying materials such as wool, synthetic fibers (e.g. polyamide fibers, acrylic fibers, polyester fibers, polyvinyl chloride fibers, polyurethane fibers, polyurea fibers, aramid fibers) and/or cellulosic fibers (e.g. rayon/viscose, ramie, flax/linen, jute, cellulose acetate fibers, lyocell). The fabric can be conventional washable laundry, e.g. soiled household laundry. When the term “fabric” or “garment” is used, it is intended to include the broader term “textiles.”

The present invention is based on the surprising finding of the inventors that the dispersin according to the invention not only combats the cause of malodor, in particular malodor caused by organic coatings and/or body soil, but also interacts synergistically with the at least one fragrance contained in the washing and/or cleaning agent. In preferred embodiments, the synergistic interaction of dispersin and fragrance not only leads to a reduction in malodor, but also to a freshness boost and increased fragrance intensity. In further preferred embodiments, this effect, in particular the increased fragrance intensity, also occurs with a reduced amount of fragrance (compared to a conventional washing and cleaning agent), wherein this effect is present both after washing and during wear, e.g., after the first sweating, and on the dry laundry.

The washing and/or cleaning agent according to the invention comprises
(A) at least one dispersin, wherein the dispersin exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68,71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion, and
(B) at least one fragrance.

This is particularly surprising insofar as no such combination has previously been associated with an improved malodor reduction in washing agents and/or cleaning agents.

Dispersins belong to the group of hexosaminidases or β-N-acetylglucosamininidases (EC 3.2.1.52). These are polypeptides that catalyze the hydrolysis of N-acetyl-D-hexosamine or N-acetylglucosamine polymers. Dispersin B is a β-hexosaminidase that specifically hydrolyzes β-1,6-glycosidic bonds of acetylglucosamine polymers found e.g., in biofilm, EPSs (extracellular polymer structures), cell debris, bio-buildups and other so-called biosoils (i.e., stains of biological origin).

According to the invention, a dispersin is used which exhibits β-1,6-N-acetylglucosaminidase activity and which comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion.

In preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion selected from the group consisting of D2A, D2L, D2N, D2R, D2V, D2W, Q3F, Q3I, Q3L, Q3M, Q3P, Q3V, Q3Y, Q3T, S12A, H15F, H15Y, T17W, T17C, T17E, T17F, T17M, T17R, T17V, V18L, E19D, E19N, E19P, K22A, K22M, K22V, S23C, S23E, S23I, S23L, S23R, S23T, S23V, V25R, D26M, Y30*, Y30D, Y30L, Y30M, Y30N, Y30R, Y30T, Y30V, G32L, G32M, G32R, N43*, N43H, N43L, E44*, N45D, N45L, N45V, A49W, A49Y, Y52*, Y52M, G54L, G54M, G54N, S56T, S56W, S57W, E58N, N59A, N59C, N59D, N59E, N59F, N59M, N59R, N59V, N59W, T60V, N62C, N62D, N62H, N62Q, N62W, T63C, T63D, T63L, T63N, T63R, T63V, K67A, K67L, N68L, N68Q, L71H, L71N, L71R, L71V, L71W, S72*, S72C, S72D, S72E, S72F, S72G, S72I, S72M, S72N, S72R, S72T, S72Y, I74L, S77A, D79V, K80*, K80E, K80H, K80L, K80N, K80Q, K80V, K80W, D81A, D81G, D81L, D81R, D81S, D81T, D81V, D81W, I82V, L90F, E99Q, E99R, L100S, K103A, K103R, K104N, K104W, D105N, V106A, V106D, V106E, V106H, V106K, V106L, V106M, V106N, V106Q, V106W, V106Y, K107A, K107C, K107L, K107M, K107T, K107V, K107W, N110M, N110R, N110V, D111A, D111E, D111M, D111N, D111Q, D111R, D111V, D111W, V113T, T114C, T114S, Y116D, Y116N, Y116R, 5117*, S117D, S117H, S117N, S117P, E118*, E118A, E118D, E118G, E118L, E119G, E119W, T120I, T120L, T120M, T120V, T120W, D122*, D122H, D122R, Y123W, Y124C, Y124I, Y124K, Y124L, Y124M, Y124Q, Y124R, Y124T, Y124V, Y124W, D125C, D125G, D125K, D125Q, D125R, N126V, R127D, R127H, R127K, R127L, R127M, R127Q, R127W, V128C, V128L, V128T, D131V, Q135*, Q135A, Q135D, Q135E, Q135K, Q135M, Q135Y, D138K, D138L, D138M, D138Q, D138R, D138S, D138V, D138W, E139W, D142R, D142W, Y145*, Y145H, Y145L, Y145N, Y145V, P147A, P147C, P147D, P147F, P147G, P147L, P147M, P147R, P147S, P147T, P147V, K148A, K148D, K148L, K148V, F149L, F149M, F149N, E150D, E150H, E150K, E150L, E150M, E150N, E150R, E150V, E150W, E150Y, G151A, G151C, G151D, G151L, G151N, G151P, G151S, G151W, K152D, K152L, K152R, G164D, G164E, G164H, G164S, G164V, V167D, V167E, V167L, V167P, V167Q, V167R, V167W, H168N, L170A, L170D, L170E, L170F, L170H, L170K, L170M, L170N, L170P, L170Q, L170R, L170S, L170V, L170W, L170Y, D171A, D171C, D171E, D171K, D171L, D171M, D171Q, D171R, D171V, D171W, D171Y, I173C, D174H, D174M, D174N, D174V, D174W, F175Y, N177M, Q178*, Q178A, Q178K, Q178R, Q178W, I179T, S181C, S181D, S181F, S181G, S181N, S181P, S181Q, S181T, S181V, S181W, E185M, E185R, E185V, E185W, S186D, S186E, S186H, S186I, S186K, S186L, S186M, S186N, S186Q, S186R, S186V, S186W, K187C, K187D, K187G, K187R, K187S, K187V, K187W, Y188P, E189L, E189V, E189W, S199C, S199L, S199M, S199Y, E200D, E200F, E200K, E200L, E200M, E200N, E200R, E200W, A203C, A203D, A203E, A203G, A203L, A203M, A203P, A203R, A203S, A203T, A203V, A203W, N204L, N204M, N204V, N204W, N204Y, L205I, D207A, D207C, D207E, D207G, D207N, D207Q, D207S, D207V, D207W, S208A, S208C, S208D, S208G, S208L, S208Q, S208T, S208V, S208W, S210T, Q215R, Q215M, Q215L, Q215*, S217V, T218A, T218L, T218Q, T218R, T218V, G222D, E224A, E224P, N227A, N227Q, N227R, N227S, N227T, N227K, D230*, D230R, D230T, D230W, E232D, E232V, N233H, N233Q, N233R, N233W, W234R, G235W, G235A, G235E, G235F, G235H, G235I, G235L, G235M, G235N, G235P, G235S, G235V, S237C, S237G, S237M, S237N, S237W, S237Y, Y244C, Y244E, Y244M, L249H, L249K, L249Q, L249R, L249W, L249Y, S251L, S251N, S251R, S251W, N252P, N252C, G253D, G253W, F254I, F254L, F254M, F254Y, Q256E, Q256R, N260*, N260A, N260C, N260E, N260I, N260K, N260L, N260M, N260Q, N260R, N260V, N260W, N260Y, E261*, E261A, E261D, E261R, E261W, Q262*, Q262F, Q262H, Q262W, Q262Y, M263K, M263L, M263Q, D264*, D264C, D264E, Y265F, N267S, N267T, W268C, W268E, W268M, W268R, Y270F, A271D, A271G, H272D, H272I, Y281P, Y282E, Y282N, Y281R, H272W, N273W, K274R, K274A, K274H, F276A, F276C, F276K, F276N, F276G, F276L, F276M, F276P, F276S, F276V, F276W, I278A, I278K, I278N, I278Q, I278V, S279C, S279D, S279E, S279G, S279N, D280C, D280E, Y281*, Y281A, Y281C, Y281H, Y281K, Y281N, Y281P, Y282E, Y282N, H283I, A284I, A284L, A284N, A284P, A284T, A284V, T287N, S288D, S288K, S288N, V290I, K291L, K291R, K291V, T296C, E300A, E300D, H301C, H301N, H301R, T303A, T303C, T303G, T303K, T303Q, T303R, T303W, D304C, D304M, L305M, L305N, S306C, K308A, K308D, K308G, K308I, K308L, K308Q, K308S, K308T, K308V, K308Y, K309A, K309C, K309D, K309H, K309L, K309M, K309N, K309Q, K309S, K309T, K309I, K312A, K312L, K312M, K312N, K312Q, K312S, K312W, E314I, E314L, E314V, L315I, L315V, R319A, N323R and combinations thereof.

In further preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 22, 23, 25, 30, 49, 56, 59, 62, 63, 68, 72, 74, 77, 82, 90, 99, 100, 114, 123, 124, 125, 135, 138, 170, 171, 173, 174, 175, 178, 179, 181, 185, 186, 187, 188, 189, 199, 203, 204, 205, 207, 210, 215, 218, 227, 232, 235, 237, 244, 252, 256, 260, 262, 263, 264, 265, 267, 270, 272, 273, 274, 276, 278, 279, 280, 281, 282, 283, 284, 288, 290, 291, 296, 303, 304, 305, 306, 308, 309, 312, 314, 315, 319 and 321, has at least one amino acid substitution and/or deletion which is selected from the group consisting of D2V, Q3F, Q3I, S12A, H15Y, T17W, T17E, V18L, K22M, S23I, S23C, S23T, S23L, S23V, S23E, V25R, Y30L, A49W, A49Y, S56T, N59D, N59C, N59E, N59R, N59F, N59W, N59V, N62C, N62D, T63C, N68Q, S72D, S72E, I74L, S77A, I82V, L90F, E99Q, L100S, T114S, T114C, Y123W, Y124I, Y124M, Y124R, Y124V, Y124Q, Y124T, Y124K, D125R, D125C, D125G, D125K, D125Q, Q135M, D138R, D138K, D138Q, L170D, L170K, L170S, L170H, D171E, D171K, D171Y, D171M, D171Q, D171L, I173C, D174W, D174H, D174M, D174N, F175Y, Q178K, I179T, S181T, S181F, S181Q, S181G, S181N, S181C, E185R, E185M, E185V, S186K, S186M, S186R, S186H, K187G, Y188P, E189V, S199C, S199L, A203G, A203E, A203V, N204L, N204Y, N204V, L205I, D207N, D207S, D207C, D207G, S210T, Q215R, T218Q, N227T, N227K, E232D, G235W, S237W, Y244M, Y244C, N252P, N252C, Q256E, N260Q, Q262H, M263Q, D264E, Y265F, N267T, N267S, Y270F, H272M, H272P, H272I, H272V, N273W, K274H, F276A, F276N, F276K, F276C, I278V, S279N, S279D, S279G, D280E, D280C, Y281P, Y282N, H283I, A284T, A284L, A284I, S288K, V290I, K291R, T296C, T303Q, D304M, D304C, L305M, L305N, S306C, K308D, K308A, K308V, K308Q, K308S, K308Y, K308G, K308L, K308T, K308I, K309C, K309L, K309D, K309Q, K309N, K309T, K309A, K309S, K309M, K309H, K312A, K312Q, K312S, K312W, K312L, K312N, E314L, E314V, E314I, L315I, R319A, N323R and combinations thereof.

In further preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 3, 15, 17, 49, 59, 140, 163, 186, 207, 215, 218, 225, 227, 232, 235, 237, 252, 260, 267, 272, 279, 281, 288, 308, 309 and 312, has at least one amino acid substitution and/or deletion which is selected from the group consisting of Q3F, Q3I, H15Y, T17W, A49W, N59E, V140I, S163P, S186R, D207N, Q215K, T218Q, S225G, N227T, E232D, G235W, S237W, N252P, N260Q, N267T, H272V, H272P, S279D, Y281P, S288P, K308Q, K309E, K312Q and combinations thereof.

In particularly preferred embodiments, a dispersin is used which exhibits β-1,6-N-acetylglucosaminidase activity and which comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and has, based on the numbering according to SEQ ID NO:1, at at least one of the positions corresponding to positions 3, 15, 49, 59, 163, 186, 225, 227, 232, 235, 252, 260, 272, 279, 281, 308, 309 and 312, at least one amino acid substitution and/or deletion which is selected from the group consisting of Q3I, H15Y, A49W, N59E, S163P, S186R, S225G, N227T, E232D, G235W, N252P, N260Q, H272V, S279D, Y281P, K308Q, K309E, K312Q and combinations thereof.

In preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and has at least one and increasingly preferably two, three, four, five, six, seven, eight or nine amino acid substitutions, wherein the dispersin has an amino acid substitution combination selected from the group consisting of Q3I-A49W, Q3I-N59E, Q3I-S163P, Q3I-S186R, Q3I-Q215K, Q3I-S225G, Q3I-N227T, Q3I-N252P, Q3I-N267T, Q3I-F276A, Q3I-Y281P, Q3I-K308Q, Q3I-K308E, Q3I-K309E, Q3I-K312Q, Q3I-K312E, Q3I-H15Y, Q3I-E232D, Q3I-G235W, Q3I-N260Q, Q3I-H272V, Q3I-S279D, Q3I-H15Y-A49W, Q3I-H15Y-N59E, Q3I-H15Y-S163P, Q3I-H15Y-S186R, Q3I-H15Y-S225G, Q3I-H15Y-N227T, Q3I-H15Y-E232D, Q3I-H15Y-G235W, Q3I-H15Y-N252P, Q3I-H15Y-N260Q, Q3I-H15Y-H272V, Q3I-H15Y-S279D, Q3I-H15Y-Y281P, Q3I-H15Y-K308Q, Q3I-H15Y-K309E, Q3I-H15Y-K312Q, Q3I-A49W-N59E, Q3I-A49W-S163P, Q3I-A49W-S186R, Q3I-A49W-S225G, Q3I-A49W-N227T, Q3I-A49W-E232D, Q3I-A49W-G235W, Q3I-A49W-N252P, Q3I-A49W-N260Q, Q3I-A49W-H272V, Q3I-A49W-S279D, Q3I-A49W-Y281P, Q3I-A49W-K308Q, Q3I-A49W-K309E, Q3I-A49W-K312Q, Q3I-N59E-S163P, Q3I-N59E-S186R, Q3I-N59E-S225G, Q3I-N59E-N227T, Q3I-N59E-E232D, Q3I-N59E-G235W, Q3I-N59E-N252P, Q3I-N59E-N260Q, Q3I-N59E-H272V, Q3I-N59E-S279D, Q3I-N59E-Y281P, Q3I-N59E-K308Q, Q3I-N59E-K309E, Q3I-N59E-K312Q, Q3I-S163P-S186R, Q3I-S163P-S225G, Q3I-S163P-N227T, Q3I-S163P-E232D, Q3I-S163P-G235W, Q3I-S163P-N252P, Q3I-S163P-N260Q, Q3I-S163P-H272V, Q3I-S163P-S279D, Q3I-S163P-Y281P, Q3I-S163P-K308Q, Q3I-S163P-K309E, Q3I-S163P-K312Q, Q3I-S186R-S225G, Q3I-S186R-N227T, Q3I-S186R-E232D, Q3I-S186R-G235W, Q3I-S186R-N252P, Q3I-S186R-N260Q, Q3I-S186R-H272V, Q3I-S186R-S279D, Q3I-S186R-Y281P, Q3I-S186R-K308Q, Q3I-S186R-K309E, Q3I-S186R-K312Q, Q3I-S225G-N227T, Q3I-S225G-E232D, Q3I-S225G-G235W, Q3I-S225G-N252P, Q3I-S225G-N260Q, Q3I-S225G-H272V, Q3I-S225G-S279D, Q3I-S225G-Y281P, Q3I-S225G-K308Q, Q3I-S225G-K309E, Q3I-S225G-K312Q, Q3I-N227T-E232D, Q3I-N227T-G235W, Q3I-N227T-N252P, Q3I-N227T-N260Q, Q3I-N227T-H272V, Q3I-N227T-S279D, Q3I-N227T-Y281P, Q3I-N227T-K308Q, Q3I-N227T-K309E, Q3I-N227T-K312Q, Q3I-E232D-G235W, Q3I-E232D-N252P, Q3I-E232D-N260Q, Q3I-E232D-H272V, Q3I-E232D-S279D, Q3I-E232D-Y281P, Q3I-E232D-K308Q, Q3I-E232D-K309E, Q3I-E232D-K312Q, Q3I-G235W-N252P, Q3I-G235W-N260Q, Q3I-G235W-H272V, Q3I-G235W-S279D, Q3I-G235W-Y281P, Q3I-G235W-K308Q, Q3I-G235W-K309E, Q3I-G235W-K312Q, Q3I-N252P-N260Q, Q3I-N252P-H272V, Q3I-N252P-S279D, Q3I-N252P-Y281P, Q3I-N252P-K308Q, Q3I-N252P-K309E, Q3I-N252P-K312Q, Q3I-N260Q-H272V, Q3I-N260Q-S279D, Q3I-N260Q-Y281P, Q3I-N260Q-K308Q, Q3I-N260Q-K309E, Q3I-N260Q-K312Q, Q3I-H272V-S279D, Q3I-H272V-Y281P, Q3I-H272V-K308Q, Q3I-H272V-K309E, Q3I-H272V-K312Q, Q3I-S279D-Y281P, Q3I-S279D-K308Q, Q3I-S279D-K309E, Q3I-S279D-K312Q, Q3I-Y281P-K308Q, Q3I-Y281P-K309E, Q3I-Y281P-K312Q, Q3I-K308Q-K309E, Q3I-K308Q-K312Q, Q3I-K309E-K312Q, Q3I-Q225G-N227T-N252P-N260Q-S186R-K308Q-K309E-K312Q, Q3I-S163P-Q225G-N227T-N252P-N260Q-K308Q-K309E-K312Q, Q3F-A49W, Q3F-N59E, Q3F-S163P, Q3F-S186R, Q3F-Q215K, Q3F-S225G, Q3F-N227T, Q3F-N252P, Q3F-N267T, Q3F-F276A, Q3F-Y281P, Q3F-K308Q, Q3F-K308E, Q3F-K309E, Q3F-K312E, Q3F-K312Q, Q3F-A49W-Q215K-F276A-K308E-K309E-K312E-N227T, Q3F-A49W-Q215K-F276A-K308E-K309E-K312E-N267T, Q3F-A49W-Q215K-F276A-K308E-K309E-N227T-N267T, Q3F-A49W-Q215K-F276A-K308E-K312E-N227T-N267T, Q3F-A49W-Q215K-F276A-K309E-K312E-N227T-N267T, Q3F-A49W-Q215K-K308E-K309E-K312E-N227T-N267T, Q3F-A49W-F276A-K308E-K309E-K312E-N227T-N267T, Q3F-Q215K-F276A-K308E-K309E-K312E-N227T-N267T, Q3F-Q215K-N227T-N252P-N267T-F276A-K308E-K309E-K312E, Q3F-S163P-Q215K-N227T-N252P-N267T-K308E-K309E-K312E, Q3F-A49W-Q215K-F276A-K308E-K309E-K312E-N227T-N267T, H15Y-A49W, H15Y-N59E, H15Y-S163P, H15Y-S186R, H15Y-S225G, H15Y-N227T, H15Y-E232D, H15Y-235W, H15Y-N252P, H15Y-N260Q, H15Y-H272V, H15Y-S279D, H15Y-Y281P, H15Y-K308Q, H15Y-K309E, H15Y-K312Q, H15Y-A49W-N59E, H15Y-A49W-S163P, H15Y-A49W-S186R, H15Y-A49W-S225G, H15Y-A49W-N227T, H15Y-A49W-E232D, H15Y-A49W-G235W, H15Y-A49W-N252P, H15Y-A49W-N260Q, H15Y-A49W-H272V, H15Y-A49W-S279D, H15Y-A49W-Y281P, H15Y-A49W-K308Q, H15Y-A49W-K309E, H15Y-A49W-K312Q, H15Y-N59E-S163P, H15Y-N59E-S186R, H15Y-N59E-S225G, H15Y-N59E-N227T, H15Y-N59E-E232D, H15Y-N59E-G235W, H15Y-N59E-N252P, H15Y-N59E-N260Q, H15Y-N59E-H272V, H15Y-N59E-S279D, H15Y-N59E-Y281P, H15Y-N59E-K308Q, H15Y-N59E-K309E, H15Y-N59E-K312Q, H15Y-S163P-S186R, H15Y-S163P-S225G, H15Y-S163P-N227T, H15Y-S163P-E232D, H15Y-S163P-G235W, H15Y-S163P-N252P, H15Y-S163P-N260Q, H15Y-S163P-H272V, H15Y-S163P-S279D, H15Y-S163P-Y281P, H15Y-S163P-K308Q, H15Y-S163P-K309E, H15Y-S163P-K312Q, H15Y-S186R-S225G, H15Y-S186R-N227T, H15Y-S186R-E232D, H15Y-S186R-G235W, H15Y-S186R-N252P, H15Y-S186R-N260Q, H15Y-S186R-H272V, H15Y-S186R-S279D, H15Y-S186R-Y281P, H15Y-S186R-K308Q, H15Y-S186R-K309E, H15Y-S186R-K312Q, H15Y-S225G-N227T, H15Y-S225G-E232D, H15Y-S225G-G235W, H15Y-S225G-N252P, H15Y-S225G-N260Q, H15Y-S225G-H272V, H15Y-S225G-S279D, H15Y-S225G-Y281P, H15Y-S225G-K308Q, H15Y-S225G-K309E, H15Y-S225G-K312Q, H15Y-N227T-E232D, H15Y-N227T-G235W, H15Y-N227T-N252P, H15Y-N227T-N260Q, H15Y-N227T-H272V, H15Y-N227T-S279D, H15Y-N227T-Y281P, H15Y-N227T-K308Q, H15Y-N227T-K309E, H15Y-N227T-K312Q, H15Y-E232D-G235W, H15Y-E232D-N252P, H15Y-E232D-N260Q, H15Y-E232D-H272V, H15Y-E232D-S279D, H15Y-E232D-Y281P, H15Y-E232D-K308Q, H15Y-E232D-K309E, H15Y-E232D-K312Q, H15Y-G235W-N252P, H15Y-G235W-N260Q, H15Y-G235W-H272V, H15Y-G235W-S279D, H15Y-G235W-Y281P, H15Y-G235W-K308Q, H15Y-G235W-K309E, H15Y-G235W-K312Q, H15Y-N252P-N260Q, H15Y-N252P-H272V, H15Y-N252P-S279D, H15Y-N252P-Y281P, H15Y-N252P-K308Q, H15Y-N252P-K309E, H15Y-N252P-K312Q, H15Y-N260Q-H272V, H15Y-N260Q-S279D, H15Y-N260Q-Y281P, H15Y-N260Q-K308Q, H15Y-N260Q-K309E, H15Y-N260Q-K312Q, H15Y-H272V-S279D, H15Y-H272V-Y281P, H15Y-H272V-K308Q, H15Y-H272V-K309E, H15Y-H272V-K312Q, H15Y-S279D-Y281P, H15Y-S279D-K308Q, H15Y-S279D-K309E, H15Y-S279D-K312Q, H15Y-Y281P-K308Q, H15Y-Y281P-K309E, H15Y-Y281P-K312Q, H15Y-K308Q-K309E, H15Y-K308Q-K312Q, H15Y-K309E-K312Q, A49W-N59E, A49W-S163P, A49W-S186R, A49W-Q215K, A49W-S225G, A49W-N227T, A49W-N252P, A49W-N267T, A49W-F276A, A49W-Y281P, A49W-K308Q, A49W-K308E, A49W-K309E, A49W-K312Q, A49W-K312E, A49W-E232D, A49W-G235W, A49W-N260Q, A49W-H272V, A49W-S279D, A49W-N59E-S163P, A49W-N59E-S186R, A49W-N59E-S225G, A49W-N59E-N227T, A49W-N59E-E232D, A49W-N59E-G235W, A49W-N59E-N252P, A49W-N59E-N260Q, A49W-N59E-H272V, A49W-N59E-S279D, A49W-N59E-Y281P, A49W-N59E-K308Q, A49W-N59E-K309E, A49W-N59E-K312Q, A49W-S163P-S186R, A49W-S163P-S225G, A49W-S163P-N227T, A49W-S163P-E232D, A49W-S163P-G235W, A49W-S163P-N252P, A49W-S163P-N260Q, A49W-S163P-H272V, A49W-S163P-S279D, A49W-S163P-Y281P, A49W-S163P-K308Q, A49W-S163P-K309E, A49W-S163P-K312Q, A49W-S186R-S225G, A49W-S186R-N227T, A49W-S186R-E232D, A49W-S186R-G235W, A49W-S186R-N252P, A49W-S186R-N260Q, A49W-S186R-H272V, A49W-S186R-S279D, A49W-S186R-Y281P, A49W-S186R-K308Q, A49W-S186R-K309E, A49W-S186R-K312Q, A49W-S225G-N227T, A49W-S225G-E232D, A49W-S225G-G235W, A49W-S225G-N252P, A49W-S225G-N260Q, A49W-S225G-H272V, A49W-S225G-S279D, A49W-S225G-Y281P, A49W-S225G-K308Q, A49W-S225G-K309E, A49W-S225G-K312Q, A49W-N227T-E232D, A49W-N227T-G235W, A49W-N227T-N252P, A49W-N227T-N260Q, A49W-N227T-H272V, A49W-N227T-S279D, A49W-N227T-Y281P, A49W-N227T-K308Q, A49W-N227T-K309E, A49W-N227T-K312Q, A49W-E232D-G235W, A49W-E232D-N252P, A49W-E232D-N260Q, A49W-E232D-H272V, A49W-E232D-S279D, A49W-E232D-Y281P, A49W-E232D-K308Q, A49W-E232D-K309E, A49W-E232D-K312Q, A49W-G235W-N252P, A49W-G235W-N260Q, A49W-G235W-H272V, A49W-G235W-S279D, A49W-G235W-Y281P, A49W-G235W-K308Q, A49W-G235W-K309E, A49W-G235W-K312Q, A49W-N252P-N260Q, A49W-N252P-H272V, A49W-N252P-S279D, A49W-N252P-Y281P, A49W-N252P-K308Q, A49W-N252P-K309E, A49W-N252P-K312Q, A49W-N260Q-H272V, A49W-N260Q-S279D, A49W-N260Q-Y281P, A49W-N260Q-K308Q, A49W-N260Q-K309E, A49W-N260Q-K312Q, A49W-H272V-S279D, A49W-H272V-Y281P, A49W-H272V-K308Q, A49W-H272V-K309E, A49W-H272V-K312Q, A49W-S279D-Y281P, A49W-S279D-K308Q, A49W-S279D-K309E, A49W-S279D-K312Q, A49W-Y281P-K308Q, A49W-Y281P-K309E, A49W-Y281P-K312Q, A49W-K308Q-K309E, A49W-K308Q-K312Q, A49W-K309E-K312Q, A49W-N227T-F276A-K308E, A49W-S186R-N227T-F276A, A49W-S186R-N227T-K308E, A49W-N227T-A269C-Y282C-K308E, A49W-S186R-N227T-F276A-K308E, A49W-Y124R-N227T-F276A-K308E, A49W-Y124R-S186R-N227T-F276A, A49W-Y124R-S186R-N227T-K308E, A49W-A269C-F276A-Y282C-K308E, A49W-N227T-A269C-F276A-Y282C, A49W-F276A-K308E, A49W-N227T-F276A, A49W-Y124R-S186R-N227T-F276A-K308E, A49W-N227T-A269C-F276A-Y282C-K308E, A49W-S186R-N227T-A269C-F276A-Y282C, A49W-S186R-N227T-A269C-Y282C-K308E, A49W-S186R-N227T-A269C-F276A-Y282C-K308E, A49W-Y124R-N227T-A269C-F276A-Y282C-K308E, A49W-Y124R-S186R-N227T-A269C-F276A-Y282C, A49W-Y124R-S186R-N227T-A269C-Y282C-K308E, A49W-Y124R-S186R-N227T-A269C-F276A-Y282C-K308E, A49W-S163P-Q215K-N227T-N252P-N267T-F276A-K309E-K312E, A49W-Q215K-N227T-N252P-N267T-F276A-K308E-K309E-K312E, A49W-S163P-Q225G-N227T-N252P-N260Q-S186R-K309E-K312Q, A49W-Q225G-N227T-N252P-N260Q-S186R-K308Q-K309E-K312Q, N59E-S163P, N59E-S186R, N59E-Q215K, N59E-S225G, N59E-N227T, N59E-N252P, N59E-N267T, N59E-F276A, N59E-Y281P, N59E-K308Q, N59E-K308E, N59E-K309E, N59E-K312Q, N59E-K312E, N59E-E232D, N59E-G235W, N59E-N260Q, N59E-H272V, N59E-S279D, N59E-S163P-S186R, N59E-S163P-S225G, N59E-S163P-N227T, N59E-S163P-E232D, N59E-S163P-G235W, N59E-S163P-N252P, N59E-S163P-N260Q, N59E-S163P-H272V, N59E-S163P-S279D, N59E-S163P-Y281P, N59E-S163P-K308Q, N59E-S163P-K309E, N59E-S163P-K312Q, N59E-S186R-S225G, N59E-S186R-N227T, N59E-S186R-E232D, N59E-S186R-G235W, N59E-S186R-N252P, N59E-S186R-N260Q, N59E-S186R-H272V, N59E-S186R-S279D, N59E-S186R-Y281P, N59E-S186R-K308Q, N59E-S186R-K309E, N59E-S186R-K312Q, N59E-S225G-N227T, N59E-S225G-E232D, N59E-S225G-G235W, N59E-S225G-N252P, N59E-S225G-N260Q, N59E-S225G-H272V, N59E-S225G-S279D, N59E-S225G-Y281P, N59E-S225G-K308Q, N59E-S225G-K309E, N59E-S225G-K312Q, N59E-N227T-E232D, N59E-N227T-G235W, N59E-N227T-N252P, N59E-N227T-N260Q, N59E-N227T-H272V, N59E-N227T-S279D, N59E-N227T-Y281P, N59E-N227T-K308Q, N59E-N227T-K309E, N59E-N227T-K312Q, N59E-E232D-G235W, N59E-E232D-N252P, N59E-E232D-N260Q, N59E-E232D-H272V, N59E-E232D-S279D, N59E-E232D-Y281P, N59E-E232D-K308Q, N59E-E232D-K309E, N59E-E232D-K312Q, N59E-G235W-N252P, N59E-G235W-N260Q, N59E-G235W-H272V, N59E-G235W-S279D, N59E-G235W-Y281P, N59E-G235W-K308Q, N59E-G235W-K309E, N59E-G235W-K312Q, N59E-N252P-N260Q, N59E-N252P-H272V, N59E-N252P-S279D, N59E-N252P-Y281P, N59E-N252P-K308Q, N59E-N252P-K309E, N59E-N252P-K312Q, N59E-N260Q-H272V, N59E-N260Q-S279D, N59E-N260Q-Y281P, N59E-N260Q-K308Q, N59E-N260Q-K309E, N59E-N260Q-K312Q, N59E-H272V-S279D, N59E-H272V-Y281P, N59E-H272V-K308Q, N59E-H272V-K309E, N59E-H272V-K312Q, N59E-S279D-Y281P, N59E-S279D-K308Q, N59E-S279D-K309E, N59E-S279D-K312Q, N59E-Y281P-K308Q, N59E-Y281P-K309E, N59E-Y281P-K312Q, N59E-K308Q-K309E, N59E-K308Q-K312Q, N59E-K309E-K312Q, N59E-N227T-N252P-E232D-S225G, N59E-N227T-N252P-E232D-K308Q, N59E-N227T-N252P-E232D-K309E, N59E-N227T-N252P-S225G-K308Q, N59E-N227T-N252P-S225G-K309E, N59E-N227T-N252P-K308Q-K309E, N59E-N227T-E232D-S225G-K308Q, N59E-N227T-E232D-S225G-K309E, N59E-N227T-E232D-K308Q-K309E, N59E-N227T-S225G-K308Q-K309E, N59E-N252P-E232D-S225G-K308Q, N59E-N252P-E232D-S225G-K309E, N59E-N252P-E232D-K308Q-K309E, N59E-N252P-S225G-K308Q-K309E, N59E-E232D-S225G-K308Q-K309E, N59E-N227T-S225G-K308Q-K312Q, N59E-N227T-S225G-K309E-K312Q, N59E-N227T-K308Q-K309E-K312Q, N59E-S225G-K308Q-K309E-K312Q, Y124R-S186R-N227T-F276A-K308E, Y124R-S186R-N227T-A269C-F276A-Y282C-K308E, S163P-S186R, S163P-Q215K, S163P-S225G, S163P-N227T, S163P-N252P, S163P-N267T, S163P-F276A, S163P-Y281P, S163P-K308E, S163P-K308Q, S163P-K309E, S163P-K312Q, S163P-K312E, S163P-E232D, S163P-G235W, S163P-N260Q, S163P-H272V, S163P-S279D, S163P-S186R-S225G, S163P-S186R-N227T, S163P-S186R-E232D, S163P-S186R-G235W, S163P-S186R-N252P, S163P-S186R-N260Q, S163P-S186R-H272V, S163P-S186R-S279D, S163P-S186R-Y281P, S163P-S186R-K308Q, S163P-S186R-K309E, S163P-S186R-K312Q, S163P-S225G-N227T, S163P-S225G-E232D, S163P-S225G-G235W, S163P-S225G-N252P, S163P-S225G-N260Q, S163P-S225G-H272V, S163P-S225G-S279D, S163P-S225G-Y281P, S163P-S225G-K308Q, S163P-S225G-K309E, S163P-S225G-K312Q, S163P-N227T-E232D, S163P-N227T-G235W, S163P-N227T-N252P, S163P-N227T-N260Q, S163P-N227T-H272V, S163P-N227T-S279D, S163P-N227T-Y281P, S163P-N227T-K308Q, S163P-N227T-K309E, S163P-N227T-K312Q, S163P-E232D-G235W, S163P-E232D-N252P, S163P-E232D-N260Q, S163P-E232D-H272V, S163P-E232D-S279D, S163P-E232D-Y281P, S163P-E232D-K308Q, S163P-E232D-K309E, S163P-E232D-K312Q, S163P-G235W-N252P, S163P-G235W-N260Q, S163P-G235W-H272V, S163P-G235W-S279D, S163P-G235W-Y281P, S163P-G235W-K308Q, S163P-G235W-K309E, S163P-G235W-K312Q, S163P-N252P-N260Q, S163P-N252P-H272V, S163P-N252P-S279D, S163P-N252P-Y281P, S163P-N252P-K308Q, S163P-N252P-K309E, S163P-N252P-K312Q, S163P-N260Q-H272V, S163P-N260Q-S279D, S163P-N260Q-Y281P, S163P-N260Q-K308Q, S163P-N260Q-K309E, S163P-N260Q-K312Q, S163P-H272V-S279D, S163P-H272V-Y281P, S163P-H272V-K308Q, S163P-H272V-K309E, S163P-H272V-K312Q, S163P-S279D-Y281P, S163P-S279D-K308Q, S163P-S279D-K309E, S163P-S279D-K312Q, S163P-Y281P-K308Q, S163P-Y281P-K309E, S163P-Y281P-K312Q, S163P-K308Q-K309E, S163P-K308Q-K312Q, S163P-K309E-K312Q, S163P-F276A-K308E, S163P-F276A-K312E, S163P-K308E-K309E, S163P-K308E-K312E, S163P-K309E-K312E, S163P-N227T-F276A, S163P-N227T-N267T, S163P-N252P-F276A, S163P-N252P-K308E, S163P-N252P-K312E, S163P-N252P-N267T, S163P-N267T-K308E, S163P-N267T-K309E, S163P-N267T-K312E, S163P-Q215K-F276A, S163P-Q215K-K308E, S163P-Q215K-K309E, S163P-Q215K-K312E, S163P-Q215K-N227T, S163P Q215K N252P, S163P-Q215K-N252P, S163P-N227T-F276A-K308E, S163P-N227T-S225G-K308Q, S163P-Q215K-N227T-N252P-N267T, S163P-Q215K-N227T-N252P-F276A, S163P-Q215K-N227T-N252P-K308E, S163P-Q215K-N227T-N252P-K309E, S163P-Q215K-N227T-N267T-F276A, S163P-Q215K-N227T-N267T-K308E, S163P-Q215K-N227T-N267T-K309E, S163P-Q215K-N227T-F276A-K308E, S163P-Q215K-N227T-F276A-K309E, S163P-Q215K-N227T-K308E-K309E, S163P-Q215K-N252P-N267T-F276A, S163P-Q215K-N252P-N267T-K308E, S163P-Q215K-N252P-N267T-K309E, S163P-Q215K-N252P-F276A-K308E, S163P-Q215K-N252P-F276A-K309E, S163P-Q215K-N252P-K308E-K309E, S163P-Q215K-N267T-F276A-K308E, S163P-Q215K-N267T-F276A-K309E, S163P-Q215K-N267T-K308E-K309E, S163P-Q215K-F276A-K308E-K309E, S163P-N227T-N252P-N267T-F276A, S163P-N227T-N252P-N267T-K308E, S163P-N227T-N252P-N267T-K309E, S163P-N227T-N252P-F276A-K308E, S163P-N227T-N252P-F276A-K309E, S163P-N227T-N252P-K308E-K309E, S163P-N227T-N267T-F276A-K308E, S163P-N227T-N267T-F276A-K309E, S163P-N227T-N267T-K308E-K309E, S163P-N227T-F276A-K308E-K309E, S163P-N252P-N267T-F276A-K308E, S163P-N252P-N267T-F276A-K309E, S163P-N252P-N267T-K308E-K309E, S163P-N252P-F276A-K308E-K309E, S163P-N267T-F276A-K308E-K309E, S163P-N59E-N227T-N252P-E232D, S163P-N59E-N227T-N252P-S225G, S163P-N59E-N227T-N252P-K308Q, S163P-N59E-N227T-N252P-K309E, S163P-N59E-N227T-E232D-S225G, S163P-N59E-N227T-E232D-K308Q, S163P-N59E-N227T-E232D-K309E, S163P-N59E-N227T-S225G-K308Q, S163P-N59E-N227T-S225G-K309E, S163P-N59E-N227T-K308Q-K309E, S163P-N59E-N252P-E232D-S225G, S163P-N59E-N252P-E232D-K308Q, S163P-N59E-N252P-E232D-K309E, S163P-N59E-N252P-S225G-K308Q, S163P-N59E-N252P-S225G-K309E, S163P-N59E-N252P-K308Q-K309E, S163P-N59E-E232D-S225G-K308Q, S163P-N59E-E232D-S225G-K309E, S163P-N59E-E232D-K308Q-K309E, S163P-N59E-S225G-K308Q-K309E, S163P-N227T-N252P-E232D-S225G, S163P-N227T-N252P-E232D-K308Q, S163P-N227T-N252P-E232D-K309E, S163P-N227T-N252P-S225G-K308Q, S163P-N227T-N252P-S225G-K309E, S163P-N227T-N252P-K308Q-K309E, S163P-N227T-E232D-S225G-K308Q, S163P-N227T-E232D-S225G-K309E, S163P-N227T-E232D-K308Q-K309E, S163P-N227T-S225G-K308Q-K309E, S163P-N252P-E232D-S225G-K308Q, S163P-N252P-E232D-S225G-K309E, S163P-N252P-E232D-K308Q-K309E, S163P-N252P-S225G-K308Q-K309E, S163P-E232D-S225G-K308Q-K309E, S163P-N227T-F276A-K308E-K312E, S163P-N227T-S225G-K308Q-K312Q, N227T-A269C-F276A-Y282C-K308E, S163P-N227T-F276A-K308E-K309E-K312E, S163P-N227T-S186R-K308Q-K309E-K312Q, S163P-Q215K-N227T-N252P-N267T-F276A, S163P-N227T-N252P-F276A-K308E-K309E-K312E, S163P-N227T-N267T-F276A-K308E-K309E-K312E, S163P-Q215K-N227T-F276A-K308E-K309E-K312E, S163P-N227T-S225G-S186R-K308Q-K309E-K312Q, S163P-S186R-N227T-S186R-K308Q-K309E-K312Q, S163P-Q215K-N227T-N252P-N267T-F276A-K308E, S163P-Q215K-N227T-N252P-N267T-F276A-K309E, S163P-Q215K-N227T-N252P-N267T-F276A-K312E, S163P-Q215K-N227T-N252P-N267T-F276A-K308E-K309E, S163P-Q215K-N227T-N252P-N267T-F276A-K308E-K312E, S163P-Q215K-N227T-N252P-N267T-F276A-K309E-K312E, S163P-Q215K-N227T-N252P-N267T-K308E-K309E-K312E, S163P-Q215K-N227T-N252P-F276A-K308E-K309E-K312E, S163P-Q215K-N227T-N267T-F276A-K308E-K309E-K312E, S163P-Q215K-N252P-N267T-F276A-K308E-K309E-K312E, S163P-N227T-N252P-N267T-F276A-K308E-K309E-K312E, S163P-G235W-N227T-N252P-S186R-N260Q-K308Q-K309E, S163P-G235W-N227T-N252P-S186R-N260Q-K308Q-K312Q, S163P-G235W-N227T-N252P-S186R-N260Q-K309E-K312Q, S163P-G235W-N227T-N252P-S186R-K308Q-K309E-K312Q, S163P-G235W-N227T-N252P-N260Q-K308Q-K309E-K312Q, S163P-G235W-N227T-S186R-N260Q-K308Q-K309E-K312Q, S163P-G235W-N252P-S186R-N260Q-K308Q-K309E-K312Q, S163P-N227T-N252P-S186R-N260Q-K308Q-K309E-K312Q, S163P-N227T-N252P-S186R-N260Q-K308E-K309E-K312E, S163P-S225G-N227T-N252P-N260Q-K308E-K309E-K312E, S163P-S225G-N227T-S186R-N260Q-K308E-K309E-K312E, S163P-Q215K-N227T-N252P-N267T-F276A-K308E-K309E-K312E, S163P-Q225G-N227T-N252P-N260Q-S186R-K308Q-K309E-K312Q, S163P-S186R-S225G-N227T-G235W-N252P-K308Q-K309E-K312Q, S163P-Q215K-N227T-N252P-N267T-F276A-K308Q-K309E-K312E, S163P-Q215K-N227T-N252P-N267T-F276A-K308E-K309E-K312Q, S186R-Q215K, S186R-S225G, S186R-N227T, S186R-N252P, S186R-N267T, S186R-F276A, S186R-Y281P, S186R-K308E, S186R-K308Q, S186R-K309E, S186R-K312Q, S186R-K312E, S186R-E232D, S186R-G235W, S186R-N260Q, S186R-H272V, S186R-S279D, S186R-S225G-N227T, S186R-S225G-E232D, S186R-S225G-G235W, S186R-S225G-N252P, S186R-S225G-N260Q, S186R-S225G-H272V, S186R-S225G-S279D, S186R-S225G-Y281P, S186R-S225G-K308Q, S186R-S225G-K309E, S186R-S225G-K312Q, S186R-N227T-E232D, S186R-N227T-G235W, S186R-N227T-N252P, S186R-N227T-N260Q, S186R-N227T-H272V, S186R-N227T-S279D, S186R-N227T-Y281P, S186R-N227T-K308Q, S186R-N227T-K309E, S186R-N227T-K312Q, S186R-E232D-G235W, S186R-E232D-N252P, S186R-E232D-N260Q, S186R-E232D-H272V, S186R-E232D-S279D, S186R-E232D-Y281P, S186R-E232D-K308Q, S186R-E232D-K309E, S186R-E232D-K312Q, S186R-G235W-N252P, S186R-G235W-N260Q, S186R-G235W-H272V, S186R-G235W-S279D, S186R-G235W-Y281P, S186R-G235W-K308Q, S186R-G235W-K309E, S186R-G235W-K312Q, S186R-N252P-N260Q, S186R-N252P-H272V, S186R-N252P-S279D, S186R-N252P-Y281P, S186R-N252P-K308Q, S186R-N252P-K309E, S186R-N252P-K312Q, S186R-N260Q-H272V, S186R-N260Q-S279D, S186R-N260Q-Y281P, S186R-N260Q-K308Q, S186R-N260Q-K309E, S186R-N260Q-K312Q, S186R-H272V-S279D, S186R-H272V-Y281P, S186R-H272V-K308Q, S186R-H272V-K309E, S186R-H272V-K312Q, S186R-S279D-Y281P, S186R-S279D-K308Q, S186R-S279D-K309E, S186R-S279D-K312Q, S186R-Y281P-K308Q, S186R-Y281P-K309E, S186R-Y281P-K312Q, S186R-K308Q-K309E, S186R-K308Q-K312Q, S186R-K309E-K312Q, S186R-F276A-K308E, S186R-N227T-F276A, S186R-N227T-K308E, S186R-N227T-E232D-K308E, S186R-A269C-F276A-Y282C-K308E, S186R-N227T-A269C-F276A-Y282C, S186R-N227T-A269C-Y282C-K308E, S186R-N227T-A269C-F276A-Y282C-K308E, S186R-N227T-N252P-S186R-K308Q-K309E-K312Q, S186R-N227T-S225G-S186R-K308Q-K309E-K312Q, S163P-N227T-N252P-S186R-K308Q-K309E-K312Q, Q215K-S225G, Q215K-N227T, Q215K-N252P, Q215K-N267T, Q215K-F276A, Q215K-Y281P, Q215K-K308E, Q215K-K308Q, Q215K-K309E, Q215K-K312Q, Q215K-K312E, Q215K-F276A-K308E, Q215K-F276A-K312E, Q215K-K308E-K312E, Q215K-K309E-K312E, Q215K-N227T-F276A, Q215K-N227T-K308E, Q215K-N227T-K309E, Q215K-N227T-K312E, Q215K-N227T-N252P, Q215K-N227T-N267T, Q215K-N252P-F276A, Q215K-N252P-K309E, Q215K-N252P-K312E, Q215K-N252P-N267T, Q215K-N267T-F276A, Q215K-N267T-K312E, Q215K-N227T-N252P-N267T-F276A, Q215K-N227T-N252P-N267T-K308E, Q215K-N227T-N252P-N267T-K309E, Q215K-N227T-N252P-F276A-K308E, Q215K-N227T-N252P-F276A-K309E, Q215K-N227T-N252P-K308E-K309E, Q215K-N227T-N267T-F276A-K308E, Q215K-N227T-N267T-F276A-K309E, Q215K-N227T-N267T-K308E-K309E, Q215K-N227T-F276A-K308E-K309E, Q215K-N252P-N267T-F276A-K308E, Q215K-N252P-N267T-F276A-K309E, Q215K-N252P-N267T-K308E-K309E, Q215K-N252P-F276A-K308E-K309E, Q215K-N267T-F276A-K308E-K309E, Q215K-N227T-F276A-K308E-K312E, Q215K-N227T-F276A-K309E-K312E, Q215K-N227T-K308E-K309E-K312E, Q215K-F276A-K308E-K309E-K312E, Q215K-N227T-F276A-K308E-K309E-K312E, Q215K-N227T-N252P-F276A-K308E-K309E-K312E, Q215K-N227T-N267T-F276A-K308E-K309E-K312E, Q215K-N227T-N252P-N267T-F276A-K308E-K309E-K312E, S225G-N227T, S225G-N252P, S225G-N267T, S225G-F276A, S225G-Y281P, S225G-K308E, S225G-K308Q, S225G-K309E, S225G-K312Q, S225G-K312E, S225G-E232D, S225G-G235W, S225G-N260Q, S225G-H272V, S225G-S279D, S225G-N227T-E232D, S225G-N227T-G235W, S225G-N227T-N252P, S225G-N227T-N260Q, S225G-N227T-H272V, S225G-N227T-S279D, S225G-N227T-Y281P, S225G-N227T-K308Q, S225G-N227T-K309E, S225G-N227T-K312Q, S225G-E232D-G235W, S225G-E232D-N252P, S225G-E232D-N260Q, S225G-E232D-H272V, S225G-E232D-S279D, S225G-E232D-Y281P, S225G-E232D-K308Q, S225G-E232D-K309E, S225G-E232D-K312Q, S225G-G235W-N252P, S225G-G235W-N260Q, S225G-G235W-H272V, S225G-G235W-S279D, S225G-G235W-Y281P, S225G-G235W-K308Q, S225G-G235W-K309E, S225G-G235W-K312Q, S225G-N252P-N260Q, S225G-N252P-H272V, S225G-N252P-S279D, S225G-N252P-Y281P, S225G-N252P-K308Q, S225G-N252P-K309E, S225G-N252P-K312Q, S225G-N260Q-H272V, S225G-N260Q-S279D, S225G-N260Q-Y281P, S225G-N260Q-K308Q, S225G-N260Q-K309E, S225G-N260Q-K312Q, S225G-H272V-S279D, S225G-H272V-Y281P, S225G-H272V-K308Q, S225G-H272V-K309E, S225G-H272V-K312Q, S225G-S279D-Y281P, S225G-S279D-K308Q, S225G-S279D-K309E, S225G-S279D-K312Q, S225G-Y281P-K308Q, S225G-Y281P-K309E, S225G-Y281P-K312Q, S225G-K308Q-K309E, S225G-K308Q-K312Q, S225G-K309E-K312Q, S225G-K308Q-K309E-K312Q, S225G-N227T-N252P-S186R-N260Q-K308E-K309E-K312E, N227T-N252P, N227T-N267T, N227T-F276A, N227T-Y281P, N227T-K308Q, N227T-K308E, N227T-K309E, N227T-K312Q, N227T-K312E, N227T-E232D, N227T-G235W, N227T-N260Q, N227T-H272V, N227T-S279D, N227T-E232D-G235W, N227T-E232D-N252P, N227T-E232D-N260Q, N227T-E232D-H272V, N227T-E232D-S279D, N227T-E232D-Y281P, N227T-E232D-K308Q, N227T-E232D-K309E, N227T-E232D-K312Q, N227T-G235W-N252P, N227T-G235W-N260Q, N227T-G235W-H272V, N227T-G235W-S279D, N227T-G235W-Y281P, N227T-G235W-K308Q, N227T-G235W-K309E, N227T-G235W-K312Q, N227T-N252P-N260Q, N227T-N252P-H272V, N227T-N252P-S279D, N227T-N252P-Y281P, N227T-N252P-K308Q, N227T-N252P-K309E, N227T-N252P-K312Q, N227T-N260Q-H272V, N227T-N260Q-S279D, N227T-N260Q-Y281P, N227T-N260Q-K308Q, N227T-N260Q-K309E, N227T-N260Q-K312Q, N227T-H272V-S279D, N227T-H272V-Y281P, N227T-H272V-K308Q, N227T-H272V-K309E, N227T-H272V-K312Q, N227T-S279D-Y281P, N227T-S279D-K308Q, N227T-S279D-K309E, N227T-S279D-K312Q, N227T-Y281P-K308Q, N227T-Y281P-K309E, N227T-Y281P-K312Q, N227T-K308Q-K309E, N227T-K308Q-K312Q, N227T-K309E-K312Q, N227T-N252P-K308E, N227T-N252P-K312E, N227T-N252P-N267T, N227T-N267T-F276A, N227T-N267T-K308E, N227T-N267T-K309E, N227T-N267T-K312E, N227T-F276A-K308E, N227T-F276A-K309E, N227T-F276A-K312E, N227T-K308E-K309E, N227T-K308E-K312E, N227T-K309E-K312E, N227T-F276A-K308E-K309E, N227T-F276A-K308E-K312E, N227T-F276A-K309E-K312E, N227T-K308E-K309E-K312E, N227T-S225G-K308Q-K309E, N227T-S225G-K308Q-K312Q, N227T-S225G-K309E-K312Q, N227T-K308Q-K309E-K312Q, N227T-N267T-F276A-K308E, N227T-N267T-F276A-K309E, N227T-N267T-F276A-K312E, N227T-N267T-S225G-K308Q, N227T-N267T-S225G-K309E, N227T-N267T-S225G-K312Q, N227T-N252P-N267T-F276A-K308E, N227T-N252P-N267T-F276A-K309E, N227T-N252P-N267T-K308E-K309E, N227T-N252P-F276A-K308E-K309E, N227T-N267T-F276A-K308E-K309E, N227T-F276A-K308E-K309E-K312E, N227T-N252P-E232D-S225G-K308Q, N227T-N252P-E232D-S225G-K309E, N227T-N252P-E232D-K308Q-K309E, N227T-N252P-S225G-K308Q-K309E, N227T-E232D-S225G-K308Q-K309E, N227T-S225G-K308Q-K309E-K312Q, N227T-N267T-F276A-K308E-K312E, N227T-N267T-F276A-K309E-K312E, N227T-E232D-S225G-K308Q-K312Q, N227T-E232D-S225G-K309E-K312Q, N227T-N267T-F276A-K308E-K309E-K312E, N227T-N252P-F276A-K308E-K309E-K312E, N227T-S225G-S186R-K308Q-K309E-K312Q, N227T-N252P-S186R-K308Q-K309E-K312Q, N227T-N252P-N267T-F276A-K308E-K309E-K312E, N227T-N252P-S225G-S186R-K308Q-K309E-K312Q, E232D-G235W, E232D-N252P, E232D-N260Q, E232D-H272V, E232D-S279D, E232D-Y281P, E232D-K308Q, E232D-K309E, E232D-K312Q, E232D-G235W-N252P, E232D-G235W-N260Q, E232D-G235W-H272V, E232D-G235W-S279D, E232D-G235W-Y281P, E232D-G235W-K308Q, E232D-G235W-K309E, E232D-G235W-K312Q, E232D-N252P-N260Q, E232D-N252P-H272V, E232D-N252P-S279D, E232D-N252P-Y281P, E232D-N252P-K308Q, E232D-N252P-K309E, E232D-N252P-K312Q, E232D-N260Q-H272V, E232D-N260Q-S279D, E232D-N260Q-Y281P, E232D-N260Q-K308Q, E232D-N260Q-K309E, E232D-N260Q-K312Q, E232D-H272V-S279D, E232D-H272V-Y281P, E232D-H272V-K308Q, E232D-H272V-K309E, E232D-H272V-K312Q, E232D-S279D-Y281P, E232D-S279D-K308Q, E232D-S279D-K309E, E232D-S279D-K312Q, E232D-Y281P-K308Q, E232D-Y281P-K309E, E232D-Y281P-K312Q, E232D-K308Q-K309E, E232D-K308Q-K312Q, E232D-K309E-K312Q, G235W-N252P, G235W-N260Q, G235W-H272V, G235W-S279D, G235W-Y281P, G235W-K308Q, G235W-K309E, G235W-K312Q, G235W-N252P-N260Q, G235W-N252P-H272V, G235W-N252P-S279D, G235W-N252P-Y281P, G235W-N252P-K308Q, G235W-N252P-K309E, G235W-N252P-K312Q, G235W-N260Q-H272V, G235W-N260Q-S279D, G235W-N260Q-Y281P, G235W-N260Q-K308Q, G235W-N260Q-K309E, G235W-N260Q-K312Q, G235W-H272V-S279D, G235W-H272V-Y281P, G235W-H272V-K308Q, G235W-H272V-K309E, G235W-H272V-K312Q, G235W-S279D-Y281P, G235W-S279D-K308Q, G235W-S279D-K309E, G235W-S279D-K312Q, G235W-Y281P-K308Q, G235W-Y281P-K309E, G235W-Y281P-K312Q, G235W-K308Q-K309E, G235W-K308Q-K312Q, G235W-K309E-K312Q, G235W-N227T-S186R-K308Q-K309E-K312Q, G235W-N227T-N252P-S186R-N260Q-K308Q-K309E-K312Q, N252P-N267T, N252P-F276A, N252P-Y281P, N252P-K308Q, N252P-K308E, N252P-K309E, N252P-K312Q, N252P-K312E, N252P-N260Q, N252P-H272V, N252P-S279D, N252P-N260Q-H272V, N252P-N260Q-S279D, N252P-N260Q-Y281P, N252P-N260Q-K308Q, N252P-N260Q-K309E, N252P-N260Q-K312Q, N252P-H272V-S279D, N252P-H272V-Y281P, N252P-H272V-K308Q, N252P-H272V-K309E, N252P-H272V-K312Q, N252P-S279D-Y281P, N252P-S279D-K308Q, N252P-S279D-K309E, N252P-S279D-K312Q, N252P-Y281P-K308Q, N252P-Y281P-K309E, N252P-Y281P-K312Q, N252P-K308Q-K309E, N252P-K308Q-K312Q, N252P-K309E-K312Q, N252P-F276A-K308E, N252P-F276A-K309E, N252P-F276A-K312E, N252P-K308E-K309E, N252P-K308E-K312E, N252P-N267T-F276A, N252P-N267T-K308E, N252P-N267T-K309E, N252P-N267T-K312E, N252P-N267T-F276A-K308E-K309E, N252P-E232D-S225G-K308Q-K309E, N260Q-H272V, N260Q-S279D, N260Q-Y281P, N260Q-K308Q, N260Q-K309E, N260Q-K312Q, N260Q-H272V-S279D, N260Q-H272V-Y281P, N260Q-H272V-K308Q, N260Q-H272V-K309E, N260Q-H272V-K312Q, N260Q-S279D-Y281P, N260Q-S279D-K308Q, N260Q-S279D-K309E, N260Q-S279D-K312Q, N260Q-Y281P-K308Q, N260Q-Y281P-K309E, N260Q-Y281P-K312Q, N260Q-K308Q-K309E, N260Q-K308Q-K312Q, N260Q-K309E-K312Q, N267T-F276A, N267T-Y281P, N267T-K308E, N267T-K309E, N267T-K312Q, N267T-K312E, N267T-F276A-K308E, N267T-F276A-K309E, N267T-F276A-K312E, N267T-K308E-K309E, N267T-K308E-K312E, N267T-K309E-K312E, H272V-S279D, H272V-Y281P, H272V-K308Q, H272V-K309E, H272V-K312Q, H272V-S279D-Y281P, H272V-S279D-K308Q, H272V-S279D-K309E, H272V-S279D-K312Q, H272V-Y281P-K308Q, H272V-Y281P-K309E, H272V-Y281P-K312Q, H272V-K308Q-K309E, H272V-K308Q-K312Q, H272V-K309E-K312Q, F276A-Y281P, F276A-K308Q, F276A-K308E, F276A-K309E, F276A-K312Q, F276A-K312E, F276A-K308E-K309E, F276A-K308E-K312E, F276A-K309E-K312E, F276A-K308E-K309E-K312E, S279D-Y281P, S279D-K308Q, S279D-K309E, S279D-K312Q, S279D-Y281P-K308Q, S279D-Y281P-K309E, S279D-Y281P-K312Q, S279D-K308Q-K309E, S279D-K308Q-K312Q, S279D-K309E-K312Q, Y281P-K308Q, Y281P-K308E, Y281P-K309E, Y281P-K312Q, Y281P-K312E, Y281P-K308Q-K309E, Y281P-K308Q-K312Q, Y281P-K309E-K312Q, K308E-K312Q, K308E-K312E, K308Q-K312Q, K309E-K312Q, K309E-K312E, K308E-K309E, K308Q-K309E, K308Q-K309E-K312Q, K308E-K309E-K312E, K312E-N227T-F276A, K312E-N227T-K308E, K312E-N227T-K309E, K312E-N227T-K312E, K312E-F276A-K308E, K312E-F276A-K309E, K312E-F276A-K312E, K312E-K308E-K309E, K312E-K308E-K312E, K312E-K309E-K312E, K312E-N227T-F276A-K308E, K312E-N227T-F276A-K309E, K312E-N227T-F276A-K312E, K312E-N227T-K308E-K309E, K312E-N227T-K308E-K312E, K312E-N227T-K309E-K312E, K312E-F276A-K308E-K309E, K312E-F276A-K308E-K312E, K312E-F276A-K309E-K312E, K312E-K308E-K309E-K312E, K312Q-N227T-S225G-K308Q, K312Q-N227T-S225G-K309E, K312Q-N227T-S225G-K312Q, K312Q-N227T-K308Q-K309E, K312Q-N227T-K308Q-K312Q, K312Q-N227T-K309E-K312Q, K312Q-S225G-K308Q-K309E, K312Q-S225G-K308Q-K312Q, K312Q-S225G-K309E-K312Q and K312Q-K308Q-K309E-K312Q, based on the numbering according to SEQ ID NO:1.

In further preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96% or 96.5%, and has at least ten and increasingly preferably eleven, twelve, 13, 14, 15, 16, 17, 18 or more amino acid substitutions, wherein the dispersin has an amino acid substitution combination which is selected from the group consisting of Q3F-A49W-N59E-S163P-S186R-S225G-N227T-N252P-F276A-Y281P-K308E-K309E-K312E, Q3F-A49W-N59E-S163P-S186R-N227T-E232D-N252P-F276A-S279G-K308E-K309E-K312E, Q3F-T17W-A49W-N59E-S163P-S186R-S225G-N227T-G235W-N252P-H272P-Y281P-K308Q-K309E-K312Q, Q3F-A49W-N59E-S163P-S186R-S225G-N227T-N252P-F276A-S279G-K308E-K309E-K312E, Q3F-A49W-N59E-S163P-S186R-S225G-N227T-E232D-N252P-F276A-S279G-K308E-K309E-K312E, Q3F-S163P-S186R-Q215K-N227T-E232D-N252P-N267T-F276A-S279G-K308E-K309E-K312E, Q3F-S163P-S186R-Q215K-S225G-N227T-N252P-N267T-F276A-S279G-K308E-K309E-K312E, Q3F-A49W-N59E-S163P-S186R-N227T-E232D-N252P-F276A-S279G-Y281P-K308E-K309E-K312E, Q3F-T17W-A49W-N59E-S163P-S186R-D207N-T218Q-S225G-N227T-G235W-N252P-H272P-Y281P-K308Q-K309E-K312Q, Q3F-A49W-N59E-S163P-S186R-S225G-N227T-N252P-F276A-S279G-Y281P-K308E-K309E-K312E, Q3F-A49W-N59E-S163P-S186R-S225G-N227T-E232D-N252P-H272V-F276A-S279G-K308E-K309E-K312E, Q3F-H15Y-V140I-S163P-S186R-Q215K-S225G-N227T-E232D-G235W-N252P-N260Q-N267T-H272V-S279D-Y281P-K308Q-K309E-K312Q, Q3F-H15Y-S163P-S186R-Q215K-S225G-N227T-E232D-N252P-N260Q-N267T-H272V-S279D-Y281P-K308Q-K309E-K312Q, Q3F-H15Y-T17W-A49W-N59E-S163P-S186R-T218Q-S225G-N227T-G235W-S237W-N252P-H272P-K308Q-K309E-K312Q, Q3F-H15Y-T17W-A49W-N59E-S163P-S186R-D207N-T218Q-S225G-N227T-G235W-S237W-N252P-H272P-Y281P-K308Q-K309E-K312Q, Q3F-S163P-S186R-Q215K-N227T-E232D-N252P-N267T-H272V-F276A-S279G-K308E-K309E-K312E, Q3F-S163P-S186R-Q215K-N227T-E232D-N252P-N267T-F276A-S279G-Y281P-K308E-K309E-K312E, Q3F-S163P-Q215K-N227T-E232D-N252P-N267T-F276A-S279G-Y281P-K308E-K309E-K312E, Q3F-H15Y-T17W-A49W-N59E-S163P-S186R-T218Q-S225G-N227T-G235W-S237W-N252P-H272P-Y281P-K308Q-K309E-K312Q, Q3F-S163P-Q215K-S225G-N227T-E232D-N252P-N267T-H272V-F276A-S279G-K308E-K309E-K312E, Q3F-A49W-N59E-S163P-S186R-N227T-E232D-N252P-H272V-F276A-S279G-Y281P-K308E-K309E-K312E, Q3F-S163P-S186R-Q215K-S225G-N227T-N252P-N267T-F276A-S279G-Y281P-K308E-K309E-K312E, Q3F-S163P-Q215K-N227T-E232D-N252P-N267T-H272V-F276A-S279G-Y281P-K308E-K309E-K312E, Q3F-A49W-N59E-S163P-S186R-S225G-N227T-E232D-N252P-F276A-S279G-Y281P-K308E-K309E-K312E, Q3F-A49W-N59E-S163P-S186R-S225G-N227T-N252P-H272V-F276A-S279G-Y281P-K308E-K309E-K312E, Q3F-S163P-S186R-Q215K-S225G-N227T-E232D-N252P-N260Q-N267T-H272P-S279D-Y281P-K308Q-K309E-K312Q, Q3F-S163P-S186R-Q215K-N227T-E232D-N252P-N267T-H272V-F276A-S279G-Y281P-K308E-K309E-K312E, Q3F-S163P-S186R-Q215K-S225G-N227T-N252P-N267T-H272V-F276A-S279G-Y281P-K308E-K309E-K312E, Q3F-T17W-S163P-S186R-Q215K-S225G-N227T-E232D-G235W-N252P-N267T-H272V-F276A-S279G-Y281P-K308E-K309E-K312Q, Q3F-S163P-S186R-Q215K-S225G-N227T-E232D-N252P-N267T-H272V-F276A-S279G-Y281P-K308E-K309E-K312Q, Q3F-A49W-N59E-S163P-S186R-S225G-N227T-E232D-N252P-H272V-F276A-S279G-Y281P-K308E-K309E-K312E, Q3F-S163P-S186R-Q215K-S225G-N227T-E232D-N252P-N267T-H272V-F276A-S279G-Y281P-K308E-K309E-K312E, Q3F-H15Y-T17W-S163P-S186R-Q215K-S225G-N227T-E232D-G235W-N252P-N260Q-N267T-H272V-S279D-Y281P-K308Q-K309E-K312Q, Q3F-S163P-Q215K-S225G-N227T-E232D-N252P-N267T-H272V-F276A-S279G-Y281P-K308E-K309E-K312E, Q3F-H15Y-S163P-S186R-Q215K-S225G-N227T-E232D-G235W-N252P-N260Q-N267T-H272P-S279D-Y281P-K308Q-K309E-K312Q, Q3F-A49W-N59E-S163P-S186R-S225G-N227T-E232D-G235W-N252P-N260Q-H272P-F276A-S279D-Y281P-K308E-K309E-K312Q, Q3F-H15Y-T17W-S163P-S186R-Q215K-S225G-N227T-E232D-N252P-N260Q-N267T-H272V-S279D-Y281P-K308Q-K309E-K312Q, Q3F-H15Y-S163P-S186R-Q215K-S225G-N227T-E232D-N252P-N260Q-N267T-H272P-S279D-Y281P-K308Q-K309E-K312Q, Q3F-H15Y-S163P-S186R-Q215K-S225G-N227T-E232D-G235W-N252P-N267T-H272V-F276A-S279G-Y281P-K308Q-K309E-K312Q, Q3F-H15Y-T17W-S163P-S186R-Q215K-S225G-N227T-E232D-N252P-N267T-H272V-F276A-S279G-Y281P-K308Q-K309E-K312Q, Q3F-H15Y-A49W-N59E-V140I-S163P-S186R-Q215K-S225G-N227T-E232D-G235W-N252P-N260Q-N267T-H272V-S279D-Y281P-K308Q-K309E-K312Q, Q3I-A49W-N59E-S163P-S186R-S225G-N227T-Y281P-K308Q-K309E-K312Q, Q3I-A49W-N59E-S163P-S186R-S225G-E232D-Y281P-K308Q-K309E-K312Q, Q3I-A49W-N59E-S163P-S186R-S225G-G235W-Y281P-K308Q-K309E-K312Q, Q3I-A49W-N59E-S163P-S186R-S225G-N252P-Y281P-K308Q-K309E-K312Q, Q3I-A49W-N59E-S163P-S186R-S225G-N260Q-Y281P-K308Q-K309E-K312Q, Q3I-A49W-N59E-S163P-S186R-S225G-N227T-H272V-Y281P-K308Q-K309E, Q3I-A49W-N59E-S163P-S186R-S225G-N227T-S279D-Y281P-K308Q-K312Q, Q3I-H15Y-A49W-N59E-S163P-S186R-S225G-N227T-E232D-N252P-N260Q-N267T-H272V-S279D-Y281P-K308Q-K309E-K312Q, Q3I-H15Y-A49W-N59E-S163P-S186R-S225G-N227T-E232D-N252P-N260Q-N267T-H272P-S279D-Y281P-K308Q-K309E-K312Q, Q3I-H15Y-A49W-N59E-S163P-S186R-S225G-N227T-E232D-G235W-N252P-N260Q-H272V-S279D-Y281P-K308Q-K309E-K312E, Q3I-H15Y-A49W-N59E-S163P-S186R-S225G-N227T-E232D-G235W-N252P-N260Q-H272V-S279D-Y281P-K308E-K309E-K312Q, Q3I-H15Y-A49W-N59E-S163P-S186R-S225G-N227T-E232D-G235W-N252P-N260Q-H272V-S279D-Y281P-K308Q-K309E-K312Q, H15Y-A49W-N59E-S163P-S186R-S225G-N227T-E232D-Y281P-K309E-K312Q, H15Y-A49W-N59E-S163P-S186R-S225G-N227T-G235W-K308Q-K309E-K312Q, H15Y-A49W-S163P-S186R-S225G-N227T-Y281P-S288P-K308Q-K309E-K312Q, H15Y-A49W-S163P-S186R-S225G-N227T-N252P-Y281P-K308Q-K309E-K312Q, H15Y-A49W-S163P-S186R-S225G-N227T-H272P-Y281P-K308Q-K309E-K312Q, H15Y-A49W-S163P-S186R-S225G-N227T-S279D-Y281P-K308Q-K309E-K312Q, H15Y-A49W-N59E-S163P-S186R-S225G-N227T-E232D-Y281P-K308Q-K312Q, H15Y-A49W-N59E-S163P-S186R-S225G-N227T-E232D-G235W-N252P-N260Q-H272P-S279D-Y281P-K308Q-K309E-K312Q, H15Y-A49W-N59E-S163P-S186R-S225G-N227T-E232D-G235W-N252P-N260Q-H272V-S279D-Y281P-K308Q-K309E-K312Q, H15Y-A49W-N59E-S163P-S186R-S225G-N227T-E232D-N252P-N260Q-H272V-Y281P-K308Q-K309E-K312Q, H15Y-A49W-N59E-S163P-S186R-S225G-N227T-E232D-G235W-N252P-N260Q-H272V-S279D-Y281P-K309E-K312Q, H15Y-A49W-N59E-S163P-S186R-S225G-N227T-E232D-N252P-N260Q-H272P-S279D-Y281P-S288P-K308Q-K309E-K312Q, H15Y-A49W-N59E-S163P-S186R-S225G-N227T-E232D-N252P-N260Q-H272P-S279D-Y281P-K308Q-K309E-K312Q, H15Y-T17W-A49W-N59E-S163P-S186R-S225G-N227T-E232D-G235W-N252P-N260Q-H272V-S279D-Y281P-K308Q-K309E-K312Q, H15Y-T17W-A49W-N59E-S163P-S186R-S225G-N227T-E232D-N252P-N260Q-H272V-S279D-Y281P-K308Q-K309E-K312Q, A49W-N59E-S163P-S186R-S225G-N227T-E232D-N252P-F276A-S279G-K308E-K309E-K312E, A49W-N59E-S163P-S186R-N227T-E232D-N252P-H272V-F276A-S279G-K308E-K309E-K312E, A49W-N59E-S163P-S186R-N227T-E232D-N252P-F276A-S279G-Y281P-K308E-K309E-K312E, A49W-N59E-S163P-S186R-S225G-N227T-N252P-F276A-S279G-Y281P-K308E-K309E-K312E, A49W-N59E-S163P-S186R-S225G-N227T-E232D-N252P-H272V-F276A-S279G-K308E-K309E-K312E, A49W-N59E-S163P-S186R-S225G-N227T-E232D-N252P-F276A-S279G-Y281P-K308E-K309E-K312E, A49W-N59E-S163P-S186R-N227T-E232D-N252P-H272V-F276A-S279G-Y281P-K308E-K309E-K312E, A49W-N59E-S163P-S186R-S225G-N227T-N252P-H272V-F276A-S279G-Y281P-K308E-K309E-K312E, A49W-N59E-S163P-S186R-S225G-N227T-E232D-N252P-H272V-F276A-S279G-Y281P-K308E-K309E-K312E, S163P-S186R-Q215K-N227T-E232D-N252P-N267T-F276A-S279G-Y281P-K308E-K309E-K312E, S163P-S186R-S225G-N227T-E232D-N252P-N267T-F276A-S279G-Y281P-K308E-K309E-K312E, S163P-S186R-Q215K-S225G-N227T-E232D-N252P-N267T-F276A-S279G-Y281P-K308E-K309E-K312E, S163P-Q215K-S225G-N227T-E232D-N252P-N267T-F276A-S279G-Y281P-K308E-K309E-K312E, S163P-S186R-Q215K-S225G-N227T-E232D-N252P-N267T-H272V-F276A-S279G-K308E-K309E-K312E, S163P-Q215K-S225G-N227T-E232D-N252P-N267T-H272V-F276A-S279G-K308E-K309E-K312E, S163P-Q215K-N227T-E232D-N252P-N267T-H272V-F276A-S279G-Y281P-K308E-K309E-K312E, S163P-S186R-Q215K-S225G-N227T-N252P-N267T-H272V-F276A-S279G-Y281P-K308E-K309E-K312E, S163P-Q215K-S225G-N227T-N252P-N267T-H272V-F276A-S279G-Y281P-K308E-K309E-K312E, S163P-Q215K-S225G-N227T-E232D-N252P-N267T-H272V-F276A-S279G-Y281P-K308E-K309E-K312E and S163P-S186R-Q215K-S225G-N227T-E232D-N252P-N267T-H272V-F276A-S279G-Y281P-K308E-K309E-K312E, based on the numbering according to SEQ ID NO:1.

In particularly preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94% or 95%, and has at least 15 and increasingly preferably 16, 17, 18 or 19 amino acid substitutions, wherein the dispersin has an amino acid substitution combination selected from the group consisting of Q3F-T17W-A49W-N59E-S163P-S186R-S225G-N227T-G235W-N252P-H272P-Y281P-K308Q-K309E-K312Q, Q3F-T17W-A49W-N59E-S163P-S186R-D207N-T218Q-S225G-N227T-G235W-N252P-H272P-Y281P-K308Q-K309E-K312Q, Q3F-A49W-N59E-S163P-S186R-S225G-N227T-E232D-N252P-H272V-F276A-S279G-K308E-K309E-K312E, Q3F-H15Y-V140I-S163P-S186R-Q215K-S225G-N227T-E232D-G235W-N252P-N260Q-N267T-H272V-S279D-Y281P-K308Q-K309E-K312Q, Q3F-H15Y-S163P-S186R-Q215K-S225G-N227T-E232D-N252P-N260Q-N267T-H272V-S279D-Y281P-K308Q-K309E-K312Q, Q3F-H15Y-T17W-A49W-N59E-S163P-S186R-T218Q-S225G-N227T-G235W-S237W-N252P-H272P-K308Q-K309E-K312Q, Q3F-H15Y-T17W-A49W-N59E-S163P-S186R-D207N-T218Q-S225G-N227T-G235W-S237W-N252P-H272P-Y281P-K308Q-K309E-K312Q, Q3F-H15Y-T17W-A49W-N59E-S163P-S186R-T218Q-S225G-N227T-G235W-S237W-N252P-H272P-Y281P-K308Q-K309E-K312Q, Q3F-A49W-N59E-S163P-S186R-N227T-E232D-N252P-H272V-F276A-S279G-Y281P-K308E-K309E-K312E, Q3F-A49W-N59E-S163P-S186R-S225G-N227T-E232D-N252P-F276A-S279G-Y281P-K308E-K309E-K312E, Q3F-A49W-N59E-S163P-S186R-S225G-N227T-N252P-H272V-F276A-S279G-Y281P-K308E-K309E-K312E, Q3F-S163P-S186R-Q215K-S225G-N227T-E232D-N252P-N260Q-N267T-H272P-S279D-Y281P-K308Q-K309E-K312Q, Q3F-S163P-S186R-Q215K-N227T-E232D-N252P-N267T-H272V-F276A-S279G-Y281P-K308E-K309E-K312E, Q3F-S163P-S186R-Q215K-S225G-N227T-N252P-N267T-H272V-F276A-S279G-Y281P-K308E-K309E-K312E, Q3F-T17W-S163P-S186R-Q215K-S225G-N227T-E232D-G235W-N252P-N267T-H272V-F276A-S279G-Y281P-K308E-K309E-K312Q, Q3F-S163P-S186R-Q215K-S225G-N227T-E232D-N252P-N267T-H272V-F276A-S279G-Y281P-K308E-K309E-K312Q, Q3F-A49W-N59E-S163P-S186R-S225G-N227T-E232D-N252P-H272V-F276A-S279G-Y281P-K308E-K309E-K312E, Q3F-S163P-S186R-Q215K-S225G-N227T-E232D-N252P-N267T-H272V-F276A-S279G-Y281P-K308E-K309E-K312E, Q3F-H15Y-T17W-S163P-S186R-Q215K-S225G-N227T-E232D-G235W-N252P-N260Q-N267T-H272V-S279D-Y281P-K308Q-K309E-K312Q, Q3F-S163P-Q215K-S225G-N227T-E232D-N252P-N267T-H272V-F276A-S279G-Y281P-K308E-K309E-K312E, Q3F-H15Y-S163P-S186R-Q215K-S225G-N227T-E232D-G235W-N252P-N260Q-N267T-H272P-S279D-Y281P-K308Q-K309E-K312Q, Q3F-A49W-N59E-S163P-S186R-S225G-N227T-E232D-G235W-N252P-N260Q-H272P-F276A-S279D-Y281P-K308E-K309E-K312Q, Q3F-H15Y-T17W-S163P-S186R-Q215K-S225G-N227T-E232D-N252P-N260Q-N267T-H272V-S279D-Y281P-K308Q-K309E-K312Q, Q3F-H15Y-S163P-S186R-Q215K-S225G-N227T-E232D-N252P-N260Q-N267T-H272P-S279D-Y281P-K308Q-K309E-K312Q, Q3F-H15Y-S163P-S186R-Q215K-S225G-N227T-E232D-G235W-N252P-N267T-H272V-F276A-S279G-Y281P-K308Q-K309E-K312Q, Q3F-H15Y-T17W-S163P-S186R-Q215K-S225G-N227T-E232D-N252P-N267T-H272V-F276A-S279G-Y281P-K308Q-K309E-K312Q, Q3F-H15Y-A49W-N59E-V140I-S163P-S186R-Q215K-S225G-N227T-E232D-G235W-N252P-N260Q-N267T-H272V-S279D-Y281P-K308Q-K309E-K312Q, Q3I-H15Y-A49W-N59E-S163P-S186R-S225G-N227T-E232D-N252P-N260Q-N267T-H272V-S279D-Y281P-K308Q-K309E-K312Q, Q3I-H15Y-A49W-N59E-S163P-S186R-S225G-N227T-E232D-N252P-N260Q-N267T-H272P-S279D-Y281P-K308Q-K309E-K312Q, Q3I-H15Y-A49W-N59E-S163P-S186R-S225G-N227T-E232D-G235W-N252P-N260Q-H272V-S279D-Y281P-K308Q-K309E-K312E, Q3I-H15Y-A49W-N59E-S163P-S186R-S225G-N227T-E232D-G235W-N252P-N260Q-H272V-S279D-Y281P-K308E-K309E-K312Q, Q3I-H15Y-A49W-N59E-S163P-S186R-S225G-N227T-E232D-G235W-N252P-N260Q-H272V-S279D-Y281P-K308Q-K309E-K312Q, H15Y-A49W-N59E-S163P-S186R-S225G-N227T-E232D-G235W-N252P-N260Q-H272P-S279D-Y281P-K308Q-K309E-K312Q, H15Y-A49W-N59E-S163P-S186R-S225G-N227T-E232D-G235W-N252P-N260Q-H272V-S279D-Y281P-K308Q-K309E-K312Q, H15Y-A49W-N59E-S163P-S186R-S225G-N227T-E232D-N252P-N260Q-H272V-Y281P-K308Q-K309E-K312Q, H15Y-A49W-N59E-S163P-S186R-S225G-N227T-E232D-G235W-N252P-N260Q-H272V-S279D-Y281P-K309E-K312Q, H15Y-A49W-N59E-S163P-S186R-S225G-N227T-E232D-N252P-N260Q-H272P-S279D-Y281P-S288P-K308Q-K309E-K312Q, H15Y-A49W-N59E-S163P-S186R-S225G-N227T-E232D-N252P-N260Q-H272P-S279D-Y281P-K308Q-K309E-K312Q, H15Y-T17W-A49W-N59E-S163P-S186R-S225G-N227T-E232D-G235W-N252P-N260Q-H272V-S279D-Y281P-K308Q-K309E-K312Q, H15Y-T17W-A49W-N59E-S163P-S186R-S225G-N227T-E232D-N252P-N260Q-H272V-S279D-Y281P-K308Q-K309E-K312Q, A49W-N59E-S163P-S186R-S225G-N227T-E232D-N252P-H272V-F276A-S279G-Y281P-K308E-K309E-K312E and S163P-S186R-Q215K-S225G-N227T-E232D-N252P-N267T-H272V-F276A-S279G-Y281P-K308E-K309E-K312E, based on the numbering according to SEQ ID NO:1.

In very particularly preferred embodiments, a dispersin is used which exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% or 94%, and has at least 18 amino acid substitutions, wherein the dispersin has an amino acid substitution combination which is selected from the group consisting of Q3F-H15Y-V140I-S163P-S186R-Q215K-S225G-N227T-E232D-G235W-N252P-N260Q-N267T-H272V-S279D-Y281P-K308Q-K309E-K312Q, Q3F-H15Y-T17W-A49W-N59E-S163P-S186R-D207N-T218Q-S225G-N227T-G235W-S237W-N252P-H272P-Y281P-K308Q-K309E-K312Q, Q3F-H15Y-T17W-A49W-N59E-S163P-S186R-T218Q-S225G-N227T-G235W-S237W-N252P-H272P-Y281P-K308Q-K309E-K312Q, Q3F-T17W-S163P-S186R-Q215K-S225G-N227T-E232D-G235W-N252P-N267T-H272V-F276A-S279G-Y281P-K308E-K309E-K312Q, Q3F-H15Y-T17W-S163P-S186R-Q215K-S225G-N227T-E232D-G235W-N252P-N260Q-N267T-H272V-S279D-Y281P-K308Q-K309E-K312Q, Q3F-H15Y-S163P-S186R-Q215K-S225G-N227T-E232D-G235W-N252P-N260Q-N267T-H272P-S279D-Y281P-K308Q-K309E-K312Q, Q3F-A49W-N59E-S163P-S186R-S225G-N227T-E232D-G235W-N252P-N260Q-H272P-F276A-S279D-Y281P-K308E-K309E-K312Q, Q3F-H15Y-T17W-S163P-S186R-Q215K-S225G-N227T-E232D-N252P-N260Q-N267T-H272V-S279D-Y281P-K308Q-K309E-K312Q, Q3F-H15Y-S163P-S186R-Q215K-S225G-N227T-E232D-G235W-N252P-N267T-H272V-F276A-S279G-Y281P-K308Q-K309E-K312Q, Q3F-H15Y-T17W-S163P-S186R-Q215K-S225G-N227T-E232D-N252P-N267T-H272V-F276A-S279G-Y281P-K308Q-K309E-K312Q, Q3F-H15Y-A49W-N59E-V140I-S163P-S186R-Q215K-S225G-N227T-E232D-G235W-N252P-N260Q-N267T-H272V-S279D-Y281P-K308Q-K309E-K312Q, Q3I-H15Y-A49W-N59E-S163P-S186R-S225G-N227T-E232D-N252P-N260Q-N267T-H272V-S279D-Y281P-K308Q-K309E-K312Q, Q3I-H15Y-A49W-N59E-S163P-S186R-S225G-N227T-E232D-N252P-N260Q-N267T-H272P-S279D-Y281P-K308Q-K309E-K312Q, Q3I-H15Y-A49W-N59E-S163P-S186R-S225G-N227T-E232D-G235W-N252P-N260Q-H272V-S279D-Y281P-K308Q-K309E-K312E, Q3I-H15Y-A49W-N59E-S163P-S186R-S225G-N227T-E232D-G235W-N252P-N260Q-H272V-S279D-Y281P-K308E-K309E-K312Q, Q3I-H15Y-A49W-N59E-S163P-S186R-S225G-N227T-E232D-G235W-N252P-N260Q-H272V-S279D-Y281P-K308Q-K309E-K312Q and H15Y-T17W-A49W-N59E-S163P-S186R-S225G-N227T-E232D-G235W-N252P-N260Q-H272V-S279D-Y281P-K308Q-K309E-K312Q, based on the numbering according to SEQ ID NO:1.

The term “β-1,6-N-acetylglucosaminidase activity” as used herein is synonymous with “hexosaminidase activity” and/or “dispersin activity” and is determined as follows: To determine dispersin activity, 35 μl of dispersin assay solution (45 mM citrate buffer pH 5 with 0.5 mg/ml p-nitrophenyl-N-acetyl-β-D-glucosaminide) is added to a microtiter plate containing 5 μl of sample solution per well. Incubate the microtiter plate for 3 hours at room temperature. After incubation, 40 μl stop solution (0.4 M Na₂CO₃) was added and the absorption was measured at 405 nm. The activity values obtained are corrected by subtracting the absorption measurement obtained for a reference without dispersin to eliminate background noise. From this, the residual activity (RA) for the corresponding dispersin variant can be calculated in comparison to a reference dispersin.

The enzymes mentioned herein are preferably mature enzymes, i.e., the catalytically active molecule without signal and/or propeptide(s). Unless otherwise stated, the sequences indicated also refer to mature (processed) enzymes in each case.

In various embodiments of the invention, the corresponding enzyme is a freely available enzyme. This means that the enzyme can interact directly with all components of the agent and, if the agent is a liquid agent, that the enzyme is in direct contact with the solvent of the agent (e.g., water). In other embodiments, an agent may contain enzymes which form an interaction complex with other molecules or contain a “wrapping.” In this case, an individual enzyme molecule or a plurality of enzyme molecules may be separated from the other constituents of the agent by a surrounding structure. Such a separating structure can arise due to, but is not limited to, vesicles, such as a micelle or a liposome. However, the surrounding structure may also be a virus particle, a bacterial cell or a eukaryotic cell. In various embodiments, an agent can comprise cells of Bacillus pumilus or Bacillus subtilis that express the proteases according to the invention, or cell culture supernatants of such cells.

In the context of the present invention, the feature means that an enzyme has the indicated substitution(s) (or deletion or insertion), that it contains one (of the indicated) substitution(s) (or deletion or insertion) at the corresponding position, i.e., at least the indicated positions are not otherwise mutated or deleted, for example, by fragmentation of the enzyme. In various embodiments, the enzymes described herein have the sequence of the respective reference sequence except for the explicitly mentioned substitutions, i.e., are 100% identical to the respective reference sequence except for the substituted positions.

The identity of nucleic acid or amino acid sequences is determined by a sequence comparison. This sequence comparison is based on the BLAST algorithm established and commonly used in the prior art (cf. e.g., Altschul et al., “Basic local alignment search tool,” J. Mol. Biol., 1990,215: 403-410, and Altschul et al., “Gapped BLAST and PSI-BLAST: a new generation of protein database search programs,” Nucleic Acids Res., 1997, 25: 3389-3402) and occurs in principle by similar sequences of nucleotides or amino acids in the nucleic acid sequences or amino acid sequences being assigned to one another. A tabular assignment of the relevant positions is referred to as an alignment. A further algorithm available in the prior art is the FASTA algorithm. Sequence comparisons (alignments), in particular multiple sequence comparisons, are created using computer programs. The Clustal series (cf. e.g., Chenna et al., “Multiple sequence alignment with the Clustal series of programs,” Nucleic Acid Res., 2003, 31: 3497-3500), T-Coffee (cf. e.g., Notredame et al., “T-Coffee: A novel method for multiple sequence alignments,” J. Mol. Biol., 2000, 302: 205-217) or programs based on these programs or algorithms, for example, are frequently used. Also possible are sequence comparisons (alignments) using the computer program Vector NTI® Suite 10.3 (Invitrogen Corporation, 1600 Faraday Avenue, Carlsbad, California, USA) with the specified standard parameters, the AlignX module of which for the sequence comparisons is based on ClustalW, or Clone Manager 10 (use of the scoring matrix BLOSUM 62 for sequence alignment at amino acid level). Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid residues at the same positions or positions corresponding to one another in an alignment. In the case of amino acid sequences, the broader concept of homology takes conserved amino acid exchanges into account, i.e., amino acids having similar chemical activity, because these usually perform similar chemical activities within the protein. Therefore, the similarity of the compared sequences can also be indicated as percent homology or percent similarity. Identity and/or homology information can be provided regarding whole polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Often, such small regions perform essential functions for the overall activity of the protein. It may therefore be expedient to relate sequence matches only to individual, optionally small, regions. Unless otherwise stated, however, identity or homology information in the present application relates to the entire length of the particular nucleic acid or amino acid sequence indicated.

In the context of the present invention, the indication that an amino acid position corresponds to a numerically designated position in SEQ ID NO:1 therefore means that the corresponding position is associated with the numerically designated position in SEQ ID NO:1 in an alignment as defined above.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Several or alternative exchanges within the same polypeptide chain are separated by slashes. “130D/V” thus means that position 130 has mutated to D or V. In the case of insertions, additional amino acids are named according to the sequence position. In the case of deletions, the missing amino acid is replaced by a symbol, for example a star or a dash, or a Δ is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

It is possible for a person skilled in the art to use methods which are currently generally known, for example chemical synthesis or polymerase chain reaction (PCR), in conjunction with molecular biology and/or protein-chemical standard methods, to produce the corresponding nucleic acids and even complete genes on the basis of known DNA and/or amino acid sequences. Such methods are known, for example, from Sambrook, J., Fritsch, E. F. and Maniatis, T. 2001. Molecular cloning: a laboratory manual, 3. Edition Cold Spring Laboratory Press.

The term “fragrance” as used herein is synonymous with the terms “perfume”, “perfume composition”, “fragrance composition”, “perfume substance”, “scent”, “scented oil”, “odorous substance”, “odorant”, and the like and means a substance that stimulates the sense of smell.

Suitable perfumes and perfume constituents can be found in the CTFA (Cosmetic, Toiletry and Fragrance Association) 1992 International Buyers Guide, published by CFTA Publications, and in the OPD 1993 Chemicals Buyers Directory 80th Annual Edition, published by Schnell Publishing Co.

There are no restrictions on the preferred fragrances that can be used. In particular, synthetic or natural fragrance compounds of the types esters, ethers, aldehydes (fragrance aldehydes), ketones (fragrance ketones), alcohols, hydrocarbons, acids, carboxylic acid esters, aromatic hydrocarbons, aliphatic hydrocarbons, saturated and/or unsaturated hydrocarbons and mixtures thereof can be used as fragrances.

Both individual fragrance compounds, e.g., synthetic products of esters, ethers, aldehydes, ketones, alcohols and hydrocarbons, as well as mixtures thereof can be used. Preferably, however, mixtures of different fragrances are used which together produce an appealing scent. Such mixtures may also contain natural aromatic substance mixtures, such as those available from plant sources, e.g., pine, citrus, jasmine, patchouli, rose or ylang-ylang oils. If it is to be perceptible, a perfume has to be volatile, with the molecular weight, in addition to the type of functional groups and the structure of the chemical compound, also playing an important role. Most perfumes have a molecular weight of up to approximately 200 g/mol, while molecular weights of 300 g/mol and more are the exception. Due to the varying volatility of fragrances, the smell of a perfume consisting of several fragrances changes during evaporation, with the olfactory impressions being divided into “top note”, “middle note” or “body” and “end note” or “dry down”. Since the perception of a smell also depends significantly on the intensity of the smell, the top note of a perfume or scent consists not only of slightly volatile compounds, while the end note usually consists of less volatile, i.e., persistent fragrances.

Suitable fragrances of the ester type are, e.g., benzyl acetate, phenoxyethyl isobutyrate, p-tert-butylcyclohexyl acetate, linalyl acetate, dimethylbenzylcarbinyl acetate (DMBCA), phenylethyl acetate, ethylmethylphenyl glycinate, allylcyclohexyl propionate, styrallyl propionate, benzyl salicylate, cyclohexyl salicylate, floramate, melusate, and jasmacyclate.

Hydrocarbon-type odorous compounds include, for example, terpenes such as limonene and pinene.

Suitable ether-type fragrances include, for example, benzyl ethyl ether and ambroxan.

Suitable alcohol-type fragrances include, for example, 10-undecen-1-ol, 2,6-dimethylheptan-2-ol, 2-methylbutanol, 2-methylpentanol, 2-phenoxyethanol, 2-phenylpropanol, 2-tert-butylcyclohexanol, 3,5,5-trimethylcyclohexanol, 3-hexanol, 3-methyl-5-phenylpentanol, 3-octanol, 1-octen-3-ol, 3-phenylpropanol, 4-heptenol, 4-isopropylcyclohexanol, 4-tert-butylcyclohexanol, 6,8-dimethyl-2-nonanol, 6-nonen-1-ol, 9-decen-1-ol, a-methyl benzyl alcohol, a-terpineol, amyl salicylate, benzyl alcohol, benzyl salicylate, β-terpineol, butyl salicylate, citronellol, cyclohexyl salicylate, decanol, dihydromyrcenol, dimethyl benzyl carbinol, dimethyl heptanol, dimethyl octanol, ethyl salicylate, ethylvanillin, anethol, eugenol, geraniol, heptanol, hexyl salicylate, isoborneol, isoeugenol, isopulegol, linalool, menthol, myrtenol, n-hexanol, nerol, nonanol, octanol, para-menthan-7-ol, phenylethyl alcohol, phenol, phenyl salicylate, tetrahydrogeraniol, tetrahydrolinalool, thymol, trans-2-cis-6-nonadienol, trans-2-nonen-1-ol, trans-2-octenol, undecanol, vanillin, cinnamyl alcohol, wherein, if a plurality of fragrance alcohols are provided, these can be selected independently of one another.

Suitable ketone-type fragrances can be any ketone that can impart a desired scent or feeling of freshness. Mixtures of various ketones can also be used. The ketone can be selected, for example, from the group consisting of Buccoxime, isojasmone, methyl-β-naphthyl ketone, musk indanone, tonalide/musk plus, α-damascone, β-damascone, δ-damascone, iso-damascone, damascenon, damask rose, methyl dihydrojasmonate, menthone, carvone, camphor, fenchone, α-ionene, β-ionone, dihydro-β-ionone, γ-methylionone, fleuramone, dihydrojasmone, cis-jasmone, iso-E-super, methylcedrenyl ketone or methylcedrylone, acetophenone, methylacetophenone, para-methoxyacetophenone, benzylacetone, benzophenone, para-hydroxyphenylbutanone, celery ketone or livescone, 6-isopropyldecahydro-2-naphtone, dimethyloctenone, frescomenthe, 4-(1-ethoxyvinyl)-3,3,5,5-tetramethylcyclohexanone, methylheptenone, 2-(2-(4-methyl-3-cyclohexen-1-yl)-propyl)-cyclopentanone, 1-(para-menthen-6(2)-yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl or kassion, gelsone, Hexalone, Isocyclemone E, methylcyclocitron, methyl lavender ketone, Orivone, para-tert-butylcyclohexanone, verdone, Delphone, muscone, Neobutenone, plicatone, Veloutone, 2,4,4,7-tetramethyl-oct-6-en-3-one, tetrameran, hedione and mixtures thereof. The ketone can preferably be from α-damascone, δ-damascone, iso-damascone, carvone, γ-methylionone, iso-E-super, 2,4,4,7-tetramethyl-oct-6-en-3-one, benzylacetone, β-damascone, damascenon, methyldihydrojasmonate, methylcedrylone, hedione and mixtures thereof.

Suitable aldehyde-type fragrances can be any aldehyde that, like ketone-type fragrances, produces a desired scent or feeling of freshness. These can also be individual aldehydes or mixtures of aldehydes. Suitable aldehydes are, for example, melonal, triplal, ligustral, adoxal, anisaldehyde, cymene, ethylvanillin, Florhydral, helional, heliotropin, hydroxycitronellal, Koavone, laurinaldehyde, Lyral, methylnonylacetaldehyde, para-tert-bucinal, phenylacetaldehyde, undecylenic aldehyde, vanillin, 2,6,10-trimethyl-9-undecenal, 3-dodecen-1-al, α-n-amylcinnamaldehyde, 4-methoxybenzaldehyde, benzaldehyde, 3-(4-tert-butylphenyl)-propanal, 2-methyl-3-(paramethoxyphenyl propanal), 2-methyl-4-(2,6,6-trimethyl-2(1)-cyclohexene-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl) propanal, decylaldehyde, 2,6-dimethyl-5-heptenal, 4-(tricyclo[5.2.10-(2,6)]-decylidene-8)-butanal, octahydro-4,7-methano-1H-indene carboxaldehyde, 3-ethoxy-4-hydroxybenzaldehyde, para-ethyl-α-dimethyl-hydrocinnamaldehyde, α-methyl-3,4-(methylenedioxy)-hydrocinnamaldehyde, 3,4-(methylenedioxy)benzaldehyde, α-n-hexylcinnamaldehyde, m-cymene-7-carboxaldehyde, α-methyl phenyl acetaldehyde, 7-hydroxy-3,7-dimethyloctanal, undecenal, 2,4,6-trimethyl-3-cyclohexene-1-carboxaldehyde, 4-(3)-(4-methyl-3-pentenyl)-3-cyclohexene carboxaldehyde, 1-dodecanal, 2,4-dimethyl-cyclohexene-3-carboxaldehyde, 4-(4-hydroxy-4-methylpentyl)-3-cylohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanoindan-1- or -2-carboxaldehyde, 3,7-dimethyloctan-1-al, 1-undecanal, 10-undecen-1-al, 4-hydroxy-3-methoxybenzaldehyde, 1-methyl-3-(4-methylpentyl)-3-cyclohexene carboxyaldehyde, trans-4-decenal, 2,6-nonadienal, para-tolyl acetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,1-dimethyl-3-oxa-5,9-undecadien-1-al), hexahydro-4,7-methanoindan-1-carboxaldehyde, 2-methyloctanal, α-methyl-4-(1-methylethyl)benzeneacetaldehyde, 6,6-dimethyl-2-norpinene-2-propionaldehyde, para-methyl phenoxy acetaldehyde, 2-methyl-3-phenyl-2-propen-1-al, 3,5,5-trimethylhexanal, hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal, 1-para-menthen-q-carboxaldehyde, citral, liliencitral, 1-decanal, 2,4-dimethyl-3-cyclohexen-1-carboxaldehyde or mixtures thereof. Preferred aldehydes can be selected from cis/trans-3,7-dimethyl-2,6-octadien-1-al, heliotropin, 2,4,6-trimethyl-3-cyclohexene-1-carboxaldehyde, 2,6-nonadienal, α-n-amylcinnamaldehyde, α-n-hexylcinnamaldehyde, para-tert-bucinal, Lyral, cymene, methylnonylacetaldehyde, trans-2-nonenal, Lilial, trans-2-nonenal and mixtures thereof.

The fragrances may also be natural aromatic substance mixtures, such as those available from plant sources, e.g., pine, citrus, jasmine, patchouli, rose or ylang-ylang oils. Also suitable are nutmeg, sage, chamomile, clove, mint, cinnamon leaf, lime blossom, juniper berry, vetiver, frankincense, galbanum and labdanum oil as well as orange blossom, neroli, orange peel and sandalwood oil. The fragrances can also be essential oils, e.g., angelica root, anise, arnica blossom, basal, bay, champaca blossom, silver fir, silver fir cone, elemi, eucalyptus, fennel, pine needle, geranium, ginger grass, guaiac wood, gurjun balsam, helichrysum, ho leaf, ginger, iris, cajeput, calmus, camphor, canaga, cardamom, cassia, copaiva balsam, coriander, mint, caraway, cumene, lavender, lemongrass, lime, mandarin, melissa, musk seed, myrrh, niaouli, oregano, palmarosa, balsam Peru, petitgrain, pepper, peppermint, allspice, rosemary, celery, spike, star anise, turpentine, thuja, thyme, verbena, wormwood, wintergreen, hyssop, cinnamon, citronella, lemon and cypress oil.

Preferably, the fragrance comprises at least one note (compound) selected from: α-isomethyl ionone, benzyl salicylate, citronellol, coumarin, hexylcinnamal, linalool, pentanoic acid, 2-methyl, ethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-iron, β-phenylethylbenzoate, α-santalol, cedrol, cedryl acetate, cedryl formate, cyclohexyl salicyate, γ-dodecalactone and β-phenylethylphenylacetate.

Useful fragrance constituents include substances of both natural and synthetic origin. They include individual compounds and mixtures. Specific examples of such constituents can be found in Fenaroli's Handbook of Flavor Ingredients, 1975, CRC Press; Synthetic Food Adjuncts, 1947 by MB Jacobs, edited by Van Nostrand; or Perfume and Flavor Chemicals by S. Arctander 1969, Montclair, N.J. (USA).

Aromatic substance mixtures preferably include top notes as defined by Poucher (Journal of the Society of Cosmetic Chemists 6(2):80, 1955). Preferred top notes are selected from citrus oils, linalool, linalyl acetate, lavender, dihydromyrcenol, rose oxide and cis-3-hexanol. The International Fragrance Association publishes lists of fragrance ingredients (http://www.ifraorg.org/en-us/ingredients#.XMqixOgza71). The list of 3999 substances reported as used in fragrances in 2015 is hereby incorporated by reference (https://ifrafragrance.org/initiatives/transparency/ifra-transparency-list).

It is well known that the degree of hydrophobicity of a fragrance can be correlated with its octanol/water partition coefficient P. The octanol/water partition coefficient of a fragrance is the ratio between its equilibrium concentration in octanol and in water. A fragrance with a higher partition coefficient P is more hydrophobic. Conversely, a fragrance with a smaller partition coefficient P is more hydrophilic. Since the partition coefficients of fragrances are generally high, they are conveniently expressed in the form of their logarithm to base 10, log P. The log P value is a constant defined as follows:

$$log P = {log}_{10} \frac{c ( octanol )}{c ( water )}$$

In preferred embodiments, the log P values are calculated using the program “C LOG P” available from Daylight Chemical Information Systems. The “calculated log P” (C log P) is determined by the fragment approach of Hansch & Leo (cf. Leo, in Comprehensive Medicinal Chemistry, Vol. 4, Hansch, et al., Eds., p. 295, Pergamon Press, 1990). In one embodiment, the C log P value of the fragrance to be used in washing and/or cleaning agents according to the invention is in the range from approximately −1.0 to approximately 4.0. In further embodiments, the C log P of the fragrance ranges from approximately 0.0 to approximately 4.0, approximately 1.0 to approximately 4.0, approximately 2.0 to approximately 4.0, or approximately 3.0 to approximately 4.0. In still further embodiments, the C log P of the fragrance ranges from approximately 0.0 to approximately 3.0, approximately 1.0 to approximately 3.0, or approximately 2.0 to approximately 3.0.

Non-limiting exemplary fragrances that have C log P values in the range of approximately −1.0 to approximately 4.0 are, for example, allyl heptanoate, anethole USP, benzaldehyde, benzyl acetate, cis-3-hexenyl acetate, cis-jasmone, coumarin, dihydromyrcenol, dimethylbenzylcarbinyl acetate, ethylvanillin, eucalyptol, eugenol, isoeugenol, isobutyl salicylate, floracetate, geraniol, hydroxycitronellal, Koavone, liffarom, dihydrolinalool, linalool, methyl anthranilate, methyl-β-naphthyl ketone, methyl dihydrojasmonate, nerol, nonalactone, orange blossom ether, phenylethyacetate, phenylethyl alcohol, phenylpropyl alcohol, phenoxyethyl isobutyrate, phenoxanol, α-terpineol, tetrahydrolinalool, β-terpineol, vanillin and mixtures thereof.

It is advantageous to encapsulate fragrances that have a low C log P value (i.e., those that have a greater tendency to disperse in water), preferably with a C log P value of less than approximately 3.0. These substances with a relatively low boiling point and relatively low C log P are called “delayed blooming” perfume constituents and include one or more of the following substances: allyl caproate, amyl acetate, amyl propionate, anisaldehyde, anisole, benzaldehyde, benzyl acetate, benzyl acetone, benzyl alcohol, benzyl formate, benzyl isovalerate, benzyl propionate, β-γ-hexenol, camphor gum, laevo-carvone, d-carvone, cinnamic alcohol, cinamyl formate, cis-jasmone, cis-3-hexenyl acetate, cumin alcohol, cyclal C, dimethylbenzylcarbinol, dimethylbenzylcarbinol acetate, ethyl acetate, ethyl acetoacetate, ethyl amyl ketone, ethyl benzoate, ethyl butyrate, ethylhexyl ketone, ethyl phenyl acetate, eucalyptol, eugenol, fenchyl acetate, floracetate (tricyclodecenyl acetate), fruten (tricyclic codecenyl propionate), geraniol, hexenol, hexenyl acetate, hexyl acetate, hexyl formate, hydrotropic alcohol, hydroxycitronellal, indone, isoamyl alcohol, isomenthone, isopulegyl acetate, isoquinolone, ligustral, linalool, linalool oxide, linalyl formate, menthone, menthylacetophenone, methyl amyl ketone, methyl anthranilate, methyl benzoate, methyl benzyl acetate, methyl eugenol, methyl heptenone, methyl heptine carbonate, methyl heptyl ketone, methyl hexyl ketone, methyl phenyl carbinyl acetate, methyl salicylate, methyl n-methyl anthranilate, nerol, octalactone, octyl alcohol, p-cresol, p-cresol methyl ether, p-methoxyacetophenone, p-methylacetophenone, phenoxyethanol, phenylacetaldehyde, phenylethyl acetate, phenylethyl alcohol, phenylethyldimethylcarbinol, prenyl acetate, propylbornate, pulegone, rose oxide, safrole, 4-terpinenol, α-terpinenol and/or viridin. Another group of fragrances that can be used with the present invention are aromatherapy substances, which can contain many components also used in perfumery, including components of essential oils such as clary sage, eucalyptus, geranium, lavender, mace extract, neroli, nutmeg, mint, sweet violet leaf and valerian.

In one embodiment, the fragrance to be incorporated into the agent according to the invention has a boiling point of less than approximately 250° C. and a calculated water-octanol partition coefficient (C log P) of less than approximately 3, known as Quadrant I fragrance components.

In one embodiment, the fragrance to be incorporated into the agent according to the invention has a boiling point of more than approximately 250° C. and a calculated water-octanol partition coefficient (C log P) of less than approximately 3, known as Quadrant II fragrance components.

In one embodiment, the fragrance to be incorporated into the agent according to the invention has a boiling point of less than approximately 250° C. and a calculated water-octanol partition coefficient (C log P) of more than approximately 3, known as Quadrant III fragrance components.

In one embodiment, the fragrance to be incorporated into the agent according to the invention has a boiling point of more than approximately 250° C. and a calculated water-octanol partition coefficient (C log P) of more than approximately 3, known as Quadrant IV fragrance components.

Suitable fragrance raw materials include, but are not limited to, ethyl-2,4-decadienoate, allyl heptoate, amyl acetate, ethyl butyrate, grapefruit peel (C and A), prenyl acetate, pinoacetaldehyde, 2,6-nonadienol, 3,6-nonadienol, cis-6-nonenol, excital, ebanol, polysantol, orange juice carbonyls, lemon juice carbonyls, orange sinensal, paradiff, tangerinal, benzaldehyde, mandarin aldehyde, undecalactone, norlimbanol, decylaldehyde, trans-2-hexenal, trans-2-decenal, damascenon, 2-isobutylthiazole, 4-methyl-4-mercaptopentan-2-one, Corps Cassis 0.1% TEC, patchouli, 2-methoxy-4-vinylphenol, pyridine acetyl 10%, sulfurol, diacetyl, furaneol, maple lactone, allylamyl glycolate, ambroxan, α-damascon, α-damascene, cetalox, cyclal c, cedramber, cyclogalbanate, galbex, cymene, nerol, Florhydral, para-tert-bucinal, isocyclocitral, fructon, methylisobutenyltetrahydropyran, fruten, Delphone, ethylmethylphenylglycidate, violiff, δ-damascone, δ-damascene, ambrox, calon, isoeugenol, hivernal, methylo-napthyl ketone, ozonil, benzyl salicylate, spirogalbon, cinnamon alcohol, javanol, dihydroisojasmonate, adoxal, kharismal, pyrazine, ethyl anthranilate, aldehyde supra, bacdanol, anethole, irisanthem, yara yara, keon, cis-3-hexenyl salicylate, methyl nonyl ketone, coumarin, γ-dodecalactone, applinate, eucalyptol, intreleven aldehyde, heliotropin, indole, manzanate, trans-4-decenal, oxane, neobutanone, clonal, methyloctyne carbonate, floralozone, methylheptine carbonate, methylnonylacetaldehyde, cashmeran, phenoxyethyl isobutyrate, phenylacetaldehyde, undecylaldehyde, aurantiol, nectaryl, Buccoxime, laurie aldehyde, nirvanol, trifernal, pyrazobutyl, Veloutone, anisaldehyde, para-menthene, isovaleraldehyde 0.1% DPG, liminal, labienoxime, rhubofix, isopropylquinoline, 4-(2,6,6-trimethyl-1-cyclohexenyl)-3-butenone-2,(3aR-(3a-α,5a-β,9a-α,9b-β))-dodecahydro-3a,6,6,9a-tetramethylnaphtha-(2,1-b)-furan, 2,6-dimethyl-5-heptenal, 3,7-dimethyl-1,6-octadien-3-ol, 3-methyl-2-buten-1-yl acetate, 3,7-dimethyl-2,6-octadienonitrile, 2,4-dimethylcyclohexene-3-carbaldehyde, ethylmethyldioxolane acetate, 4-(2,6,6-trimethyl-1,3-cyclohexadienyl)-3-buten-4-one, cis-3-hexenyl acetate, lauric acid, tricyclodecenyl acetate, para-cresyl methyl ether, 7-acetyl-1,2,3,4,5,6,7,8-octahydro-1,1,6,7-tetramethylnaphthalene, 3-buten-2-one, 3-methyl-4-(2,6,6-trimethyl-2-cyclohexen-1-yl), (cyclohexyloxy)-acetic acid, 2-propenyl ester, 4-(2,6,6-trimethyl-2-cyclohexen-1-yl), methyl-3,4-dioxy-(cylcoacetonyl)-benzene, 2,6-dimethyl-2,6-octadien-8-ol, ortho-tert-butylcyclohexanyl acetate, caproic acid, methoxybenzaldehyde, 3-(3-isopropylphenyl)-butanal, iso-2-methoxy-4-(2-propenyl)-phenol, 1-methyl-4-isopropenyl-1-cyclohexene, methylphenylcarbonyl acetate, hexahydro-4,7-methano-1H-indene-5-(or 6)-yl propionate, 3,7-dimethyl-2,6-octadienal, 3,3-dimethyl-5-(2,2,3-trimethyl-3-cycloenten-1-yl)-4-penten-2-ol, 2-methoxy-4-(2-propenyl)-phenol, 3,7-dimethyl-6-octen-1-ol, allyl heptanoate, 1,3-oxathian, (all-E)-α-sinensal, 2,6,10-trimethyl-2(E),6(E),9(E),11-dodecatetraenal, p-1-menthene-8-thiol, 4-methyl-3-decen-5-ol, ethyl caproate, 4-penten-1-one, 1-(5,5-dimethyl-1-cyclohexen-1-yl), 1H-indene-a-propanal, 2,3-dihydro-1,1-dimethyl-(9CI), 4-dodecenal, 3-cyclohexene-1-carboxaldehyde, 2,4-dimethyl, 2,6-nonenol, 3-p-cumenylpropionaldehyde-4-(1-methylethyl)-benzenepropanal, 1-(2,6,6-trimethyl-1,3-cyclohexanedienyl)-2-buten-1-one, 6-(Z,3-pentenyl)-tetrahydro-(2H)-pyranon-2,3-methyl-(cis-2-penten-1-yl)-2-cyclopenten-1-one, 6,6-dimethylbicyclo-(3.1.1)-hept-2-en-2-proponal, 4-penten-2-ol, undeclactone, 4-methoxybenzaldehyde, 3-dodecenal, animal fragrances such as musk oil, civet, castoreum, ambergris, plant fragrances such as nutmeg, cardamom, ginger, cinnamon, orange blossom, ylang, tuberose, mimosa, narcissus, carrot seed, jasmine, olibanum and rose extract, patchouli, geranium, orange, mandarin, sandalwood, bergamot, rosemary, mint, peppermint, lemon, lavender, citronella, chamomile, clove, sage, neroli, labdanum, eucalyptus and verbena oil, cedarwood, vetiver, lavender, acetophenone, C₁₂-aldehydes, C₁₄-aldehydes, C₁₈-aldehydes, allyl caprylate, dimethylindane derivatives, anisaldehyde, benzyl acetate, benzyl alcohol and ester derivatives, benzyl propionate, β-γ-hexanol, borneol, butyl acetate, camphor, carbitol, carvone, cinnamaldehyde, cinnamyl acetate, cinnamyl alcohol, cis-3-hexanol and ester derivatives, cis-3-hexenylmethyl carbonate, cis-jasmone, citral, citronnellol and ester derivatives, cuminaldehyde, cyclamenaldehyde, damascone, decanol, estragole, δ-muscenone, dihydromyrcenol, dimethylbenzylcarbinol, 6,8-dimethyl-2-nonanol, dimethylbenzylcarbinyl butyrate, ethyl isobutyrate, ethyl propionate, ethyl caprylate, ethyl cinnamate, ethyl hexanoate, ethyl valerate, exaltolide, fenchone, galaxolide, geraniol and ester derivatives, hedione, helional, 2-heptonone, hexenol, hexyl salicylate, hydroxycitronellal, isoamyl isovalerate, iso E super, linalool acetate, Lilial, Lyral, majantol, mayol, menthol, p-methylacetophenone, methylcedrylone, methyl dihydrojasmonate, methyleugenol, Mugetanol, para-hydroxyphenylbutanone, phenoxynol, phenylacetaldehyde dimethyl acetate, phenoxyethyl isobutyrate, phenylethyl alcohol, pinene, sandalor, sanjinol, santalol, thymol, terpene, tonalide, 3,3,5-trimethylcyclohexanol, undecylenaldehyde, linalool, citronellol, isobornyl acetate, para-tert-butylcyclohexyl acetate, linalyl acetate, dihydro-nor-dicyclopentadienyl acetate, dihydro-nor-dicyclopentadienyl propionate, amyl salicylate, para-isopropylα-octylhydrocinnamaldehyde, hexylcinnamaldehyde, hydroxycitronellal, γ-methylionone, methyl-β-naphthyl ketone, γ-undecalactone, eugenol, musk xylene, 1,3,4,6,7,8-hexahydro-4,6,6,7,8,8-hexamethylcyclopenta-γ-2-benzopyran, 4-acetyl-6-tertiarybutyl-1,1-dimethylindane, 6-acetyl-1,1,3,4,4,6-hexamethyltetrahydronaphthalene, β-naphthyl ethyl ether, methyl cedrenyl ketone, geranyl nitrile, α-Ionone, α-β-ionone, benzyl isoeugenol, amyl cinnamaldehyde, β-γ-hexenol, orange CP, 2-methyl-3-(para-iso-propylphenyl)-propionaldehyde, trichloromethylphenylcarbinyl acetate, nonanediol 1,3-acetate, citrathal, tetrahydromuguol, ethylene brassylate, musk ketone, musk tibetine, phenylethyl acetate, oakmoss 25%, stemone, citronellyl nitrile, ethyl linalool, undecavertol, methylphenylcarbinyl acetate, 6-nonen-1-ol, (6Z)-cis-3-hexen-1-ol (β-γ-hexenol), ambrox DL, ozonepropanal (floralozone), 3-methyl-5-(2,2,3-trimethyl-3-cyclopenten-1-yl)-pent-4-en-2-ol (ebanol), phenethyl isobutyrate, bourgeonal, ethyl-2-methyl-1,3-dioxolane-2-acetate (fructon), bigaradoxide, allylcyclohexylpropionate, tetrahydrolinalool, trimofix O, neofolion, citronellyloxyacetaldehyde, romanolide, β-pinene, karanal, vertenex, o-tert-butylcyclohexyl acetate (verdox), γ-decalactone, oxalone (calone 1951), cinnamaldehyde, dihydro-β-ionone, ethyl acetate, cyclemax, d-limonene, vivaldie, trans-anethole, cis-3-hexenylbutyrate, floracetate, trans-pinoacetaldehyde, dodecanal, rose oxide, undecanal (undecylaldehyde), allyl caproate, romascone, a-iron, hexyl acetate, liffarome, vertoliff, nonanal, methyl 2-octynoate, β-ionone, ethyloenanthate, maltol, α-damascone, methyl 2-nonynoate, γ-nonalactone, dimetol, methyl pamplemousse, methyl ionone (xandralia), 2-nonen-1-al, (E)-2,(Z)-6-nonadienal, ethyl-2-methylbutyrate, melonal, isoamyl acetate, 2,6-nonadien-1-ol, (2E,6Z)-(nonadienol), isobutylquinoline, 2-methylundecanal, methyl anthranilate, cis-3-nonadienol, cis-6-nonadienol, ethylvanillin, Neobutenone, triplal or ligustral, 10-undecenal, citronellal, N-decanal, vanillin, L-carvone, octanal, methyl benzoate and mixtures thereof.

In one embodiment, the agent according to the invention contains a plurality of the above-mentioned fragrances.

Washing and/or cleaning agents according to the invention contain approximately 0.01 to 5 wt. %, preferably 0.05 to 3 wt. %, particularly preferably 0.1 to 1 wt. % of fragrances, based on the total weight of the agent.

A washing and/or cleaning agent composition usually contains several fragrances and/or fragrance constituents. In the compositions of the present invention, 5 or more, preferably 10 or more, preferably 20 or more, more preferably 40 or more different fragrances or fragrance constituents are present. In one embodiment of the invention, 50 or more, for example 55 or more, 60 or more, 65 or more, 70 or more, 75 or more, 80 or more, 85 or more, 90 or more, 95 or more, 100 or more, different fragrances or fragrance constituents may also be present.

In preferred embodiments, the fragrances are pre-formulated in so-called fragrance preparations. In preferred embodiments, the fragrance preparations comprise at least one and increasingly preferably at least 2, 3, 4, 5, 6, 7, 8, 9, 10, 11, 12, 13, 14, 15, 16, 17, 18, 19, 20 or more fragrances, and at least one solvent, such as dipropylene glycol or isopropylene myristate. Agents according to the invention may also contain one or more, e.g., 2, 3, 4, 5, 6, 7, 8, 9, 10 or more, such pre-formulated fragrance preparations.

In particularly preferred embodiments, the at least one and increasingly preferably the at least two, three, four, five, six, seven or more fragrance(s) is/are selected from the group consisting of α-isomethyl ionone, benzyl salicylate, citronellol, coumarin, hexyl cinnamal, linalool, pentanoic acid, 2-methylethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-iron, β-phenylethylbenzoate, α-santalol, cedrol, cedryl acetate, cedryl formate, cyclohexyl salicyate, γ-dodecalactone and β-phenylethylphenylacetate and mixtures thereof.

In preferred embodiments, the at least one fragrance is selected from the group consisting of 2-phenylethanol, hexanal, bornan-2-one, 3,7-dimethyl-3-octanol, linalool, allyl ionone, 3-methyl-4-(2,6,6-trimethyl-1-cyclohexen-1-yl)-3-buten-2-one, camphene, p-menth-1-en-8-yl acetate, pin-2(3)-ene, methyl N-methylanthranilate, isobutyl salicylate, isopentyl salicylate, β-caryophyllene, 2-tert-butylcyclohexyl acetate, (1S-cis)-1,2,3,4,5,6,7,8-octahydro-7-isopropylidene-1,4-dimethylazulene, coumarin, methyl 2-naphthyl ether, 2′-acetonapthone, ethyl 2-naphthyl ether, methyl benzoate, 1-phenylethyl acetate, eugenol, 4-(1,1-dimethylethyl)cyclohexanol, p-menth-1-en-8-ol, acetophenone, d-p-mentha-1(6),8-dien-2-one, p-mentha-1,4-diene, benzyl alcohol, benzaldehyde, (2,2-dimethoxyethyl)benzene, diphenyl ether, α-hexylcinnamaldehyde, 3-phenyl-2-propenoate, benzyl cinnamate, 2-phenylethyl acetate, phenoxyethyl isobutyrate, 3-p-cumenyl-2-methylpropionaldehyde, anethole, 5-heptyldihydro-2(3H)-furanone, 4-methylanisole, 4-methoxybenzyl alcohol, ethyl butyrate, 1,4-dioxacycloheptadecan-5,17-dione, oxacyclohexadecan-2-one, (E)-3,7-dimethyl-2,6-octadien-1-ol acetate, 3,7-dimethyloctan-1-ol, citronellol, geraniol, nerol, 2,6-dimethyl-5-heptenal, 7-hydroxycitronellal, oxacycloheptadecan-2-one, myristic acid isopropyl ester, 2-methylundecanal, methyloct-2-ynoate, hexan-1-ol, diethanolamine, methylnon-2-enoate, 2-undecanone, decan-1-ol, decanal, undecanal, undec-10-enal, dodecanal, 3,7-dimethyl-1,6-octadien-3-ol acetate, phenyl salicylate, benzyl salicylate, 3-hydroxy-2-methyl-4H-pyran-4-one, methyl salicylate, benzyl benzoate, 1,3-benzodioxole-5-carboxaldehyde, indole, 3-ethoxy-4-hydroxybenzaldehyde, 4-hydroxy-3-methoxy-benzaldehyde, 4′-methylacetophenone, 4-isopropylbenzaldehyde, 2-benzylidenheptanal, 2-phenoxyethanol, anisaldehyde, 7-methyl-3-methylene octa-1,6-diene, allyl hexanoate, isopentyl acetate, octanal, C9 aldehyde, exo-1,7,7-trimethylbicyclo-[2.2.1]-hept-2-yl acetate, 4-(2,6,6-trimethylcyclohex-2-en-1-yl)-but-3-en-2-one, 1-(2,6,6-trimethyl-1-cyclohexen-1-yl)pent-1-en-3-one, 3-methyl-4-(2,6,6-trimethyl-2-cyclohexen-1-yl)-3-buten-2-one, pin-2(10)-ene, 2,6-di-tert-butyl-p-cresol, methyl anthranilate, 2-heptylcyclopentanone, dipentene (crude), phenylmethylesteracetic acid, 4-allylanisole, (2Z)-3,7-dimethyl-2,6-octadien-1-ol-1-acetate, 2,6,10-trimethyl-9-undecenal, 2-aminoethanol, allyl heptanoate, hexyl acetate, 9-undecenal, citronellyl acetate, α-, α-dimethylphenethyl acetate, cineole, 2,6,6-trimethylbicyclo-[3.1.1]-heptane, longifolene, cyclopentadecanone, DL-borneol, β-patchoulene, p-mentha-1(7),2-diene, (2E,6Z)-nona-2,6-dien-1-al, p-mentha-1,4(8)-diene, γ-decalactone, cis-3-hexenol, (1R,4R,6R,10S)-4,12,12-trimethyl-9-methylene-5-oxatricyclo-[8.2.0.04,6]-dodecane, 3-methyl-2-buten-1-ol acetate, methyl ionone, pentyl salicylate, butyl salicylate, dodecanonitrile, 4,7-dimethyl-6-octen-3-one, 4-phenylbutan-2-one, acetaldehydeethylphenylethyl acetal, 2-methoxy-4-propylphenol, ethyl 2-ethylhexanoate, (Z)-3,7-dimethylocta-1,3,6-triene, α-butylbenzeneacetonitrile, phenylethyl methyl ether, (Z)-3-hexen-1-ol acetate, octahydro-2H-1-benzopyran-2-one, farnesol, 5-methyl-5-phenylhexan-3-one, 2-ethyl-3-hydroxy-4-pyrone, 2,4,6-trimethyl-4-phenyl-1,3-dioxane, citral, 3-(p-methoxyphenyl)-2-methylpropionaldehyde, 4-(4-hydroxyphenyl)butan-2-one, patchouli alcohol, tetrahydrocitral, (R)-p-mentha-1,8-diene, hexyl salicylate, 1-p-mentha-1(6),8-dien-2-one, trans-hex-2-enal, β-4-dimethyl-3-cyclohexene-1-propanal, [3aR-(3aa,5ab,9aa,9bb)]-dodecahydro-3a,6,6,9α-tetramethylnaphtho-[2,1-b]-furan, ethyl 2-methylbutyrate, [2-(1-propoxyethoxy)ethyl]benzene, phosphoric acid, 1-(2,6,6-trimethyl-2-cyclohexen-1-yl)pent-1-en-3-one, oxacycloheptadec-7-en-2-one, terpineol, pine oil, mandarin oil, orange oil (sweet), peppermint oil, ionone, patchouli oil, parsley oil, vetiver oil, lavender oil, resin acids & rosin acids (hydrogenated, Me-esters), benzoin resin, α,α-dimethylphenethylbutyrate, 3,7-dimethyl-1,6-nonadien-3-ol, α-cyclohexylidene benzene acetonitrile, dec-9-en-1-ol, 2,6-dimethylheptan-1-ol, 3,7,7-trimethylbicyclo-[4.1.0].hept-3-ene, cis-4-(1-methylethyl)cyclohexanemethanol, 2-(1-methylpropyl)cyclohexanone, 4-(2,6,6-trimethylcyclohex-1-en-1-yl)-but-3-en-2-one, tetrahydro-4-methyl-2-(2-methyl-1-propenyl)-2H-pyran, 4-(2,6,6-trimethyl-1-cyclohexen-1-yl)butan-2-one, 2,6-dimethyl-2-octanol, tetrahydro-3-pentyl-2H-pyran-4-ylacetate, 2-methyldecanal, Seychelles, 2-trans-dodecanal, (4Z)-4-dodecenal, 5-methyl-3-heptanone oxime, tridec-2-enenitrile, 1-(2,6,6-trimethyl-1,3-cyclohexadien-1-yl)-2-buten-1-one, gyrane, hedione, (3Z)-3-hexen-1-ol-1-benzoate, oxydipropanol, 2,6-dimethyloct-7-en-2-yl formate, cyclohexyl ester-2-hydroxybenzoic acid, 4,4a,5,9b-tetrahydro-2,4-dimethylindeno-[1,2-d]-1,3-dioxin, dimethylcyclohex-3-en-1-carbaldehyde, 2-methyl-4-(2,2,3-trimethyl-3-cyclopenten-1-yl)-2-buten-1-ol, 2-ethyl-4-(2,2,3-trimethyl-3-cyclopenten-1-yl)-2-buten-1-ol, 7-methyl-2H-benzo-1,5-dioxepin-3(4H)-one, 4-(tricyclo-[5.2.1.02,6]-dec-8-ylidene)butyraldehyde, 4-tert-butylcyclohexyl acetate, 1,2,3,5,6,7-hexahydro-1,1,2,3,3-p-4H-inden-4-one, 5-cyclohexadecen-1-one, ethyl 2-methylvalerate, isononyl ester acetic acid, 1-(2,6,6-trimethyl-2-cyclohexen-1-yl)-2-buten-1-one, 2-methylbutyl salicylate, 3,7-dimethyloct-6-enenitrile, 1-methyl-3-(4-methyl-3-penten-1-yl)-3-cyclohexen-1-carboxaldehyde, 2-methyl-6-methylene-7-octen-2-ol (dihydroderivative), 1-(1,2,3,4,5,6,7,8-octahydro-2,3,8,8-tetramethyl-2-naphthyl)ethan-1-one, 3A,4,5,6,7,7A-hexahydro-4,7-methano-1H-inden-5(or 6)-ylacetate, γ-methylbenzenepentanol, 1-(5,5-dimethyl-1-cyclohexen-1-yl)pent-4-en-1-one, 1-(2,6,6-trimethyl-3-cyclohexen-1-yl)-2-buten-1-one, ethyl 2-ethyl-6,6-dimethylcyclohex-2-en-1-carboxylate, 8-undecenal, (ethoxymethoxy)cyclododecane, cis-2-methyl-4-propyl-1,3-oxathian, 3,7-dimethyl-2,6-nonadienenitrile, tetrahydro-4-methyl-2-(2-methylpropyl)-2H-pyran-4-ol, α,β-2,2,3-pentamethylcyclopent-3-en-1-butanol, (Z)-3-hexenyl salicylate, isopropyl-2-methylbutyrate, 3-(p-ethylphenyl)-2,2-dimethylpropionaldehyde, 6,6-dimethoxy-2,5,5-trimethylhex-2-ene, ethyl-3,5,5-trimethylhexanoate, 3-methyl-5-(2,2,3-trimethyl-3-cyclopenten-1-yl)-4-penten-2-ol, 2-tert-butylcyclohexylethyl carbonate, 3,6-dimethylcyclohex-3-en-1-carbaldehyde, 3,3,5-trimethylcyclohexanol-1-acetate, a,7,7-trimethylbenzenepropanol-1-acetate, vetiverol, 6(or 8)-(sec-butyl)quinoline, decene (hydroformylation products), sweet orange oil terpenes, bergamot oil (psoralen-free), allyl(cyclohexyloxy)acetate, 3a,4,5,6,7,7α-hexahydro-4,7-methano-1H-indenylpropionate, 1-(2,2,6-trimethylcyclohexyl)-3-hexanol, trimethyl-13-oxabicyclo-[10.1.0]-trideca-4,8-diene, diethyl ester-1,4-cyclohexanedicarboxylic acid, 2,4,4,7-tetramethyl-6-octen-3-one, 3-methyl-3-buten-1-yl 2,2-dimethylpropanoate, 4-methyl-3-decen-5-ol, 3-methylcyclopentadecenone, patchouli extract, eucalyptus oil, Boswellia carterii extract, 2-(2-(4-methyl-3-cyclohexen-1-yl)propyl)cyclopentanone, 3,3-dimethyl-5-(2,2,3-trimethyl-3-cyclopenten-1-yl)-4-penten-2-ol, (12E)-oxacyclohexadec-12-en-2-one, β-methyl-3-(1-methylethyl)-benzenepropanal, ethyloctahydro-4,7-methano-3aH-indene-3a-carboxylate, 2,4-dimethyl-2-(5,6,7,8-tetrahydro-5,5,8,8-tetramethyl-2-naphthalenyl)-1,3-dioxolane, 1-(2-tert-butylcyclohexyloxy)-2-butanol, 2-{1-(3,3-dimethylcyclohexyl)ethoxyl}-2-methyl-1-propanol, propalonone, cis-3-hexenylcyclopropionate, 1-methyl-2-[(1,2,2-trimethylbicyclo-[3.1.0]-hex-3-yl)methyl]cyclopropane methanol, (4aR,5R,7aS,9R)-octahydro-2,2,5,8,8,9a-hexamethyl-4H-4a,9-methanoazuleno-[5,6-d]-1,3-dioxole, 2,5-dimethyl-2,3-dihydro-1H-inden-2-yl)methanol, propyl-(2S)-2-(1,1-dimethylpropoxy)propionate, decahydro-2,2,6,6,7,8,8-heptamethyl-2H-indeno-[4,5-b]-furan, 3-(dodecylthio)-1-(2,6,6-trimethyl-3-cyclohexen-1-yl)-1-butanone, methyl-2,6,6-trimethylcyclohex-3-en-1-carboxylate, 3,7-dimethyl-6-octenylethyl ester silicic acid, (2E)-3,7-dimethyl-2,6-octadienylethyl ester silica, ethyl 2-phenylethyl ester silica and mixtures thereof.

In further preferred embodiments, the at least one fragrance is selected from the group consisting of 2-methyl-6-methylene-7-octen-2-ol (dihydro derivatives), 3A,4,5,6,7,7A-hexahydro-4,7-methano-1H-inden-5(or 6)-yl acetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, linalool, 4-(2,6,6-trimethylcyclohex-1-en-1-yl)-but-3-en-2-one, hedione, coumarin, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allylheptanoate, nerol, pin-2(3)-ene, diphenyl ether, citronellol, methyl 2-naphthyl ether, methyl benzoate, 5-heptyldihydro-2(3H)-furanone, isopropyl ester myristic acid, γ-decalactone, ethyl-2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, dodecanal, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, bornan-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-3-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehydeethylphenylethylacetal, β-4-dimethyl-3-cyclohexene-1-propanal, peppermint oil, d-p-mentha-1(6),8-dien-2-one, β-caryophyllene, octanal, lavender oil, (3z)-3-hexen-1-ol-1-benzoate, geraniol, decahydro-2,2,6,6,7,8,8-heptamethyl-2H-indeno-[4,5-b]-furan, 7-methyl-3-methyleneocta-1,6-diene, Seychelles, undec-10-enal, 3,7-dimethyl-1,6-octadien-3-ol acetate, cis-3-hexenol, dipentene (crude), 2-trans-dodecanal, longifolene, methyloct-2-ynoate, indole, camphene, p-menth-1-en-8-ol, p-mentha-1(7),2-diene, 8-undecenal, benzyl alcohol, pine oil, β-patchoulene, 3,7-dimethyloctan-1-ol, p-mentha-1,4(8)-diene, (4Z)-4-dodecenal, diethanolamine, butyl salicylate, farnesol, phosphoric acid, 4-allylanisole, benzyl salicylate and mixtures thereof.

In even more preferred embodiments, the at least one fragrance is selected from the group consisting of α-isomethyl ionone, benzyl salicylate, citronellol, coumarin, hexyl cinnamal, linalool, pentanoic acid, 2-methylethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-iron, β-phenylethylbenzoate, α-santalol, cedrol, cedryl acetate, cedryl formate, cyclohexyl salicyate, γ-dodecalactone, β-phenylethylphenylacetate, 2-methyl-6-methylene-7-octen-2-ol (dihydroderivatives), 3A,4,5,6,7,7A-hexahydro-4,7-methano-1H-inden-5(or 6)-ylacetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, 4-(2,6,6-trimethylcyclohex-1-en-1-yl)but-3-en-2-one, hedione, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allylheptanoate, nerol, pin-2(3)-ene, diphenyl ether, methyl 2-naphthyl ether, methyl benzoate, 5-heptyldihydro-2(3H)-furanone, isopropyl ester myristic acid, 7-decalactone, ethyl 2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethylphenyl ethyl acetal, b-4-dimethyl-3-cyclohexene-1-propanal, peppermint oil, d-p-mentha-1(6),8-dien-2-one and mixtures thereof.

In particularly preferred embodiments, the at least one fragrance is selected from the group consisting of 2-methyl-6-methylene-7-octen-2-ol (dihydro derivatives), 3A,4,5,6,7,7A-hexahydro-4,7-methano-1H-inden-5(or 6)-yl acetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, linalool, 4-(2,6,6-trimethylcyclohex-1-en-1-yl)but-3-en-2-one, hedione, coumarin, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allylheptanoate, nerol, pin-2(3)-ene, diphenyl ether, citronellol, methyl 2-naphthyl ether, methyl benzoate, 5-heptyldihydro-2(3H)-furanone, isopropyl ester myristic acid, γ-decalactone, ethyl-2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, dodecanal, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl 5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethyl phenyl ethyl acetal, b-4-dimethyl-3-cyclohexene-1-propanal, peppermint oil, d-p-mentha-1(6),8-dien-2-one and mixtures thereof.

In preferred embodiments, the at least one and increasingly preferably the at least two, three, four, five, six, seven or more fragrance(s) is/are selected from the group consisting of 2-phenylethanol, hexanal, bornan-2-one, 3,7-dimethyl-3-octanol, linalool, allyl ionone, 3-methyl-4-(2,6,6-trimethyl-1-cyclohexen-1-yl)-3-buten-2-one, camphene, p-menth-1-en-8-yl acetate, pin-2(3)-ene, methyl N-methylanthranilate, isobutyl salicylate, isopentyl salicylate, β-caryophyllene, 2-tert-butylcyclohexyl acetate, (1S-cis)-1,2,3,4,5,6,7,8-octahydro-7-isopropylidene-1,4-dimethylazulene, coumarin, methyl 2-naphthyl ether, 2′-acetonapthone, ethyl 2-naphthyl ether, methyl benzoate, 1-phenylethyl acetate, eugenol, 4-(1,1-dimethylethyl)cyclohexanol, p-menth-1-en-8-ol, acetophenone, d-p-mentha-1(6),8-dien-2-one, p-mentha-1,4-diene, benzyl alcohol, benzaldehyde, (2,2-dimethoxyethyl)benzene, diphenyl ether, α-hexylcinnamaldehyde, methyl 3-phenyl-2-propenoate, benzyl cinnamate, 2-phenylethyl acetate, phenoxyethyl isobutyrate, 3-p-cumenyl-2-methylpropionaldehyde, anethole, 5-heptyldihydro-2(3H)-furanone, 4-methylanisole, 4-methoxybenzyl alcohol, ethyl butyrate, 1,4-dioxacycloheptadecan-5,17-dione, oxacyclohexadecan-2-one, (E)-3,7-dimethyl-2,6-octadien-1-ol acetate, 3,7-dimethyloctan-1-ol, citronellol, geraniol, nerol, 2,6-dimethyl-5-heptenal, 7-hydroxycitronellal, oxacycloheptadecan-2-one, myristic acid isopropyl ester, 2-methylundecanal, methyloct-2-ynoate, hexan-1-ol, diethanolamine, methylnon-2-enoate, 2-undecanone, decan-1-ol, decanal, undecanal, undec-10-enal, dodecanal, 3,7-dimethyl-1,6-octadien-3-ol acetate, phenyl salicylate, benzyl salicylate, 3-hydroxy-2-methyl-4H-pyran-4-one, methyl salicylate, benzyl benzoate, 1,3-benzodioxole-5-carboxaldehyde, indole, 3-ethoxy-4-hydroxybenzaldehyde, 4-hydroxy-3-methoxy-benzaldehyde, 4′-methylacetophenone, 4-isopropylbenzaldehyde, 2-benzylidenheptanal, 2-phenoxyethanol, anisaldehyde, 7-methyl-3-methylene octa-1,6-diene, allyl hexanoate, isopentyl acetate, octanal, C9 aldehyde, exo-1,7,7-trimethylbicyclo-[2.2.1]-hept-2-yl acetate, 4-(2,6,6-trimethylcyclohex-2-en-1-yl)-but-3-en-2-one, 1-(2,6,6-trimethyl-1-cyclohexen-1-yl)pent-1-en-3-one, 3-methyl-4-(2,6,6-trimethyl-2-cyclohexen-1-yl)-3-buten-2-one, pin-2(10)-ene, 2,6-di-tert-butyl-p-cresol, methyl anthranilate, 2-heptylcyclopentanone, dipentene (crude), phenylmethylesteracetic acid, 4-allylanisole, (2Z)-3,7-dimethyl-2,6-octadien-1-ol-1-acetate, 2,6,10-trimethyl-9-undecenal, 2-aminoethanol, allyl heptanoate, hexyl acetate, 9-undecenal, citronellyl acetate, α-, α-dimethylphenethyl acetate, cineole, 2,6,6-trimethylbicyclo-[3.1.1]-heptane, longifolene, cyclopentadecanone, DL-borneol, β-patchoulene, p-mentha-1(7),2-diene, (2E,6Z)-nona-2,6-dien-1-al, p-mentha-1,4(8)-diene, γ-decalactone, cis-3-hexenol, (1R,4R,6R,10S)-4,12,12-trimethyl-9-methylene-5-oxatricyclo-[8.2.0.04,6]-dodecane, 3-methyl-2-buten-1-ol acetate, methyl ionone, pentyl salicylate, butyl salicylate, dodecanonitrile, 4,7-dimethyl-6-octen-3-one, 4-phenylbutan-2-one, acetaldehydeethylphenylethyl acetal, 2-methoxy-4-propylphenol, ethyl 2-ethylhexanoate, (Z)-3,7-dimethylocta-1,3,6-triene, α-butylbenzeneacetonitrile, phenylethyl methyl ether, (Z)-3-hexen-1-ol acetate, octahydro-2H-1-benzopyran-2-one, farnesol, 5-methyl-5-phenylhexan-3-one, 2-ethyl-3-hydroxy-4-pyrone, 2,4,6-trimethyl-4-phenyl-1,3-dioxane, citral, 3-(p-methoxyphenyl)-2-methylpropionaldehyde, 4-(4-hydroxyphenyl)butan-2-one, patchouli alcohol, tetrahydrocitral, (R)-p-mentha-1,8-diene, hexyl salicylate, 1-p-mentha-1(6),8-dien-2-one, trans-hex-2-enal, β-4-dimethyl-3-cyclohexene-1-propanal, [3aR-(3aa,5ab,9aa,9bb)]-dodecahydro-3a,6,6,9α-tetramethylnaphtho-[2,1-b]-furan, ethyl 2-methylbutyrate, [2-(1-propoxyethoxy)ethyl]benzene, phosphoric acid, 1-(2,6,6-trimethyl-2-cyclohexen-1-yl)pent-1-en-3-one, oxacycloheptadec-7-en-2-one, terpineol, pine oil, mandarin oil, orange oil (sweet), peppermint oil, ionone, patchouli oil, parsley oil, vetiver oil, lavender oil, resin acids & rosin acids (hydrogenated, Me-esters), benzoin resin, α,α-dimethylphenethylbutyrate, 3,7-dimethyl-1,6-nonadien-3-ol, α-cyclohexylidene benzene acetonitrile, dec-9-en-1-ol, 2,6-dimethylheptan-1-ol, 3,7,7-trimethylbicyclo-[4.1.0].hept-3-ene, cis-4-(1-methylethyl)cyclohexanemethanol, 2-(1-methylpropyl)cyclohexanone, 4-(2,6,6-trimethylcyclohex-1-en-1-yl)-but-3-en-2-one, tetrahydro-4-methyl-2-(2-methyl-1-propenyl)-2H-pyran, 4-(2,6,6-trimethyl-1-cyclohexen-1-yl)butan-2-one, 2,6-dimethyl-2-octanol, tetrahydro-3-pentyl-2H-pyran-4-ylacetate, 2-methyldecanal, Seychelles, 2-trans-dodecanal, (4Z)-4-dodecenal, 5-methyl-3-heptanone oxime, tridec-2-enenitrile, 1-(2,6,6-trimethyl-1,3-cyclohexadien-1-yl)-2-buten-1-one, gyrane, hedione, (3Z)-3-hexen-1-ol-1-benzoate, oxydipropanol, 2,6-dimethyloct-7-en-2-yl formate, cyclohexyl ester-2-hydroxybenzoic acid, 4,4a,5,9b-tetrahydro-2,4-dimethylindeno-[1,2-d]-1,3-dioxin, dimethylcyclohex-3-en-1-carbaldehyde, 2-methyl-4-(2,2,3-trimethyl-3-cyclopenten-1-yl)-2-buten-1-ol, 2-ethyl-4-(2,2,3-trimethyl-3-cyclopenten-1-yl)-2-buten-1-ol, 7-methyl-2H-benzo-1,5-dioxepin-3(4H)-one, 4-(tricyclo-[5.2.1.02,6]-dec-8-ylidene)butyraldehyde, 4-tert-butylcyclohexyl acetate, 1,2,3,5,6,7-hexahydro-1,1,2,3,3-p-4H-inden-4-one, 5-cyclohexadecen-1-one, ethyl 2-methylvalerate, isononyl ester acetic acid, 1-(2,6,6-trimethyl-2-cyclohexen-1-yl)-2-buten-1-one, 2-methylbutyl salicylate, 3,7-dimethyloct-6-enenitrile, 1-methyl-3-(4-methyl-3-penten-1-yl)-3-cyclohexen-1-carboxaldehyde, 2-methyl-6-methylene-7-octen-2-ol (dihydroderivative), 1-(1,2,3,4,5,6,7,8-octahydro-2,3,8,8-tetramethyl-2-naphthyl)ethan-1-one, 3A,4,5,6,7,7A-hexahydro-4,7-methano-1H-inden-5(or 6)-ylacetate, 7-methylbenzenepentanol, 1-(5,5-dimethyl-1-cyclohexen-1-yl)pent-4-en-1-one, 1-(2,6,6-trimethyl-3-cyclohexen-1-yl)-2-buten-1-one, ethyl 2-ethyl-6,6-dimethylcyclohex-2-en-1-carboxylate, 8-undecenal, (ethoxymethoxy)cyclododecane, cis-2-methyl-4-propyl-1,3-oxathian, 3,7-dimethyl-2,6-nonadienenitrile, tetrahydro-4-methyl-2-(2-methylpropyl)-2H-pyran-4-ol, α,β-2,2,3-pentamethylcyclopent-3-en-1-butanol, (Z)-3-hexenyl salicylate, isopropyl 2-methylbutyrate, 3-(p-ethylphenyl)-2,2-dimethylpropionaldehyde, 6,6-dimethoxy-2,5,5-trimethylhex-2-ene, ethyl-3,5,5-trimethylhexanoate, 3-methyl-5-(2,2,3-trimethyl-3-cyclopenten-1-yl)-4-penten-2-ol, 2-tert-butylcyclohexylethyl carbonate, 3,6-dimethylcyclohex-3-en-1-carbaldehyde, 3,3,5-trimethylcyclohexanol-1-acetate, a,7,7-trimethylbenzenepropanol-1-acetate, vetiverol, 6(or 8)-(sec-butyl)quinoline, decene (hydroformylation products), sweet orange oil terpenes, bergamot oil (psoralen-free), allyl(cyclohexyloxy)acetate, 3a,4,5,6,7,7α-hexahydro-4,7-methano-1H-indenylpropionate, 1-(2,2,6-trimethylcyclohexyl)-3-hexanol, trimethyl-13-oxabicyclo-[10.1.0]-trideca-4,8-diene, diethyl ester-1,4-cyclohexanedicarboxylic acid, 2,4,4,7-tetramethyl-6-octen-3-one, 3-methyl-3-buten-1-yl 2,2-dimethylpropanoate, 4-methyl-3-decen-5-ol, 3-methylcyclopentadecenone, patchouli extract, eucalyptus oil, Boswellia carterii extract, 2-(2-(4-methyl-3-cyclohexen-1-yl)propyl)cyclopentanone, 3,3-dimethyl-5-(2,2,3-trimethyl-3-cyclopenten-1-yl)-4-penten-2-ol, (12E)-oxacyclohexadec-12-en-2-one, β-methyl-3-(1-methylethyl)-benzenepropanal, ethyloctahydro-4,7-methano-3aH-indene-3a-carboxylate, 2,4-dimethyl-2-(5,6,7,8-tetrahydro-5,5,8,8-tetramethyl-2-naphthalenyl)-1,3-dioxolane, 1-(2-tert-butylcyclohexyloxy)-2-butanol, 2-{1-(3,3-dimethylcyclohexyl)ethoxyl}-2-methyl-1-propanol, propalonone, cis-3-hexenylcyclopropionate, 1-methyl-2-[(1,2,2-trimethylbicyclo-[3.1.0]-hex-3-yl)methyl]cyclopropane methanol, (4aR,5R,7aS,9R)-octahydro-2,2,5,8,8,9α-hexamethyl-4H-4a,9-methanoazuleno-[5,6-d]-1,3-dioxole, 2,5-dimethyl-2,3-dihydro-1H-inden-2-yl)methanol, propyl-(2S)-2-(1,1-dimethylpropoxy)propionate, decahydro-2,2,6,6,7,8,8-heptamethyl-2H-indeno-[4,5-b]-furan, 3-(dodecylthio)-1-(2,6,6-trimethyl-3-cyclohexen-1-yl)-1-butanone, methyl-2,6,6-trimethylcyclohex-3-en-1-carboxylate, 3,7-dimethyl-6-octenylethyl ester silicic acid, (2E)-3,7-dimethyl-2,6-octadienylethyl ester silica, ethyl 2-phenylethyl ester silica and mixtures thereof.

In further preferred embodiments, the at least one and increasingly preferably the at least two, three, four, five, six, seven or more fragrance(s) is/are selected from the group consisting of 2-methyl-6-methylene-7-octen-2-ol (dihydro derivatives), 3A,4,5,6,7,7A-hexahydro-4,7-methano-1H-inden-5(or 6)-yl acetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, linalool, 4-(2,6,6-trimethylcyclohex-1-en-1-yl)-but-3-en-2-one, hedione, coumarin, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allyl heptanoate, nerol, pin-2(3)-ene, diphenyl ether, citronellol, methyl 2-naphthyl ether, methyl benzoate, 5-heptyldihydro-2(3H)-furanone, isopropyl ester myristic acid, γ-decalactone, ethyl-2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, dodecanal, DL-borneol, eugenol, ethyl 2-methylbutyrate, 1,3-dimethyl-3-butenyl-2-methylpropionate, 4-methylanisole, 3-phenyl-2-propenoic acid methyl ester, patchouli alcohol, 9-undecenal, bornan-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-3-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehydeethylphenylethyl acetal, β-4-dimethyl-3-cyclohexene-1-propanal, peppermint oil, d-p-mentha-1(6),8-dien-2-one, β-caryophyllene, octanal, lavender oil, (3z)-3-hexen-1-ol-1-benzoate, geraniol, decahydro-2,2,6,6,7,8,8-heptamethyl-2H-indeno-[4,5-b]-furan, 7-methyl-3-methyleneocta-1,6-diene, Seychelles, undec-10-enal, 3,7-dimethyl-1,6-octadien-3-ol acetate, cis-3-hexenol, dipentene (crude), 2-trans-dodecanal, longifolene, methyl oct-2-ynoate, indole, camphene, p-menth-1-en-8-ol, p-mentha-1(7),2-diene, 8-undecenal, benzyl alcohol, pine oil, β-patchoulene, 3,7-dimethyloctan-1-ol, p-mentha-1,4(8)-diene, (4Z)-4-dodecenal, diethanolamine, butyl salicylate, farnesol, phosphoric acid, 4-allylanisole, benzyl salicylate and mixtures thereof.

In particularly preferred embodiments, the at least one and increasingly preferably the at least two, three, four, five, six, seven or more fragrance(s) is/are selected from the group consisting of 2-methyl-6-methylene-7-octen-2-ol (dihydro derivatives), 3A,4,5,6,7,7A-hexahydro-4,7-methano-1H-inden-5(or 6)-yl acetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, linalool, 4-(2,6,6-trimethylcyclohex-1-en-1-yl)but-3-en-2-one, hedione, coumarin, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7α-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allyl heptanoate, nerol, pin-2(3)-ene, diphenyl ether, citronellol, methyl 2-naphthyl ether, methyl benzoate, 5-heptyldihydro-2(3H)-furanone, isopropyl ester myristic acid, γ-decalactone, ethyl-2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, dodecanal, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethylphenyl ethyl acetal, b-4-dimethyl-3-cyclohexen-1-propanal, peppermint oil, d-p-mentha-1(6),8-dien-2-one and mixtures thereof.

Within the context of the invention, cleaning performance is understood to mean the ability of an agent to partially or completely remove an existing stain, in particular the lightening performance on one or more stains on textiles. Within the context of the invention, both the agent which comprises enzymes or the washing liquor formed by this agent and the corresponding enzyme itself have a corresponding cleaning performance. The cleaning performance of the enzyme thus contributes to the cleaning performance of the agent or the washing liquor formed by the agent. The cleaning performance is preferably ascertained as described hereafter.

Washing liquor is understood to mean the use solution containing the washing agent which acts on the textiles or fabrics and thus comes into contact with the stains present on the textiles or fabrics. The washing liquor is usually formed when the washing process begins and the washing agent is diluted with water, for example in a washing machine or in another suitable container.

The cleaning performance can be determined in a washing system containing a washing agent in a dosage between 2.0 and 8.0 grams per liter of washing liquor and the enzyme to be examined. Enzymes to be compared are used in the same concentration (based on active protein). The activity-equivalent use of the enzymes ensures that the respective enzymatic properties, for example the cleaning performance on certain stains, are compared even if the ratio of active substance to total protein (the values of the specific activity) diverges. In general, a low specific activity can be compensated by adding a larger amount of protein. Furthermore, the enzymes to be examined can also be used in the same amount of substance or amount by weight if the enzymes to be examined have a different affinity for the test substrate in an activity test. The expression “same amount of substance” in this context relates to a molar use of the enzymes to be examined. The expression “equal weight” relates to the use of the same weight of the enzymes to be examined.

The concentration of the enzyme in the washing agent intended for this washing system is 0.0001 to 0.1 wt. %, in particular 0.001 to 0.1 wt. %, more preferably 0.01 to 0.06 wt. % and particularly preferably 0.001 to 0.02 wt. %, based on active protein.

The protein concentration can be determined using known methods, for example the BCA method (bicinchoninic acid; 2,2′-bichinolyl-4,4′-dicarboxylic acid) or the Biuret method (Gornall at al., J. Biol. Chem. 1948, 177: 751-766). In this regard, the active protein concentration can be determined via titration of the active centers using a suitable irreversible inhibitor and determination of the residual activity (cf. Bender et al., J. Am. Chem. Soc. 1966, 88, 24: 5890-5913).

A liquid reference washing agent for such a washing system may be composed, for example, as follows (all figures in percent by weight (wt. %)): 4.4% alkyl benzene sulfonic acid, 5.6% other anionic surfactants, 2.4% C₁₂₋₁₈ Na salts of fatty acids (soaps), 4.4% non-ionic surfactants, 0.2% phosphonates, 1.4% citric acid, 0.95% NaOH, 0.01% defoamer, 2% glycerol, 0.08% preservatives, 1% ethanol, and the remainder being demineralized water. The dosage of the liquid washing agent is preferably between 3.0 and 6.0 grams per liter of washing liquor, for example 3.0, 3.2, 3.5, 3.7, 4.0, 4.5, 4.7, 4.9 or 5.9 grams per liter of washing liquor. The washing process preferably takes place in a pH range between pH 7 and pH 10.5, preferably between pH 8 and pH 9.

The cleaning performance against stains on textiles is determined by measuring the degree of cleaning of the washed textiles. For example, the washing process can take place for 60 minutes at a temperature of 40° C. and the water can have a water hardness between 15.5°dH and 16.5°dH (German hardness).

The degree of whiteness, i.e., the lightening of the stains, as a measure of the cleaning performance is determined using optical measuring methods, preferably photometrically. A suitable device for this is, for example, the Minolta CM508d spectrometer. Usually, the devices used for measurement are calibrated beforehand using a white standard, preferably a supplied white standard.

Preferred embodiments of agents according to the invention achieve such advantageous cleaning performance even at low temperatures, in particular in the temperature ranges between 10° C. and 60° C., preferably between 15° C. and 50° C., more preferably between 20° C. and 40° C., and particularly preferably at 30° C.

This washing and cleaning agents according to the invention cover all conceivable types of washing or cleaning agents, both concentrates and undiluted agents, for use on a commercial scale, in washing machines or for hand washing or cleaning. These include, for example, washing agents for textiles, carpets or natural fibers for which the term washing agent is used. These include, for example, dishwashing detergents for dishwashers (automatic dishwasher detergent) or manual dishwashing detergents or cleaners for hard surfaces such as metal, glass, porcelain, ceramics, tiles, stone, painted surfaces, plastics, wood or leather, for which the term cleaning agent is used, i.e., in addition to manual and automatic dishwashing detergents, also, for example, scouring agents, glass cleaners, toilet scenters, etc. The washing and cleaning agents according to the invention also include auxiliary washing agents which are added to the actual washing agent during manual or automatic textile washing in order to achieve a further effect. Furthermore, in the context of the invention, washing and cleaning agents also include textile pre-treatment agents and post-treatment agents, i.e., those agents with which the item of laundry is brought into contact before the actual washing, for example for dissolving stubborn stains, and also those agents which give the laundry further desirable properties, such as a pleasant feel, crease resistance or a low static charge in a step downstream of the actual textile washing. Inter alia, softeners are included in the latter agents. This also comprises agents for use in (semi-)automated washing or cleaning systems such as floor-mopping robots or wet vacuum cleaners.

The washing and/or cleaning agents according to the invention, which may be in the form of powdered or granular solids, in compacted or further-compacted particulate form, homogeneous solutions or suspensions, may contain, in addition to the enzymes according to the invention, all known ingredients conventional in such agents, with preferably at least one other ingredient being present in the agent. In particular, the agents according to the invention can be surfactants, builders, complexing agents, polymers, glass corrosion inhibitors, corrosion inhibitors, bleaching agents such as peroxygen compounds, bleach activators or bleach catalysts, water-miscible organic solvents, enzyme stabilizers, sequestering agents, electrolytes, pH regulators and/or other additives such as optical brighteners, graying inhibitors, dye transfer inhibitors, foam regulators as well as dyes and fragrances. Advantageous ingredients of agents according to the invention are disclosed in international patent application WO 2009/121725, starting at the penultimate paragraph of page 5 and ending after the second paragraph on page 13. Reference is expressly made to this disclosure and the disclosure therein is incorporated into the present patent application.

An agent according to the invention advantageously contains the enzymes according to the invention in an amount of 2 μg to 20 mg, preferably of 5 μg to 17.5 mg, particularly preferably of 20 μg to 15 mg and very particularly preferably of 50 μg to 10 mg per g of the agent.

An agent according to the invention contains the enzymes according to the invention in an amount, increasingly preferably, of in each case 1×10⁻⁸ to 5 wt. %, of 0.0001 to 1 wt. %, of 0.0005 to 0.5 wt. %, of 0.001 to 0.1 wt. %, in each case based on active protein and based on the total weight of the agent.

In preferred embodiments, a washing and/or cleaning agent according to the invention contains the dispersin in an amount of 0.01 to 5 wt. %, preferably 0.1 to 4 wt. %, particularly preferably 0.15 to 2.5 wt. %, based on the total weight of the agent.

In preferred embodiments, a washing and/or cleaning agent according to the invention contains the at least three further enzymes, which are selected from among amylase, protease, lipase, cellulase, mannanase, pectate lyase and mixtures thereof, in a total amount of from 0.01 to 10 wt. %, preferably from 0.1 to 8 wt. %, particularly preferably from 0.2 to 6 wt. %, based on the total weight of the agent.

Furthermore, the enzymes contained in the agent, and/or other ingredients of the agent, may be coated with a substance which is impermeable to the enzyme at room temperature or in the absence of water, and which becomes permeable to the enzyme under conditions of use of the agent. Such an embodiment of the invention is thus characterized in that the enzyme is coated with a substance which is impermeable to the enzyme at room temperature or in the absence of water. Furthermore, the agent itself can be packed in a container, preferably an air-permeable container, from which it is released shortly before use or during the washing process.

Further embodiments that comprise all solid, powdery, liquid, gel-like or pasty dosage forms of agents according to the invention, which may optionally also consist of a plurality of phases and may be present in compressed or non-compressed form. The agent can be present as a free-flowing powder, in particular having a bulk density of 300 g/l to 1200 g/1, in particular 500 g/l to 900 g/l or 600 g/l to 850 g/1. The solid administration forms of the agent further include extrudates, granules, tablets or pouches. Alternatively, the agent can also be in a liquid, gel or paste form, for example in the form of a non-aqueous liquid washing agent or a non-aqueous paste or in the form of an aqueous liquid washing agent or a water-containing paste. Liquid agents are generally preferred. Furthermore, the agent can be present as a single-component system. Such agents consist of one phase. Alternatively, an agent can also consist of a plurality of phases. Such an agent is accordingly divided into a plurality of components.

In a preferred embodiment, the agent according to the invention is a textile washing agent.

In a more preferred embodiment, the agent according to the invention is a liquid textile washing agent.

In a particularly preferred embodiment, the agent according to the invention is a pre-portioned washing agent, in particular a washing agent portion unit comprising a washing agent preparation according to the invention and a water-soluble film which completely encloses the washing agent preparation.

The water-soluble film in which the washing agent preparation is packaged can comprise one or more structurally different water-soluble polymer(s). Suitable water-soluble polymer(s) are in particular polymers from the group of (optionally acetalized) polyvinyl alcohols (PVAL) and copolymers thereof. Water-soluble films are preferably based on a polyvinyl alcohol or a polyvinyl alcohol copolymer whose molecular weight is in the range of from 10000 to 1,000,000 g/mol, preferably from 20000 to 500000 g/mol, particularly preferably from 30000 to 100000 g/mol, and in particular from 40000 to 80000 g/mol. Suitable water-soluble films for use are marketed, inter alia, by the company monosol LLC, for example under the name M8630, M8720, M8310, C8400 or M8900. For example, films with the name Solublon® PT, Solublon® GA, Solublon® KC or Solublon® KL by Aicello Chemical Europe GmbH or the VF-HP films by Kuraray are also suitable.

If the washing agents according to the invention are in liquid form, they preferably contain more than 40 wt. %, preferably 50 to 90 wt. %, and particularly preferably 60 to 80 wt. %, water, based on their total weight.

The agents according to the invention can contain one or more surfactants, with anionic surfactants, non-ionic surfactants, and mixtures thereof being particularly suitable, although cationic, zwitterionic, and/or amphoteric surfactants can also be contained. The agents preferably contain 5 to 70 wt. % surfactant, preferably 35 to 60 wt. %, and more preferably 40 to 55 wt. % surfactant.

Suitable anionic surfactants are, in particular, soaps and those which contain sulfate or sulfonate groups, preferably having alkali ions as cations. Usable soaps are preferably the alkali salts of saturated or unsaturated C₁₂₋₁₈ fatty acids. Fatty acids of this kind can also be used in a not completely neutralized form. Suitable sulfate-type surfactants include the salts of sulfuric acid semiesters of C₁₂₋₁₈ fatty alcohols and the sulfation products of the mentioned non-ionic surfactants having a low degree of ethoxylation. Surfactants of the sulfonate type that can be used include, for example, C₉₋₁₄ alkylbenzene sulfonates, alkane sulfonates obtained from C₁₂₋₁₈ alkanes, for example by sulfochlorination or sulfoxidation with subsequent hydrolysis or neutralization, C₁₂₋₁₈ olefin sulfonates resulting from the reaction of corresponding monoolefins with sulfur trioxide, mixtures of alkene and hydroxyalkane sulfonates, disulfonates, such as those obtained from C₁₂₋₁₈ monoolefins with terminal or internal double bonds by sulfonation with gaseous sulfur trioxide and subsequent alkaline or acidic hydrolysis of the sulfonation products, and α-sulfo fatty acid esters (ester sulfonates) resulting from the sulfonation of fatty acid methyl or ethyl esters, e.g., α-sulfonated methyl esters of hydrogenated coconut, palm kernel or tallow fatty acids.

The agent preferably comprises 2 to 55 wt. %, and more preferably 3 to 35 wt. %, anionic surfactant. Very particularly preferably, the agent comprises 3 to 25 wt. % alkylbenzene sulfonate. In addition, the agent can preferably also contain other anionic surfactants, in particular alkyl ether sulfates, and non-ionic surfactants, in particular fatty alcohol alkoxylates. These can then make up the remainder of the surfactants.

Suitable alkylbenzene sulfonates are preferably selected from linear or branched alkylbenzene sulfonates of the formula

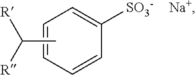

in which R′ and R″ are, independently of one another, hydrogen or alkyl, and together contain 6 to 19, preferably 7 to 15, and in particular 9 to 13, C atoms. A very particularly preferred representative is sodium dodecylbenzene sulfonate.

The alkali salts and in particular the sodium salts of the sulfuric acid semiesters of C₁₂₋₁₈ fatty alcohols, e.g., from coconut fatty alcohol, tallow fatty alcohol, lauryl alcohol, myristyl alcohol, cetyl alcohol or stearyl alcohol, or of C₁₀₋₂₀ oxo alcohols and the semiesters of secondary alcohols having these chain lengths are preferred as alk(en)yl sulfates. Alk(en)yl sulfates of the mentioned chain length that contain a synthetic straight-chain alkyl functional group prepared on a petrochemical basis and have a degradation behavior similar to that of the adequate compounds based on fat chemical raw materials are also preferred. From a washing perspective, the C₁₂₋₁₆ alkyl sulfates and C₁₂₋₁₅ alkyl sulfates and also C₁₄₋₁₅ alkyl sulfates are preferred.

The sulfuric acid monoesters of straight-chain or branched C₇₋₂₁ alcohols ethoxylated with 1 to 6 mol ethylene oxide, such as 2-methyl-branched C₉₋₁₁ alcohols with, on average, 3.5 mol ethylene oxide (EO) or C₁₂₋₁₈ fatty alcohols with 1 to 4 EO, are also suitable.

Suitable alkyl ether sulfates are, for example, compounds of the formula

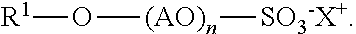

In this formula, R¹ represents a linear or branched, substituted or unsubstituted alkyl functional group, preferably a linear, unsubstituted alkyl functional group, particularly preferably a fatty alcohol functional group. Preferred functional groups R¹ are selected from decyl, undecyl, dodecyl, tridecyl, tetradecyl, pentadecyl, hexadecyl, heptadecyl, octadecyl, nonadecyl, eicosyl functional groups and mixtures thereof, the representatives having an even number of C atoms being preferred. Particularly preferred functional groups R¹ are derived from C₁₂₋₁₈ fatty alcohols, for example from coconut fatty alcohol, tallow fatty alcohol, lauryl, myristyl, cetyl or stearyl alcohol or from C10-20 oxo alcohols. AO represents an ethylene oxide (EO) group or propylene oxide (PO) group, preferably an ethylene oxide group. The index n represents an integer from 1 to 50, preferably from 1 to 20, and in particular from 2 to 10. Very particularly preferably, n represents the numbers 2, 3, 4, 5, 6, 7 or 8. X⁺ represents a monovalent cation or the n-th part of an n-valent cation, the alkali metal ions, including Na⁺ or K⁺, being preferred in this case, with Na⁺ being most preferred. Further cations X⁺ may be selected from NH₄⁺, ½ Zn²⁺, ½ Mg²⁺, ½ Ca²⁺, ½ Mn²⁺, and the mixtures thereof.

In various embodiments, the alkyl ether sulfate can be selected from fatty alcohol ether sulfates of the formula

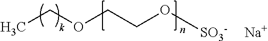

where k=11 to 19, and n=2, 3, 4, 5, 6, 7 or 8. Very particularly preferred representatives are Na—C₁₂₋₁₄ fatty alcohol ether sulfates having 2 EO (k=11-13, n=2). The degree of ethoxylation indicated represents a statistical average value which can be an integer or a fractional number for a specific product. The degrees of alkoxylation indicated represent statistical averages which can be an integer or a fractional number for a specific product. Preferred alkoxylates/ethoxylates have a narrowed homolog distribution (narrow range ethoxylates, NRE).

It has proven advantageous for cold wash performance if the agents additionally contain soap(s). Preferred agents are therefore characterized in that they contain soap(s). Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

Suitable non-ionic surfactants are in particular alkyl glycosides and ethoxylation and/or propoxylation products of alkyl glycosides or linear or branched alcohols each having 8 to approximately 18 C atoms in the alkyl portion and 3 to 20, preferably 4 to 10, alkyl ether groups.

Furthermore, corresponding ethoxylation and/or propoxylation products of N-alkylamines, vicinal diols, fatty acid esters, and fatty acid amides, which correspond to the above-mentioned long-chain alcohol derivatives with respect to the alkyl moiety, and of alkylphenols having 5 to 12 C atoms in the alkyl radical, may be used.

Non-ionic surfactants that are preferably used are alkoxylated, advantageously ethoxylated, in particular primary alcohols having preferably 8 to 18 C atoms and, on average, 1 to 12 mols of ethylene oxide (EO) per mol of alcohol, in which the alcohol functional group can be linear or preferably methyl-branched in position 2, or can contain linear and methyl-branched functional groups in admixture, as are usually present in oxo alcohol functional groups. However, alcohol ethoxylates having linear functional groups of alcohols of native origin having 12 to 18 C atoms, for example of coconut, palm, tallow fatty or oleyl alcohol, and an average of 2 to 8 EO per mol of alcohol, are particularly preferred. Preferred ethoxylated alcohols include, for example, C₁₂₋₁₄ alcohols having 3 EO or 4 EO, C₉₋₁₁ alcohols having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation specified represent statistical averages that can correspond to an integer or a fractional number for a specific product. Preferred alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of non-ionic surfactants that are preferably used, which are used either as the sole non-ionic surfactant or in combination with other non-ionic surfactants, is alkoxylated, preferably ethoxylated or ethoxylated and propoxylated fatty acid alkyl esters, preferably having 1 to 4 carbon atoms in the alkyl chain, in particular fatty acid methyl esters.

Another class of non-ionic surfactants that can advantageously be used is the alkyl polyglycosides (APG). Usable alkyl polyglycosides satisfy the general formula

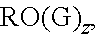

in which R is a linear or branched, in particular methyl-branched in the 2-position, saturated or unsaturated, aliphatic functional group having 8 to 22, preferably 12 to 18 C atoms and G is the symbol which represents a glycose unit having 5 or 6 C atoms, preferably glucose. The degree of glycosidation z is between 1 and 4, preferably between 1 and 2, and in particular between 1.1 and 1.4. Linear alkyl polyglycosides, i.e., alkyl polyglycosides in which the polyglycol functional group is a glucose functional group and the alkyl functional group is an n-alkyl functional group, are preferably used.

Non-ionic surfactants of the aminoxide type, for example N-cocoalkyl-N,N-dimethylamine oxide and N-tallow-alkyl-N,N-dihydroxyethylamine oxide, and of the fatty acid alkanolamides may also be suitable. The quantity of these non-ionic surfactants is preferably no more than that of the ethoxylated fatty alcohols, in particular no more than half thereof.

Suitable amphoteric surfactants are, for example, betaines of the formula

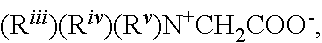

in which Rⁱⁱⁱ denotes an alkyl group, which is optionally interrupted by heteroatoms or heteroatom groups, having 8 to 25, preferably 10 to 21, carbon atoms, and R^{iv} and R^{v} denote identical or different alkyl functional groups having 1 to 3 carbon atoms, in particular C₁₀₋₁₈ alkyl dimethyl carboxymethyl betaine and C₁₁₋₁₇ alkyl amidopropyl dimethyl carboxymethyl betaine.

Suitable cationic surfactants include the quaternary ammonium compounds of the formula

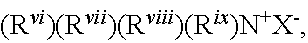

in which R^{vi} to R^{ix} represent four identical or different, in particular two long-chain and two short-chain, alkyl functional groups, and X⁻ represents an anion, in particular a halide ion, e.g., didecyldimethylammonium chloride, alkylbenzyldidecylammonium chloride, and mixtures thereof. Further suitable cationic surfactants are the quaternary surface-active compounds, in particular having a sulfonium, phosphonium, iodonium or arsonium group, which are also known as antimicrobial active ingredients. By using quaternary surface-active compounds having an antimicrobial effect, the agent can be provided with an antimicrobial effect or the antimicrobial effect that may already be present due to other ingredients can be improved.

Complexing agents are another preferred component of agents according to the invention. Particularly preferred complexing agents are the phosphonates, provided that their use is permitted by regulations. In addition to 1-hydroxyethane-1,1-diphosphonic acid, the complexing phosphonates include a number of different compounds such as diethylenetriamine penta(methylene phosphonic acid) (DTPMP). Hydroxyalkane or aminoalkane phosphonates are particularly preferred in this application. Among the hydroxyalkane phosphonates, 1-hydroxyethane-1,1-diphosphonate (HEDP) is of particular importance as a cobuilder. It is preferably used as a sodium salt, the disodium salt reacting neutrally and the tetrasodium salt reacting alkaline (pH 9). Possible amino alkane phosphonates preferably include ethylenediamine tetramethylene phosphonate (EDTMP), diethylenetriamine pentamethylene phosphonate (DTPMP) and the higher homologs thereof. They are preferably used in the form of the neutrally reacting sodium salts, e.g., as hexasodium salt of EDTMP or as the heptasodium and octasodium salt of DTPMP. Of the class of phosphonates, HEDP is preferably used as a builder. The aminoalkane phosphonates also have a pronounced heavy metal binding capacity.

Accordingly, it may be preferred, in particular if the agents also contain bleach, to use aminoalkane phosphonates, in particular DTPMP, or to use mixtures of the aforementioned phosphonates. A preferred agent in the context of this application contains one or more phosphonate(s) from the group aminotrimethylene phosphonic acid (ATMP) and/or the salts thereof; ethylenediamine tetra(methylene phosphonic acid) (EDTMP) and/or the salts thereof; diethylenetriamine penta(methylene phosphonic acid) (DTPMP) and/or the salts thereof; 1-hydroxyethane-1,1-diphosphonic acid (HEDP) and/or the salts thereof; 2-phosphonobutane-1,2,4-tricarboxylic acid (PBTC) and/or the salts thereof; hexamethylenediamine tetra(methylene phosphonic acid) (HDTMP) and/or the salts thereof; nitrilotri(methylenephosphonic acid) (NTMP) and/or the salts thereof. Agents containing 1-hydroxyethane-1,1-diphosphonic acid (HEDP) or diethylenetriaminepenta(methylenephosphonic acid) (DTPMP) as phosphonates are particularly preferred. The agent according to the invention may, of course, contain two or more different phosphonates.

Agents that are preferred according to the invention are characterized in that the agent contains at least one complexing agent from the group of phosphonates, preferably 1-hydroxyethane-1,1-diphosphonate, the proportion by weight of the phosphonate with respect to the total weight of the agent preferably being between 0.1 and 8.0 wt. %, more preferably 0.2 and 5.0 wt. %, even more preferably 0.3 and 3.0 wt. %, and particularly preferably 0.5-2.0 wt. %.

In further preferred embodiments, the washing and/or cleaning agents according to the invention are substantially free of phosphonate-containing compounds. “Substantially free of phosphonate-containing compounds” in this context means that the corresponding agents contain less than 2 wt. %, preferably less than 1 wt. %, more preferably less than 0.5 wt. %, and particularly preferably less than 0.1 wt. %, of phosphonate-containing compounds, based on the total weight of the agent. In particularly preferred embodiments, these agents are free of phosphonate-containing compounds.

The agents according to the invention also preferably contain builders, preferably at least one water-soluble and/or water-insoluble, organic, and/or inorganic builder. The builders include in particular the silicates, carbonates and/organic cobuilders.

Polycarboxylates/polycarboxylic acids, polymeric polycarboxylates, aspartic acid, polyacetals, dextrins, other organic cobuilders and phosphonates are particularly noteworthy as organic cobuilders. These substance classes are described below. Organic cobuilder substances of this kind can, if desired, be contained in amounts of up to 40 wt. %, in particular up to 25 wt. %, and preferably of 1 to 8 wt. %. Suitable organic builder substances are, for example, the polycarboxylic acids that can be used in the form of the free acids and/or the sodium salts thereof, where polycarboxylic acids are understood to mean the carboxylic acids which carry more than one acid function. For example, these are citric acid, adipic acid, succinic acid, glutaric acid, malic acid, tartaric acid, maleic acid, fumaric acid, saccharic acid and carboxymethyl inulines, monomeric and polymeric aminopolycarboxylic acids, in particular glycine diacetic acid, methylglycine diacetic acid, nitrilotriacetic acid (NTA), iminodisuccinates such as ethylenediamine-N,N′-disuccinic acid and hydroxyiminodisuccinates, ethylenediaminetetraacetic acid and polyaspartic acid, polyphosphonic acids, in particular aminotris(methylenephosphonic acid), ethylenediamine tetrakis(methylenephosphonic acid), lysine tetra(methylenephosphonic acid) and 1-hydroxyethane-1,1-diphosphonic acid, polymeric hydroxy compounds such as dextrin, and polymeric (poly)carboxylic acids, polycarboxylates which can be obtained in particular by oxidizing polysaccharides or dextrins, and/or polymeric acrylic acids, methacrylic acids, maleic acids, and mixed polymers thereof, which may also contain small amounts of polymerizable substances without carboxylic acid functionality in polymerized form. Organic builder substances of this kind can, if desired, be contained in amounts of up to 50 wt. %, in particular up to 25 wt. %, preferably of from 10 to 20 wt. % and particularly preferably from 1 to 5 wt. %. In addition to their builder effect, the free acids typically also have the property of being an acidification component and are thus also used for setting a lower and milder pH of agents. Particularly noteworthy here are citric acid, succinic acid, glutaric acid, adipic acid, gluconic acid, and any mixtures thereof. Citric acid or salts of citric acid are particularly preferably used as builder substances. Further particularly preferred builder substances are selected from methylglycinediacetic acid (MGDA), glutamic acid diacetate (GLDA), aspartic acid diacetate (ASDA), hydroxyethyl iminodiacetate (HEIDA), iminodisuccinate (IDS), ethylenediamine disuccinate (EDDS), carboxymethyl inulin and polyaspartate. In preferred embodiments, citric acid and/or citrate is used as the water-soluble, organic builder. It is particularly preferred to use 0.5 to 25 wt. %, preferably 0.75 to 12.5 wt. %, more preferably 1 to 4 wt. %, citric acid and/or 0.5 to 25 wt. %, preferably 0.75 to 12.5 wt. %, more preferably 1 to 4 wt. %, citrate, preferably alkali citrate, more preferably sodium citrate. Citric acid/citrate can each be used in the form of their hydrates, for example citric acid can be used in the form of the monohydrate, and citrate can be used in the form of the trisodium citrate dihydrate.

Polymeric polycarboxylates are also suitable as builders, e.g., the alkali metal salts of polyacrylic acid or polymethacrylic acid, e.g., those with a relative molecular mass of 500 to 70000 g/mol. For the purpose of this application, the molar masses indicated for polymeric polycarboxylates are weight-average molar masses Mw of the respective acid form which have been determined in principle using gel permeation chromatography (GPC), a UV detector having been used. The measurement was carried out against an external polyacrylic acid standard which, due to the structural relationship to the tested polymers, yields realistic molecular weight values. These specifications differ significantly from the molecular weight specifications for which polystyrene sulfonic acids are used as the standard. The molar masses measured against polystyrene sulfonic acids are generally considerably higher than the molar masses indicated in this application. Suitable polymers are in particular polyacrylates which preferably have a molecular mass of from 2000 to 20000 g/mol. Due to their superior solubility, short-chain polyacrylates having molar masses of from 2000 to 10000 g/mol, and particularly preferably of from 3000 to 5,000 g/mol, may in turn be preferred from this group. In addition, copolymeric polycarboxylates are suitable, in particular those of acrylic acid with methacrylic acid and those of acrylic acid or methacrylic acid with maleic acid. Copolymers of acrylic acid with maleic acid containing 50 to 90 wt. % acrylic acid and 50 to 10 wt. % maleic acid have proven to be particularly suitable. The relative molecular mass thereof, based on free acids, is generally 2000 to 70000 g/mol, preferably 20000 to 50000 g/mol, and in particular 30000 to 40000 g/mol.

A solid agent according to the invention preferably contains at least one water-soluble and/or water-insoluble, organic and/or inorganic builder. The water-soluble organic builder substances include the aforementioned organic builder substances.

In addition to the water-soluble organic builders mentioned above, the agents of the invention may also further contain inorganic water-soluble builders. In particular, alkali silicates, alkali carbonates, alkali hydrogen carbonates, alkali phosphates and/or sesquicarbonates, which can be present in the form of their alkaline, neutral, or acidic sodium or potassium salts, can be used as water-soluble inorganic builder materials. Small amounts of calcium carbonate may optionally also be contained in solid textile washing agents. Water-soluble crystalline and/or amorphous alkali silicates are suitable, for example. The alkali silicates that can be used as builders in the agents according to the invention preferably have a molar ratio of alkali oxide to SiO₂ of less than 0.95, in particular from 1:1.1 to 1:12, and may be present in amorphous or crystalline form. Preferred alkali silicates are sodium silicates, in particular amorphous sodium silicates, having a molar ratio of Na₂O:SiO₂ of from 1:2 to 1:2.8. As crystalline silicates, which may be present alone or in admixture with amorphous silicates, crystalline phyllosilicates of the general formula Na₂SiₓO₂ₓ₊₁·y H₂O are preferably used, in which x, known as the modulus, is a number from 1.9 to 22, in particular 1.9 to 4, and y is a number from 0 to 33, and preferred values for x are 2, 3 or 4. Preferred crystalline phyllosilicates are those in which x assumes the values 2 or 3 in the mentioned general formula. In particular, both β- and δ-sodium disilicates (Na₂Si₂O₅·y H₂O) are preferred. Practically water-free crystalline alkali silicates of the above general formula, in which x is a number from 1.9 to 2.1 and which are produced from amorphous alkali silicates, may also be used in agents according to the invention. In a further embodiment of agents according to the invention, a crystalline sodium phyllosilicate having a module of from 2 to 3, as can be prepared from sand and soda, is used. Crystalline sodium silicates having a module in the range of from 1.9 to 3.5 are used in a further embodiment of agents according to the invention. In agents containing amorphous and crystalline alkali silicates, the weight ratio of amorphous alkali silicate to crystalline alkali silicate is preferably from 1:2 to 2:1 and in particular from 1:1 to 2:1. Crystalline phyllosilicates of the above formula (I) are sold by Clariant GmbH under the trade name Na-SKS, for example Na-SKS-1 (Na₂Si₂₂O₄₅·x H₂O, kenyaite), Na-SKS-2 (Na₂Si₁₄O₂₉·x H₂O, magadiite), Na-SKS-3 (Na₂Si₈O₁₇·x H₂O) or Na-SKS-4 (Na₂Si₄O₉·x H₂O, makatite). Of these, Na-SKS-5 (α-Na₂Si₂O₅), Na-SKS-7 (B-Na₂Si₂O₅, natrosilite), Na-SKS-9 (NaHSi₂O₅·3 H₂O), Na-SKS-10 (NaHSi₂O₅·3 H₂O, kanemite), Na-SKS-11 (t-Na₂Si₂O₅) and Na-SKS-13 (NaHSi₂O₅), but in particular Na-SKS-6 (δ-Na₂Si₂O₅), are particularly suitable. In one embodiment of agents according to the invention, a granular compound made of crystalline phyllosilicate and citrate, crystalline phyllosilicate and the above-described (co)polymeric polycarboxylic acid, or alkali silicate and alkali carbonate is used, as is commercially available under the name Nabion® 15, for example. Water-soluble inorganic builder materials of this kind are contained in agents according to the invention preferably in amounts of from 1 to 20 wt. %, in particular 5 to 15 wt. %. Also of significance as water-soluble inorganic builder substances are the carbonates (and hydrogen carbonates), in particular sodium carbonate, and the phosphonic acids/phosphonates.

The agents according to the invention are preferably free of phosphate builders, i.e., they contain less than 1 wt. %, and preferably no phosphate builders are added intentionally.

The agents can also contain water-insoluble builders. Crystalline or amorphous water-dispersible alkali aluminosilicates, in amounts of up to 50 wt. %, preferably not more than 40 wt. %, in particular of from 3 to 20 wt. % and particularly preferably from 1 to 15 wt. %, are used in particular as water-insoluble inorganic builder materials. Among these, the crystalline sodium aluminosilicates in washing agent quality, in particular zeolite A, zeolite P, zeolite MAP and optionally zeolite X, either alone or in mixtures, for example in the form of a co-crystallizate of the zeolites A and X (Vegobond® AX, a commercial product from Condea Augusta S.p.A.), are preferred. Amounts close to the stated upper limit are preferably used in solid, particulate agents. Suitable aluminosilicates have, in particular, no particles having a particle size above 30 μm and preferably consist by at least 80 wt. % of particles having a size below 10 μm. The calcium binding capacity, which can be determined according to DE 2412837 A1, of said aluminosilicates is generally in the range of from 100 to 200 mg CaO per gram.

In addition to the builders described above, the agent may contain cleaning-active polymers. The proportion by weight of the cleaning-active polymers with respect to the total weight of agents according to the invention is preferably 0.1 to 20 wt. %, more preferably 1.0 to 15 wt. %, and even more preferably 2.0 to 12 wt. %.

Possible peroxygen compounds suitable for use in the agents according to the invention include, in particular, organic peroxy acids or peracid salts of organic acids, such as phthalimidopercaproic acid, perbenzoic acid, or salts of diperdodecanedioic acid, hydrogen peroxide and inorganic salts releasing hydrogen peroxide under the washing conditions, which salts include perborate, percarbonate, persilicate, and/or persulfates such as caroate, as well as hydrogen peroxide inclusion compounds such as H₂O₂-urea adducts. Hydrogen peroxide can also be produced by means of an enzymatic system, i.e., an oxidase and the substrate thereof. If solid peroxygen compounds are intended to be used, these may be used in the form of powders or granules, which may also be coated in a manner known in principle. The peroxygen compounds can be added to the washing liquor as such or in the form of the agents containing them, which in principle can contain all conventional washing, cleaning or disinfectant components. Particularly preferably, alkali percarbonate or alkali perborate monohydrate is used. If an agent according to the invention contains peroxygen compounds, these are present in amounts of preferably up to 50 wt. %, in particular of 5 to 30 wt. %, and more preferably of 0.1 to 20 wt. %.

Compounds which, under perhydrolysis conditions, result in aliphatic peroxocarboxylic acids having preferably 1 to 10 C atoms, in particular 2 to 4 C atoms, and/or optionally substituted perbenzoic acid, may be used in the agents as bleach activators. Suitable substances are those which carry O- and/or N-acyl groups of the stated number of C atoms and/or optionally substituted benzoyl groups. Preferred are polyacylated alkylene diamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates or carboxylates or the sulfonic or carboxylic acids thereof, in particular nonanoyloxybenzenesulfonate or isononanoyloxybenzenesulfonate or laroyloxybenzenesulfonate (NOBS or iso-NOBS or LOBS), 4-(2-decanoyloxyethoxycarbonyloxy)-benzenesulfonate (DECOBS) or decanoyloxybenzoate (DOBA), carboxylic acid anhydrides, in particular phthalic acid anhydride, acylated polyhydric alcohols, in particular triacetin, ethylene glycol diacetate, 2,5-diacetoxy-2,5-dihydrofuran and enol esters, as well as acetylated sorbitol and mannitol or the described mixtures thereof (SORMAN), acylated sugar derivatives, in particular pentaacetyl glucose (PAG), pentaacetyl fructose, tetraacetylxylose and octaacetyl lactose, acetylated, optionally N-alkylated glucamine and gluconolactone, N-acylated lactams, for example N-benzoylcaprolactam, nitriles from which perimidic acids are formed, in particular aminoacetonitrile derivatives having a quaternized nitrogen atom, and/or oxygen-transferring sulfonimines and/or acylhydrazones. The hydrophilically substituted acyl acetals and the acyl lactams are likewise preferably used. Combinations of conventional bleach activators can also be used. Such bleach activators can, in particular in the presence of the above-mentioned hydrogen peroxide-yielding bleaching agents, be present in the customary quantity range, preferably in amounts of from 0.5 to 10 wt. %, and in particular 1 to 8 wt. %, based on the total agent, but are preferably entirely absent when percarboxylic acid is used as the sole bleaching agent.

In addition to or instead of the conventional bleach activators, sulfonimines and/or bleach-boosting transition metal salts or transition metal complexes may also be contained in solid agents as what are referred to as bleach catalysts.

Suitable graying inhibitors or soil release active ingredients (soil release polymer) are cellulose ethers, such as carboxymethyl cellulose, methyl cellulose, hydroxyalkyl celluloses and mixed cellulose ethers, such as methyl hydroxyethyl cellulose, methylhydroxypropyl cellulose and methyl carboxymethyl cellulose. Sodium carboxymethyl cellulose, hydroxypropyl methyl cellulose and mixtures thereof and, optionally, mixtures thereof with methyl cellulose are preferably used. The soil release active ingredients commonly used include copolyesters containing dicarboxylic acid units, alkylene glycol units and polyalkylene glycol units. The proportion of graying inhibitors and/or soil release active ingredients in agents according to the invention is generally no greater than 2 wt. % and is preferably 0.5 to 1.5 wt. %, particularly preferably 0.5 to 2 wt. %.

Derivatives of diaminostilbene disulfonic acid or the alkali metal salts thereof can be contained, for example, as optical brighteners, in particular for textiles made of cellulose fibers (e.g., cotton). Salts of 4,4′-bis(2-anilino-4-morpholino-1,3,5-triazinyl-6-amino)stilbene-2,2′-disulfonic acid or compounds having a similar structure which, instead of the morpholino group, have a diethanolamino group, a methylamino group, or a 2-methoxyethylamino group are suitable, for example. Furthermore, brighteners of the substituted 4,4′-distyryl-diphenyl type can be present, e.g., 4,4′-bis-(4-chloro-3-sulfostyryl)-diphenyl. Mixtures of brighteners can also be used. Brighteners of the 1,3-diaryl-2-pyrazoline type, e.g., 1-(p-sulfoamoylphenyl)-3-(p-chlorophenyl)-2-pyrazoline, and compounds having a similar structure are particularly suitable for polyamide fibers. The content of optical brighteners or brightener mixtures in the agent is generally no greater than 1 wt. %, preferably 0.05 to 0.5 wt. %. In a preferred embodiment of the invention, the agent is free of such active ingredients.

The customary foam regulators that can be used in the agents according to the invention include, for example, polysiloxane-silicic acid mixtures, the finely divided silicic acid contained therein preferably being silanized or otherwise hydrophobized. The polysiloxanes can consist of both linear compounds and crosslinked polysiloxane resins and mixtures thereof. Further defoamers are paraffinic hydrocarbons, in particular microparaffins and paraffin waxes of which the melting point is above 40° C., saturated fatty acids or soaps having in particular 20 to 22 C atoms, for example sodium behenate, and alkali salts of phosphoric acid mono- and/or dialkyl esters, in which the alkyl chains each have 12 to 22 C atoms. Among these, sodium monoalkyl phosphate and/or dialkyl phosphate having C₁₆₋₁₈ alkyl groups is preferably used. The proportion of foam regulators can preferably be 0.2 to 2 wt. %, particularly preferably not more than 1 wt. %.

In order to set the desired pH, the agents according to the invention can contain acids that are compatible with the system and environment, in particular citric acid, acetic acid, tartaric acid, malic acid, lactic acid, glycolic acid, succinic acid, glutaric acid, and/or adipic acid, but also mineral acids, in particular sulfuric acid or alkali hydrogen sulfates, or bases, in particular ammonium or alkali hydroxides, preferably sodium hydroxide. These types of pH regulators are contained in the agents according to the invention in amounts preferably no greater than 10 wt. %, in particular of from 0.5 to 6 wt. %, particularly preferably from 0.3 to 2 wt. %.

The cleaning agents according to the invention can contain an organic solvent as a further component. Adding organic solvents has an advantageous effect on the enzyme stability and cleaning performance of these agents. Preferred organic solvents are derived from the group of monohydric or polyhydric alcohols, alkanolamines or glycol ethers.

The solvents are preferably selected from ethanol, n- or i-propanol, butanol, glycol, propanediol, butanediol, glycerol, diglycol, propylene diglycol, butyl diglycol, hexylene glycol, ethylene glycol methyl ether, ethylene glycol ethyl ether, ethylene glycol propyl ether, ethylene glycol mono-n-butyl ether, diethylene glycol methyl ether, diethylene glycol ethyl ether, propylene glycol methyl ether, propylene glycol ethyl ether, propylene glycol propyl ether, dipropylene glycol methyl ether, dipropylene glycol ethyl ether, methoxytriglycol, ethoxytriglycol, butoxytriglycol, 1-butoxyethoxy-2-propanol, 3-methyl-3-methoxybutanol, propylene-glycol-t-butyl ether and mixtures of these solvents. The proportion by weight of these organic solvents with respect to the total weight of the agents according to the invention is preferably 0.1 to 10 wt. %, more preferably 0.2 to 8.0 wt. %, and even more preferably 0.5 to 5.0 wt. %. A particularly preferred organic solvent which is particularly effective in stabilizing the agents is glycerol, as well as 1,2-propylene glycol. Liquid agents preferably comprise at least one polyol, preferably from the group glycerol and 1,2-propylene glycol, based on the total weight of the agent, preferably in an amount of from 0.1 to 10 wt. %, preferably 0.2 to 8.0 wt. %, and more preferably 0.5 to 5.0 wt. %. Other preferred organic solvents are the organic amines and alkanolamines. The agents according to the invention preferably contain these amines in amounts of 0.1 to 10 wt. %, more preferably of 0.2 to 8.0 wt. %, and even more preferably of 0.5 to 5.0 wt. %, in each case based on the total weight thereof. Ethanolamine is a particularly preferred alkanolamine.

Washing and/or cleaning agents according to the invention may exclusively contain the enzymes according to the invention. Alternatively, they can also contain further hydrolytic enzymes or other enzymes in a concentration expedient for the effectiveness of the agent. A further embodiment of the invention is thus represented by agents that further comprise one or more further enzymes. All enzymes which can develop catalytic activity in the agent according to the invention, in particular a protease, lipase, amylase, cellulase, hemicellulase, mannanase, tannanase, xylanase, xanthanase, xyloglucanase, B-glucosidase, pectinase, carrageenanase, perhydrolase, oxidase, oxidoreductase, and mixtures thereof, can preferably be used as further enzymes. Further enzymes are advantageously contained in the agent in each case in an amount of 1×10⁻⁸ to 5 wt. %, based on the active protein. Increasingly preferably, each further enzyme is contained in agents according to the invention in an amount of from 1×10⁻⁷ to 3 wt. %, from 0.00001 to 1 wt. %, from 0.00005 to 0.5 wt. %, from 0.0001 to 0.1 wt. % and particularly preferably from 0.0001 to 0.05 wt. %, based on active protein. Particularly preferably, the enzymes exhibit synergistic cleaning performance with respect to particular dirt or stains, i.e., the enzymes contained in the agent composition assist one another in their cleaning performance.

Examples of proteases are the subtilisins BPN′ from Bacillus amyloliquefaciens and Carlsberg from Bacillus licheniformis, protease PB92, subtilisins 147 and 309, the protease from Bacillus lentus, subtilisin DY, and the enzymes thermitase, proteinase K and proteases TW3 and TW7, which in the narrower sense are associated with the subtilases but no longer with the subtilisins. Subtilisin Carlsberg is available in a developed form under the trade name Alcalase® from Novozymes. Subtilisins 147 and 309 are marketed by Novozymes under the trade names Esperase® and Savinase®, respectively. Protease variants, described, for example, in WO 95/23221, WO 92/21760 WO 2013/060621 and EP 3660151 are derived from the protease from Bacillus lentus DSM 5483. Other proteases that are suitable are, for example, the enzymes available under the trade names Durazym®, Relase®, Everlase®, Nafizym®, Natalase®, Kannase®, Progress Uno 101L® and Ovozyme® from Novozymes, the enzymes available under the trade names Purafect®, Purafect® OxP, Purafect® Prime, Excellase® and Properase®, Preferenz P100® and Preferenz P300® from Danisco/DuPont, the enzyme available under the trade name Lavergy pro 104 LS® from BASF, the enzyme available under the trade name Protosol® from Advanced Biochemicals Ltd., the enzyme available under the trade name Wuxi® from Wuxi Snyder Bioproducts Ltd., the enzymes available under the trade names Proleather® and Protease P® from Amano Pharmaceuticals Ltd., and the enzyme available under the name Proteinase K-16 from Kao Corp. The proteases from Bacillus gibsonii and Bacillus pumilus, which are disclosed in WO 2008/086916, WO 2007/131656, WO 2017/215925, WO 2021/175696 and WO 2021/175697, are particularly preferably used.

Examples of amylases are α-amylases from Bacillus licheniformis, Bacillus amyloliquefaciens or Bacillus stearothermophilus, as well as in particular the developments thereof that have been improved for use in washing and/or cleaning agents. The enzyme from Bacillus licheniformis is available from Novozymes under the name Termamyl® and from Danisco/DuPont under the name Purastar® ST. Development products of this α-amylase are available under the trade names Duramyl® and Termamyl® ultra (both from Novozymes), Purastar® OxAm (Danisco/DuPont) and Keistase® (Daiwa Seiko Inc.). The α-amylase from Bacillus amyloliquefaciens is marketed by Novozymes under the name BAN®, and derived variants from the α-amylase from Bacillus stearothermophilus are marketed under the names BSG® and Novamyl®, also by Novozymes. Furthermore, for this purpose the α-amylases from Bacillus sp. A 7-7 (DSM 12368) and the cyclodextrin glucanotransferase (CGTase) from Bacillus agaradherens (DSM 9948) should be emphasized. Fusion products of all mentioned molecules can also be used. Furthermore, the developments of the α-amylase from Aspergillus niger and A. oryzae, available under the trade name Fungamyl® from Novozymes, are suitable. Other commercial products that can be advantageously used are, for example, Amylase-LT® and Stainzyme® or Stainzyme® ultra or Stainzyme® plus as well as Amplify™ 12 L or Amplify Prime™ 100 L, the latter also from Novozymes, and the PREFERENZ S® series from Danisco/DuPont, including, for example, PREFERENCE S100®, PREFERENCE S1000® or PREFERENCE S210®. Variants of these enzymes that can be obtained by point mutations may also be used according to the invention.

Suitable cellulases include those of bacterial or fungal origin. Chemically modified or protein-engineered mutants are included. Suitable cellulases are cellulases from the genera Bacillus, Pseudomonas, Humicola, Fusarium, Thielvia, Acremonium, e.g., the fungal cellulase from Humicola insolens, Mycelophthora thermophila and Fusarium oxysporum, which are disclosed in U.S. Pat. Nos. 4,435,307, 5,648,263, 5,691,178, 5,776,757 and WO 89/09259. Particularly suitable cellulases are the alkaline or neutral cellulases with color care properties. Examples of such cellulases are cellulases which are described in EP 0495257, EP 0531372, WO 96/11262, WO 96/29397 and WO 98/08940. Other examples are cellulase variants as described in WO 94/07998, EP 0531315, EP 3212777, EP 3502243, EP 3653705, EP 3653706, U.S. Pat. Nos. 5,457,046, 5,686,593, 5,763,254, WO 95/24471, WO 98/12307 and WO 99/01544 and WO 2019/122520. Examples of cellulases with endo-1,4-glucanase activity (EC 3.2.1.4) are described in WO 2002/099091, for example those having a sequence of at least 97% identity to the amino acid sequence of positions 1 to 773 of SEQ ID NO:2 of WO 2002/099091. A further example can comprise a GH44-xyloglucanase, for example a xyloglucanase enzyme having a sequence of at least 60% identity to positions 40 to 559 of SEQ ID NO:2 of WO 2001/062903. Other examples of cellulases comprise the GH45 cellulases described in WO 96/29397. Commercially available cellulases include Celluzyme™, Carezyme™, Carezyme Premium™, Celluclean™ (e.g., Celluclean™ 5000 L and Celluclean™ 4000T), Celluclean Classic™, Cellusoft™, Endolase®, Renozyme® and Whitezyme™ (Novozymes A/S), Clazinase™ and Puradax HA™ (Genencor International Inc.), KAC-500(B)™ (Kao Corporation), Revitalenz™ 1000, Revitalenz™ 2000 and Revitalenz™ 3000 (DuPont), as well as Ecostone® and Biotouch® (AB Enzymes).

Suitable lipases are, for example, from Thermomyces, e.g., from T. lanuginosus (formerly Humicola lanuginosa) as described in EP 0258068 and EP 0305216, lipase from strains of Pseudomonas (some of them now renamed Burkholderia), e.g., P. alcaligenes or P. pseudoalcaligenes, P. cepacia, P. sp. strain SD705, P. wisconsinensis, Streptomyces lipases of the GDSL type, lipase from Thermobifida fusca, lipase from Geobacillus stearothermophilus, lipase from Bacillus subtilis and lipase from Streptomyces griseus and S. pristinaespiralis. The lipases that can originally be obtained from Humicola lanuginosa (Thermomyces lanuginosus) or have been developed therefrom, in particular those having one or more of the following amino acid exchanges in positions D96L, T213R and/or N233R, particularly preferably T213R and N233R, proceeding from the mentioned lipase, belong to the preferred lipases. Preferred commercial lipase products include Lipolase™, Lipex™, Lipolex™, and Lipoclean™ (Novozymes A/S), Lumafast (Genencor/DuPont), and Lipomax (Gist-Brocades).

Suitable mannanases are, for example, Bacillus subtilis endo-β-mannanase, Bacillus sp. 1633 endo-β-mannanase, Bacillus sp. AAI12 endo-β-mannanase, Bacillus sp. AA349 endo-β-mannanase, Bacillus agaradhaerens NCIMB 40482 endo-β-mannanase, Bacillus halodurans endo-β-mannanase, Bacillus clausii endo-β-mannanase, Bacillus licheniformis endo-β-mannanase, Humicola insolens endo-β-mannanase and Caldocellulosiruptor sp. endo-β-mannanase (cf. e.g. U.S. Pat. No. 6,060,299, WO 99/64573, U.S. Pat. No. 6,566,114 and WO 99/64619).

Pectate lyases suitable for washing and cleaning agents are described, for example, in WO 2003/095638 or WO 2015/121133. Examples of suitable pectinolytic enzymes are also the enzymes and enzyme preparations available under the trade names Gamanase®, Pektinex AR®, X-Pect® or Pectaway® from Novozymes, under the trade names Rohapect UF®, Rohapect TPL®, Rohapect PTE100®, Rohapect MPE®, Rohapect MA plus HC, Rohapect DA12L®, Rohapect 10L®, Rohapect B1L® from AB Enzymes, and under the trade name Pyrolase® from Diversa Corp.

In the cleaning agents described herein, the enzymes to be used can further be formulated together with accompanying substances, for example from fermentation. In liquid formulations, the enzymes are preferably used as liquid enzyme formulation(s).

The enzymes are generally not provided in the form of the pure protein, but rather in the form of stabilized, storable and transportable preparations. These ready-made preparations include, for example, the solid preparations obtained by means of granulation, extrusion or lyophilization or, in particular in the case of liquid or gel agents, solutions of the enzymes, which are advantageously as concentrated as possible, have a low water content, and/or are admixed with stabilizers or further auxiliaries.

Alternatively, the enzymes can be encapsulated both for the solid and for the liquid administration form, for example by means of spray-drying or extrusion of the enzyme solution together with a preferably natural polymer or in the form of capsules, for example those in which the enzymes are enclosed as in a solidified gel, or in those of the core-shell type, in which an enzyme-containing core is coated with a protective layer that is impermeable to water, air and/or chemicals. Further active ingredients, for example, stabilizers, emulsifiers, pigments, bleaches or dyes can additionally be applied in overlaid layers. Such capsules are made using methods that are known per se, for example by means of vibratory granulation or roll granulation or by means of fluid bed processes. Advantageously, such granules are low in dust, for example due to the application of polymeric film formers, and are stable in storage due to the coating.

Furthermore, it is possible to formulate two or more enzymes together such that a single granule exhibits a plurality of enzyme activities.

The enzymes can also be introduced into water-soluble films, such as those used in the formulation of washing and cleaning agents in a unit dosage form. Such a film allows the enzymes to be released after contact with water. As used herein, “water-soluble” refers to a film structure that is preferably completely water-soluble. Preferably, such a film consists of (completely or partially hydrolyzed) polyvinyl alcohol (PVA).

The agents according to the invention may comprise one or more reversible enzyme inhibitor(s)/stabilizer(s). The agents according to the invention may contain the reversible enzyme inhibitor(s)/stabilizer(s) in a concentration of 0.1 to 2 wt. %, preferably 0.3 to 1.5 wt. %, based on the total weight of the agent. If several inhibitors/stabilizers are contained, this information refers to the total concentration.

These may in particular be selected from the group consisting of polyols, such as glycerol or 1,2-ethylene glycol, benzamidine hydrochloride, borax, boric acids, boronic acids or their salts or esters or derivatives, in particular phenylboronic acid derivatives or 4-formylphenylboronic acid (4-FPBA), antioxidants, special peptide compounds and combinations thereof.

In the context of the present invention, “phenylboronic acid derivative” is understood to mean a compound of formula (III). The compound of formula (III) has the following structural formula:

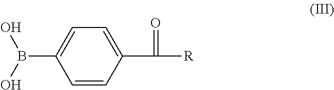

where R is hydrogen, a hydroxyl group, a C₁₋₆ alkyl group, a substituted C₁₋₆ alkyl group, a C₁₋₆ alkenyl, or a substituted C₁₋₆ alkenyl group. Preferably, the functional group R in the phenylboronic acid derivative is a C₁₋₆ alkyl group and, among these, is more preferably —CH₃, —CH₃CH₂, or —CH₃CH₂CH₂. More preferably, the functional group R in the phenylboronic acid derivative is hydrogen. Particularly preferably, the phenylboronic acid derivative is 4-formylphenylboronic acid (4-FPBA).

The inhibitor/stabilizer compound used can be boric acid.

In particularly preferred embodiments, the washing and/or cleaning agent according to the invention is substantially free of boron-containing compounds. “Substantially free from boron-containing compounds” in this context means that the agents according to the invention contain less than 2 wt. %, preferably less than 1 wt. %, more preferably less than 0.5 wt. %, and particularly preferably less than 0.1 wt. %, of boron-containing compounds, based on the total weight of the agent. In very particularly preferred embodiments, the agents according to the invention are free of boron-containing compounds, i.e. in particular they do not contain any boric acid and/or phenylboronic acid derivatives.

In various embodiments, the enzyme and the inhibitor/stabilizer compound may be pre-formulated in an enzyme composition. As is clear from the preceding remarks, the enzyme protein forms only a fraction of the total weight of conventional enzyme preparations. Enzyme preparations that are preferably used contain between 0.1 and 40 wt. %, preferably between 0.2 and 30 wt. %, particularly preferably between 0.4 and 20 wt. %, and in particular between 0.8 and 10 wt. % of the enzyme protein. In such compositions, the inhibitor/stabilizer compound can be contained in an amount of from 0.05 to 35 wt. %, preferably from 0.05 to 10 wt. %, based on the total weight in the enzyme composition. This enzyme composition, which is also a constituent of the present invention, can then be used in agents according to the invention, specifically in amounts which lead to the final concentrations in the agent which are indicated above.

A further object of the invention is a method for cleaning textiles and/or hard surfaces, in particular dishes, which is characterized in that in at least one method step, an agent according to the invention is used.

In various embodiments, the method described above is characterized in that it is carried out at a temperature of from 0° C. to approximately 100° C., preferably from approximately 20° C. to approximately 60° C., more preferably from approximately 20° C. to approximately 40° C., particularly preferably of approximately 30° C.

This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent. This includes both manual and machine methods, with machine methods being preferred because they can be controlled more precisely, for example with regard to the quantities used and contact times. Methods for cleaning textiles are generally characterized by the fact that, in a plurality of method steps, various cleaning-active substances are applied to the material to be cleaned and washed off after the exposure time, or in that the material to be cleaned is otherwise treated with a washing agent or a solution or dilution of this agent. This also comprises methods using (semi-)automated washing or cleaning systems such as floor-mopping robots or wet vacuum cleaners.

This object of the invention also comprises a dishwashing machine method. The cleaning agent according to the invention can be dispensed into the cleaning liquor in such a method for example by means of the dispensing chamber in the door or by means of an additional dispensing container in the interior of the dishwasher. Alternatively, the cleaning agent can also be applied directly to the dirty dishes or to one of the interior walls of the dishwasher, for example the inside of the door. The method according to the invention is carried out in the interior of a commercially available dishwasher. In the case of a dishwasher, the cleaning program can generally be selected and determined by the user before the dishwashing method is carried out. The dishwasher cleaning program used in the method according to the invention comprises at least one prewash cycle and one cleaning cycle. Cleaning programs which comprise further cleaning or rinsing cycles, e.g., a rinse cycle, are preferred according to the invention. The method according to the invention is particularly preferably part of a cleaning program comprising a prewash cycle, a cleaning cycle and a rinse cycle. The method according to the invention is preferably used in connection with cleaning programs in which the washing liquor is heated during the cleaning cycle. In a preferred embodiment of the method according to the invention, the cleaning cycle during which the cleaning agent according to the invention is dispensed into the interior of the dishwasher is characterized in that the temperature of the cleaning liquor during said cycle rises to values above 30° C., preferably above 40° C. and in particular above 50° C.

Alternative embodiments of this subject matter of the invention are also represented by methods for treating textile raw materials or for textile care, in which an agent according to the invention becomes active in at least one method step. Among these, methods for textile raw materials, fibers or textiles comprising natural constituents are preferred, and very particularly for those comprising wool or silk.

A further subject matter of the invention is the use of an agent according to the invention for cleaning textiles and/or hard surfaces, in particular dishes, in particular for deep cleaning, preferably in a temperature range of approximately 20° C. to approximately 60° C., approximately 40° C., particularly preferably approximately 30° C.

A further subject matter of the invention is the use of a dispersin in a washing or cleaning agent according to the invention to improve the cleaning performance of the agent on various stains, preferably in a temperature range of approximately 20° C. to approximately 60° C., preferably approximately 40° C., particularly preferably approximately 30° C.

A further subject matter of the invention is the use of a dispersin in a washing and/or cleaning agent according to the invention for reducing the malodor of a textile or a surface that has been treated with the agent, as described in Example 1, preferably in a temperature range of approximately 20° C. to approximately 60° C., preferably 40° C., particularly preferably approximately 30° C. One embodiment of the invention relates to the use of an agent containing a dispersin and one or more fragrances for reducing malodor. One embodiment of the invention relates to the use of an agent as defined herein comprising a dispersin and one or more fragrances for reducing malodor. A preferred embodiment of the invention relates to the use of a washing and/or cleaning agent as defined herein, comprising a dispersin and one or more fragrances for reducing malodor when the agent is used, for example, in a textile washing process. A preferred embodiment of the invention relates to the use of a washing and/or cleaning agent as defined herein, comprising a dispersin and one or more fragrances for reducing malodor on an article, e.g., a textile.

A further subject matter of the invention is the use of a dispersin in a washing and/or cleaning agent according to the invention for increasing the fragrance intensity of the fragrance contained in the agent on a textile or a surface which has been treated with the agent, when this is determined as described in Example 1, preferably in a temperature range of approximately 20° C. to approximately 60° C., preferably 40° C., particularly preferably approximately 30° C.

One embodiment of the invention relates to the use of a washing and/or cleaning agent, as defined herein, comprising a dispersin and one or more fragrances for reducing malodor, wherein the dispersin exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion.

One embodiment of the invention relates to the use of a washing and/or cleaning agent as defined herein, comprising a dispersin and one or more fragrances for reducing malodor, wherein the dispersin exhibits β-1,6-N-acetylglucosaminidase activity, comprises an amino acid sequence identical to the amino acid sequence indicated in SEQ ID NO:1 over its entire length by at least 70% and increasingly preferably by at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, and, based on the numbering according to SEQ ID NO:1, at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323, has at least one amino acid substitution and/or deletion selected from the group consisting of D2A, D2L, D2N, D2R, D2V, D2W, Q3F, Q3I, Q3L, Q3M, Q3P, Q3V, Q3Y, Q3T, S12A, H15F, H15Y, T17W, T17C, T17E, T17F, T17M, T17R, T17V, V18L, E19D, E19N, E19P, K22A, K22M, K22V, S23C, S23E, S23I, S23L, S23R, S23T, S23V, V25R, D26M, Y30*, Y30D, Y30L, Y30M, Y30N, Y30R, Y30T, Y30V, G32L, G32M, G32R, N43*, N43H, N43L, E44*, N45D, N45L, N45V, A49W, A49Y, Y52*, Y52M, G54L, G54M, G54N, S56T, S56W, S57W, E58N, N59A, N59C, N59D, N59E, N59F, N59M, N59R, N59V, N59W, T60V, N62C, N62D, N62H, N62Q, N62W, T63C, T63D, T63L, T63N, T63R, T63V, K67A, K67L, N68L, N68Q, L71H, L71N, L71R, L71V, L71W, S72*, S72C, S72D, S72E, S72F, S72G, S72I, S72M, S72N, S72R, S72T, S72Y, I74L, S77A, D79V, K80*, K80E, K80H, K80L, K80N, K80Q, K80V, K80W, D81A, D81G, D81L, D81R, D81S, D81T, D81V, D81W, I82V, L90F, E99Q, E99R, L100S, K103A, K103R, K104N, K104W, D105N, V106A, V106D, V106E, V106H, V106K, V106L, V106M, V106N, V106Q, V106W, V106Y, K107A, K107C, K107L, K107M, K107T, K107V, K107W, N110M, N110R, N110V, D111A, D111E, D111M, D111N, D111Q, D111R, D111V, D111W, V113T, T114C, T114S, Y116D, Y116N, Y116R, S117*, S117D, S117H, S117N, S117P, E118*, E118A, E118D, E118G, E118L, E119G, E119W, T120I, T120L, T120M, T120V, T120W, D122*, D122H, D122R, Y123W, Y124C, Y124I, Y124K, Y124L, Y124M, Y124Q, Y124R, Y124T, Y124V, Y124W, D125C, D125G, D125K, D125Q, D125R, N126V, R127D, R127H, R127K, R127L, R127M, R127Q, R127W, V128C, V128L, V128T, D131V, Q135*, Q135A, Q135D, Q135E, Q135K, Q135M, Q135Y, D138K, D138L, D138M, D138Q, D138R, D138S, D138V, D138W, E139W, D142R, D142W, Y145*, Y145H, Y145L, Y145N, Y145V, P147A, P147C, P147D, P147F, P147G, P147L, P147M, P147R, P147S, P147T, P147V, K148A, K148D, K148L, K148V, F149L, F149M, F149N, E150D, E150H, E150K, E150L, E150M, E150N, E150R, E150V, E150W, E150Y, G151A, G151C, G151D, G151L, G151N, G151P, G151S, G151W, K152D, K152L, K152R, G164D, G164E, G164H, G164S, G164V, V167D, V167E, V167L, V167P, V167Q, V167R, V167W, H168N, L170A, L170D, L170E, L170F, L170H, L170K, L170M, L170N, L170P, L170Q, L170R, L170S, L170V, L170W, L170Y, D171A, D171C, D171E, D171K, D171L, D171M, D171Q, D171R, D171V, D171W, D171Y, I173C, D174H, D174M, D174N, D174V, D174W, F175Y, N177M, Q178*, Q178A, Q178K, Q178R, Q178W, I179T, S181C, S181D, S181F, S181G, S181N, S181P, S181Q, S181T, S181V, S181W, E185M, E185R, E185V, E185W, S186D, S186E, S186H, S186I, S186K, S186L, S186M, S186N, S186Q, S186R, S186V, S186W, K187C, K187D, K187G, K187R, K187S, K187V, K187W, Y188P, E189L, E189V, E189W, S199C, S199L, S199M, S199Y, E200D, E200F, E200K, E200L, E200M, E200N, E200R, E200W, A203C, A203D, A203E, A203G, A203L, A203M, A203P, A203R, A203S, A203T, A203V, A203W, N204L, N204M, N204V, N204W, N204Y, L205I, D207A, D207C, D207E, D207G, D207N, D207Q, D207S, D207V, D207W, S208A, S208C, S208D, S208G, S208L, S208Q, S208T, S208V, S208W, S210T, Q215R, Q215M, Q215L, Q215*, S217V, T218A, T218L, T218Q, T218R, T218V, G222D, E224A, E224P, N227A, N227Q, N227R, N227S, N227T, N227K, D230*, D230R, D230T, D230W, E232D, E232V, N233H, N233Q, N233R, N233W, W234R, G235W, G235A, G235E, G235F, G235H, G235I, G235L, G235M, G235N, G235P, G235S, G235V, S237C, S237G, S237M, S237N, S237W, S237Y, Y244C, Y244E, Y244M, L249H, L249K, L249Q, L249R, L249W, L249Y, S251L, S251N, S251R, S251W, N252P, N252C, G253D, G253W, F254I, F254L, F254M, F254Y, Q256E, Q256R, N260*, N260A, N260C, N260E, N260I, N260K, N260L, N260M, N260Q, N260R, N260V, N260W, N260Y, E261*, E261A, E261D, E261R, E261W, Q262*, Q262F, Q262H, Q262W, Q262Y, M263K, M263L, M263Q, D264*, D264C, D264E, Y265F, N267S, N267T, W268C, W268E, W268M, W268R, Y270F, A271D, A271G, H272D, H272I, Y281P, Y282E, Y282N, Y281R, H272W, N273W, K274R, K274A, K274H, F276A, F276C, F276K, F276N, F276G, F276L, F276M, F276P, F276S, F276V, F276W, I278A, I278K, I278N, I278Q, I278V, S279C, S279D, S279E, S279G, S279N, D280C, D280E, Y281*, Y281A, Y281C, Y281H, Y281K, Y281N, Y281P, Y282E, Y282N, H283I, A284I, A284L, A284N, A284P, A284T, A284V, T287N, S288D, S288K, S288N, V290I, K291L, K291R, K291V, T296C, E300A, E300D, H301C, H301N, H301R, T303A, T303C, T303G, T303K, T303Q, T303R, T303W, D304C, D304M, L305M, L305N, S306C, K308A, K308D, K308G, K308I, K308L, K308Q, K308S, K308T, K308V, K308Y, K309A, K309C, K309D, K309H, K309L, K309M, K309N, K309Q, K309S, K309T, K309I, K312A, K312L, K312M, K312N, K312Q, K312S, K312W, E314I, E314L, E314V, L315I, L315V, R319A, N323R and combinations thereof, and wherein the one or more fragrances are selected from the group consisting of α-isomethyl ionone, benzyl salicylate, citronellol, coumarin, hexyl cinnamal, linalool, pentanoic acid, 2-methylethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-Iron, β-phenylethyl benzoate, α-santalol, cedrol, cedryl acetate, cedryl formate, cyclohexyl salicyate, γ-dodecalactone, p-phenylethylphenylacetate, 2-methyl-6-methylene-7-octen-2-ol (dihydroderivatives), 3A,4,5,6,7,7A-hexahydro-4,7-methano-1H-inden-5(or 6)-ylacetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-ylacetate, 3,7-dimethyl-3-octanol, 4-(2,6,6-trimethylcyclohex-1-en-1-yl)but-3-en-2-one, hedione, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allyl heptanoate, nerol, pin-2(3)-ene, diphenyl ether, methyl 2-naphthyl ether, methyl benzoate, 5-heptyldihydro-2(3H)-furanone, isopropyl ester myristic acid, γ-decalactone, ethyl 2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethyl phenyl ethyl acetal, b-4-dimethyl-3-cyclohexene-1-propanal, peppermint oil, d-p-mentha-1(6),8-dien-2-one and mixtures thereof.

All aspects, objects and embodiments described for the agent according to the invention are also applicable to these subjects of the invention. Therefore, reference is expressly made at this point to the disclosure at the corresponding point with the indication that this disclosure also applies to the above use according to the invention.

### EXAMPLES

### Example 1: Determination of Malodor and Fragrance Intensity

TABLE 1
WASHING AGENT MATRIX USED

| | Wt. % of active substance |
|---|---|
| Chemical name | in the formulation |
| Demineralized water | Remainder |
| Boric acid | 0.2 |
| Citric acid | 2.4 |
| FAEOS | 4.4 |
| FAEO | 9.2 |
| LAS | 16.8 |
| Palm kernel oil | 1.4 |
| NaOH | 0.3 |
| 1,2-propanediol | 9.9 |
| Glycerol | |
| HEDP | 0.4 |
| GLDA | 0.4 |
| Monoethanolamine | 5.7 |
| Misc. (enzymes (without dispersin), | minors |
| bitterns, opt. brighteners, perfume) | |

pH 8.0-8.6;
dosage 50 g/job

Dispersin and fragrance were incorporated into the washing agent matrix mentioned in Table 1 according to the scheme shown in Table 2. The dispersin was used with equal activity in the approaches to be compared.

TABLE 2

| | V1 | V2 | E1 |
|---|---|---|---|
| Dispersin (SEQ ID NO: 1 + Q3I-H15Y-A49W- | − | − | + |
| N59E-S163P-S186R-S225G-N227T-E232D- | | | |
| G235W-N252P-N260Q-H272V-S279D-Y281P- | | | |
| K308Q-K309E-K312Q) | | | |
| Fragrances | 0.75% | 0.6% | 0.6% |

In preparation, approximately 2.0 kg of white textiles (T-shirts, terry towels) and the household washing machine used (Miele Novotronic W1514) were cleaned and disinfected by repeated washing with a commercially available heavy-duty detergent containing bleach in the intensive program (95° C., 16° dH, 1400 rpm), and the textiles were dried in a laundry dryer. By repeated washing (30° C., 16° dH, 1400 rpm, 120 min) and storage under standardized conditions of the prepared laundry in the prepared domestic washing machine, typical domestic washing conditions were simulated and thus corresponding biosoil formation was induced on the treated textiles. After the last wash cycle, the damp laundry and the dried laundry were each sensorially assessed by a trained expert panel and a trained consumer panel.

TABLE 3
FRAGRANCE INTENSITY (EXPERT PANEL, SCALE
1-10, 1 = LOW, 10 = STRONG;
DIFFERENCE ≥1.0 = SIGNIFICANT)

| | V1 | V2 | E1 |
|---|---|---|---|
| Fragrance intensity of damp laundry | 1.50 | 2.50 | 6.50 |
| Fragrance intensity of dry laundry | 2.00 | 2.00 | 3.00 |

TABLE 4
MALODOR (EXPERT PANEL, SCALE 1-10, 1 = LOW,
10 = STRONG; DIFFERENCE ≥1.0 = SIGNIFICANT)

| | V1 | V2 | E1 |
|---|---|---|---|
| Malodor of damp laundry | 6.75 | 5.00 | 1.50 |
| Malodor of dry laundry | 4.00 | 3.00 | 2.00 |

TABLE 5
MALODOR (SCALE 0-3, 0 = NONE, 3 = STRONG;
DIFFERENCE ≥0.3 = SIGNIFICANT)

| | V1 | V2 | E1 |
|---|---|---|---|
| Malodor of damp laundry (consumer panel) | 2.25 | 2.50 | 0.50 |
| Malodor of dry laundry (consumer panel) | 1.20 | 1.00 | 0.66 |
| Malodor of dry laundry (expert panel) | 1.50 | 1.00 | 0.00 |

The results show that the dispersin according to the invention significantly reduces the malodor on damp and dry textiles and also significantly increases the fragrance intensity.

The combination according to the invention of dispersin and fragrance leads to a surprising improvement in the odor perception of damp and dry textiles.

### Example 2: Example Formulations

The present combination according to the invention of dispersin and fragrance can be used in various washing and cleaning compositions and realize its effect.

TABLE 6
LIQUID WASHING AGENTS

| | Wt. % of active substance | | |
|---|---|---|---|
| | in the formulation | | |
| Chemical name | A | B | C |
| Boric acid | 0.6 | 0.6 | 0.6 |
| Citric acid | 0.3 | 0.3 | 0.3 |
| FAEOS | 3.9 | 3.9 | 3.9 |
| FAEO | 4.3 | 4.3 | 4.3 |
| LAS | 3.5 | 3.5 | 3.5 |
| Soap (palm kernel oil) | 0.6 | 0.6 | 0.6 |
| NaOH | 0.8 | 0.8 | 0.8 |
| 1,2-propanediol | 0 | 0 | 0 |
| Glycerol | 0.6 | 0.6 | 0.6 |
| DTPMP | 0.3 | 0 | 0.13 |
| GLDA | 0 | 0.3 | 0.13 |
| Dispersin | 0.3 | 0.3 | 0.3 |
| Fragrance(s) | 0.5 | 0.5 | 0.5 |
| Demineralized water | up to 100 | up to 100 | up to 100 |
| DTI, SRP, other enzymes, | minors | minors | minors |
| defoamers, etc. | | | |

Viscosity [mPas] = 250-550;
Density [g/cm³] = 1.03-1.04;
pH = 8.2-8.6

TABLE 7
LIQUID WASHING AGENTS

| | Wt. % of active substance in the formulation | | | | | |
|---|---|---|---|---|---|---|
| Chemical name | A | B | C | D | E | F |
| Demineralized water | Remainder | Remainder | Remainder | Remainder | Remainder | Remainder |
| LAS | 5.5 | 20 | 15.0 | 5.5 | 21.7 | 23.5 |
| FAEOS | 7.0 | | 5.0 | | | |
| Palm kernel oleic acid | 3.0 | 8.0 | | | 7.0 | 7.4 |
| FAEO | 5.5 | | 8.0 | | | |
| C_{13/15} oxo alcohol, 8EO | | 25 | | | | |
| C₁₂₋₁₈ fatty alcohol ethoxylate, 7EO | | | | | 22.4 | 23.4 |
| Alkyl polyglycoside | | | 4.0 | | | |
| Non-ionic surfactants | | | | 3.1 | | |
| Soap | | | 1.0 | 0.5 | | |
| HEDP | 0.5 | | | | | |
| DTPMPA 7Na | | 1.0 | 1.0 | 0.2 | 0.5 | 1.7 |
| Citric acid | 2.5 | | 3.0 | 0.23 | | |
| NaOH | 3.0 | | | 0.7 | | |
| Glycerol | 3.0 | 5.0 | | 0.5 | 9.4 | 10.2 |
| Ethanol | 1.5 | 3.0 | | | | 3.2 |
| 1,2-propanediol | | 10.0 | 12.0 | | 5.0 | 5.6 |
| Monoethanolamine | | 6.0 | 7.0 | | 6.0 | 6.1 |
| Boric acid | 1.0 | | 1.0 | 0.5 | | |
| Polyalkoxylated alkanolamine | | | | | 4.5 | |
| Ethoxylated polyethyleneimine | | | | | 4.5 | 3.0 |
| Dispersin | 0.26 | 0.26 | 0.26 | 0.26 | 0.26 | 0.26 |
| Fragrance(s) | 0.5 | 0.5 | 0.4 | 0.3 | 0.4 | 0.25 |
| DTI, SRP, other enzymes, | minors | minors | minors | minors | minors | minors |
| defoamers, etc. | | | | | | |

TABLE 8
SOLID WASHING AGENTS

| | Wt. % of active substance | | |
|---|---|---|---|
| | in the formulation | | |
| Chemical name | A | B | C |
| LAS | 12.2 | 12.0 | 10.1 |
| Sodium fatty alcohol sulphate, C₁₂₋₁₈ | 4.2 | | |
| Fatty alcohol, C₁₂₋₁₈, 7 EO | 4.1 | 2.3 | 1.5 |
| Soaps | 0.4 | | |
| Citrate | 2.0 | | |
| Sodium carbonate | 2.4 | 17.9 | 25.1 |
| Builders | 23.0 | 7.0 | 7.6 |
| Phosphonate | 1.2 | 1.1 | 1.2 |
| Polyacrylate | 0.12 | 2.8 | 3.0 |
| Carboxymethylcellulose | 2.3 | 2.0 | 1.1 |
| 2Na2 carbonate 3 H2O2 | 18.5 | 15.8 | |
| TAED | 10.9 | 3.5 | |
| Fragrance(s) | 0.5 | 0.3 | 0.4 |
| Dispersin | 0.3 | 0.3 | 0.3 |
| Sodium sulfate, foam inhibitor, | Remainder | Remainder | Remainder |
| optical brighteners, fragrances, | | | |
| other enzymes | | | |

TABLE 9
HAND DISHWASHING AGENT

| | Wt. % of active substance |
|---|---|
| Chemical name | in the formulation |
| Demineralized water | Remainder |
| FAEOS | 8.8 |
| Cocoamidopropyl betaine | 1.2 |
| Calcium lactate | 1.0 |
| Amylase (Stainzyme 12L) | 0.8 |
| Perfume | 0.2 |
| Dye | 0.01 |
| Salts | 2.0 |
| Dispersin | 0.1 |
| pH | 8.0 |

TABLE 10
TWO-PHASE DISHWASHING AGENT

| Powder phase (phase A) | | |
|---|---|---|
| Active substance content in wt. % | | |
| (unless otherwise stated), based on | | |
| the total weight of the powder phase | A1 | A2 |
| Sodium percarbonate | 13.0 | 15.0 |
| Non-ionic surfactants | 4.0 | 4.0 |
| Sulfonic acid group-containing polymer | 4.0 | 4.0 |
| HEDP (sodium salt) | 6.0 | 6.0 |
| Sodium carbonate (incl. sodium hydrogen | 24.0 | 28.0 |
| carbonate) | | |
| MGDA (trisodium salt) | 0 | 0 |
| Phyllosilicate (SKS 6 powder) | 4.0 | 4.0 |
| Sodium citrate (calculated as anhydrous | 21.0 | 21.0 |
| sodium citrate) | | |
| TAED | 2.1 | 2.1 |
| Zinc acetate | 0.15 | 0.15 |
| Amylase (Stainzyme ® Plus 24 Evity T; | 1.5 | 1.5 |
| indication as wt. % based on the amount | | |
| of preparation used, t.q.) | | |
| Protease (Unity 1000; total active | 40 mg/job | 40 mg/job |
| protein) | | |
| Dispersin (t.q.) | 0.26 | 0.26 |
| Silver protection (cysteine) | 0.15 | 0.15 |
| Misc (perfume, dyes, preservatives, | Add 100 | Add 100 |
| fillers e.g., sodium sulfate, bleach | | |
| catalyst (MnTACN)) | | |

| Gel phase (phase B) | | |
|---|---|---|
| Active substance content in wt. % | | |
| (unless otherwise stated), based on | | |
| the total weight of the gel phase | B1 | B2 |
| Polymer comprising acrylic acid-containing | 10.0 | 11.0 |
| and amidopropyl sulfonic acid-containing | | |
| monomers | | |
| Glycerol | 27.0 | 25.0 |
| 1,3-propanediol | 30.0 | 30.0 |
| PEG 400 | 15.0 | 17.0 |
| PVOH (POVAL 4-88) | 15.0 | 14.0 |
| Misc (inter alia, process auxiliaries, | Add 100 | Add 100 |
| pH adjusters, perfume, dye) | | |
| Gelling time / min | less than 1 | less than 1 |

The phases A1 or A2 and the phases B1 or B2 can be combined with one another as desired. Total weight of both phases in a single portion of 18.5 g.

1. A washing and cleaning agent comprising:
(A) at least one dispersin, wherein the dispersin:
exhibits β-1,6-N-acetylglucosaminidase activity;
comprises an amino acid sequence identical to the amino acid sequence indicated in SEQ ID NO:1 over the entire length thereof by at least 80%;
wherein, based on the numbering according to SEQ ID NO:1, at least one amino acid substitution and/or deletion is present at at least one of the positions corresponding to positions 2, 3, 12, 15, 17, 18, 19, 22, 23, 24, 25, 26, 30, 32, 34, 43, 44, 45, 49, 52, 54, 56, 57, 58, 59, 60, 62, 63, 67, 68, 71, 72, 74, 77, 79, 80, 81, 82, 90, 99, 100, 103, 104, 105, 106, 107, 110, 111, 113, 114, 116, 117, 118, 119, 120, 122, 123, 124, 125, 126, 127, 128, 131, 135, 138, 139, 140, 142, 145, 147, 148, 149, 150, 151, 152, 163, 164, 167, 168, 170, 171, 173, 174, 175, 177, 178, 179, 181, 185, 186, 187, 188, 189, 199, 200, 203, 204, 205, 207, 208, 210, 215, 217, 218, 221, 222, 224, 225, 227, 230, 232, 233, 234, 235, 237, 244, 249, 251, 252, 253, 254, 256, 260, 261, 262, 263, 264, 265, 267, 268, 270, 271, 272, 273, 274, 276, 278, 279, 280, 281, 282, 283, 284, 287, 288, 290, 291, 296, 300, 301, 303, 304, 305, 306, 308, 309, 312, 314, 315, 319, 321 and 323; and,
wherein the dispersion is present in an amount of active protein of 0.00001 to 5 wt. %, based on the total weight of the washing and cleaning agent;
(B) at least one fragrance, in an amount of 0.01 to 5 wt based on the total weight of the agent, and
(C) at least one ingredient, in an amount of 0.01 to 99.9 wt. %, based on the total weight of the agent.

2. The washing and cleaning agent according to claim 1, wherein the at least one amino acid substitution and/or deletion is selected from the group consisting of: D2A, D2L, D2N, D2R, D2V, D2W, Q3F, Q3I, Q3L, Q3M, Q3P, Q3V, Q3Y, Q3T, S12A, H15F, H15Y, T17W, T17C, T17E, T17F, T17M, T17R, T17V, V18L, E19D, E19N, E19P, K22A, K22M, K22V, S23C, S23E, S23I, S23L, S23R, S23T, S23V, V25R, D26M, Y30*, Y30D, Y30L, Y30M, Y30N, Y30R, Y30T, Y30V, G32L, G32M, G32R, N43*, N43H, N43L, E44*, N45D, N45L, N45V, A49W, A49Y, Y52*, Y52M, G54L, G54M, G54N, S56T, S56W, S57W, E58N, N59A, N59C, N59D, N59E, N59F, N59M, N59R, N59V, N59W, T60V, N62C, N62D, N62H, N62Q, N62W, T63C, T63D, T63L, T63N, T63R, T63V, K67A, K67L, N68L, N68Q, L71H, L71N, L71R, L71V, L71W, S72*, S72C, S72D, S72E, S72F, S72G, S72I, S72M, S72N, S72R, S72T, S72Y, I74L, S77A, D79V, K80*, K80E, K80H, K80L, K80N, K80Q, K80V, K80W, D81A, D81G, D81L, D81R, D81S, D81T, D81V, D81W, I82V, L90F, E99Q, E99R, L100S, K103A, K103R, K104N, K104W, D105N, V106A, V106D, V106E, V106H, V106K, V106L, V106M, V106N, V106Q, V106W, V106Y, K107A, K107C, K107L, K107M, K107T, K107V, K107W, N110M, N110R, N110V, D111A, D111E, D111M, D111N, D111Q, D111R, D111V, D111W, V113T, T114C, T114S, Y116D, Y116N, Y116R, S117*, S117D, S117H, S117N, S117P, E118*, E118A, E118D, E118G, E118L, E119G, E119W, T120I, T120L, T120M, T120V, T120W, D122*, D122H, D122R, Y123W, Y124C, Y124I, Y124K, Y124L, Y124M, Y124Q, Y124R, Y124T, Y124V, Y124W, D125C, D125G, D125K, D125Q, D125R, N126V, R127D, R127H, R127K, R127L, R127M, R127Q, R127W, V128C, V128L, V128T, D131V, Q135*, Q135A, Q135D, Q135E, Q135K, Q135M, Q135Y, D138K, D138L, D138M, D138Q, D138R, D138S, D138V, D138W, E139W, D142R, D142W, Y145*, Y145H, Y145L, Y145N, Y145V, P147A, P147C, P147D, P147F, P147G, P147L, P147M, P147R, P147S, P147T, P147V, K148A, K148D, K148L, K148V, F149L, F149M, F149N, E150D, E150H, E150K, E150L, E150M, E150N, E150R, E150V, E150W, E150Y, G151A, G151C, G151D, G151L, G151N, G151P, G151S, G151W, K152D, K152L, K152R, G164D, G164E, G164H, G164S, G164V, V167D, V167E, V167L, V167P, V167Q, V167R, V167W, H168N, L170A, L170D, L170E, L170F, L170H, L170K, L170M, L170N, L170P, L170Q, L170R, L170S, L170V, L170W, L170Y, D171A, D171C, D171E, D171K, D171L, D171M, D171Q, D171R, D171V, D171W, D171Y, I173C, D174H, D174M, D174N, D174V, D174W, F175Y, N177M, Q178*, Q178A, Q178K, Q178R, Q178W, I179T, S181C, S181D, S181F, S181G, S181N, S181P, S181Q, S181T, S181V, S181W, E185M, E185R, E185V, E185W, S186D, S186E, S186H, S186I, S186K, S186L, S186M, S186N, S186Q, S186R, S186V, S186W, K187C, K187D, K187G, K187R, K187S, K187V, K187W, Y188P, E189L, E189V, E189W, S199C, S199L, S199M, S199Y, E200D, E200F, E200K, E200L, E200M, E200N, E200R, E200W, A203C, A203D, A203E, A203G, A203L, A203M, A203P, A203R, A203S, A203T, A203V, A203W, N204L, N204M, N204V, N204W, N204Y, L205I, D207A, D207C, D207E, D207G, D207N, D207Q, D207S, D207V, D207W, S208A, S208C, S208D, S208G, S208L, S208Q, S208T, S208V, S208W, S210T, Q215R, Q215M, Q215L, Q215*, S217V, T218A, T218L, T218Q, T218R, T218V, G222D, E224A, E224P, N227A, N227Q, N227R, N227S, N227T, N227K, D230*, D230R, D230T, D230W, E232D, E232V, N233H, N233Q, N233R, N233W, W234R, G235W, G235A, G235E, G235F, G235H, G235I, G235L, G235M, G235N, G235P, G235S, G235V, S237C, S237G, S237M, S237N, S237W, S237Y, Y244C, Y244E, Y244M, L249H, L249K, L249Q, L249R, L249W, L249Y, S251L, S251N, S251R, S251W, N252P, N252C, G253D, G253W, F254I, F254L, F254M, F254Y, Q256E, Q256R, N260*, N260A, N260C, N260E, N260I, N260K, N260L, N260M, N260Q, N260R, N260V, N260W, N260Y, E261*, E261A, E261D, E261R, E261W, Q262*, Q262F, Q262H, Q262W, Q262Y, M263K, M263L, M263Q, D264*, D264C, D264E, Y265F, N267S, N267T, W268C, W268E, W268M, W268R, Y270F, A271D, A271G, H272D, H272I, Y281P, Y282E, Y282N, Y281R, H272W, N273W, K274R, K274A, K274H, F276A, F276C, F276K, F276N, F276G, F276L, F276M, F276P, F276S, F276V, F276W, I278A, I278K, I278N, I278Q, I278V, S279C, S279D, S279E, S279G, S279N, D280C, D280E, Y281*, Y281A, Y281C, Y281H, Y281K, Y281N, Y281P, Y282E, Y282N, H283I, A284I, A284L, A284N, A284P, A284T, A284V, T287N, S288D, S288K, S288N, V290I, K291L, K291R, K291V, T296C, E300A, E300D, H301C, H301N, H301R, T303A, T303C, T303G, T303K, T303Q, T303R, T303W, D304C, D304M, L305M, L305N, S306C, K308A, K308D, K308G, K308I, K308L, K308Q, K308S, K308T, K308V, K308Y, K309A, K309C, K309D, K309H, K309L, K309M, K309N, K309Q, K309S, K309T, K309I, K312A, K312L, K312M, K312N, K312Q, K312S, K312W, E314I, E314L, E314V, L315I, L315V, R319A, N323R, and combinations thereof.

3. The washing and cleaning agent according to claim 1, wherein the fragrance is selected from the group consisting of: α-isomethyl ionone, benzyl salicylate, citronellol, coumarin, hexyl cinnamal, linalool, pentanoic acid, 2-methylethyl ester, octanal, benzyl acetate, 1,6-octadien-3-ol, 3,7-dimethyl-3-acetate, cyclohexanol, 2-(1,1-dimethylethyl)-1-acetate, δ-damascone, β-ionone, verdyl acetate, dodecanal, hexyl cinnamaldehyde, cyclopentadecanolide, benzene acetic acid, 2-phenylethyl ester, amyl salicylate, β-caryophyllene, ethyl undecylenate, geranyl anthranilate, α-iron, β-phenylethylbenzoate, α-santalol, cedrol, cedryl acetate, cedryl formate, cyclohexyl salicyate, γ-dodecalactone, p-phenylethylphenylacetate, 2-methyl-6-methylene-7-octen-2-ol (dihydroderivatives), 3A,4,5,6,7,7A-hexahydro-4,7-methano-1H-inden-5(or 6)-ylacetate, oxydipropanol, 1-(1,2,3,4,5,6,7,8-octahydro-2,3,8,8-tetramethyl-2-naphthyl)ethan-1-one, 2-phenylethanol, exo-1,7,7-trimethylbicyclo-[2.2.1]-hept-2-yl acetate, 3,7-dimethyl-3-octanol, 4-(2,6,6-trimethylcyclohex-1-en-1-yl)but-3-en-2-one, hedione, 2-ethyl-4-(2,2,3-trimethyl-3-cyclopenten-1-yl)-2-buten-1-ol, p-menth-1-en-8-yl acetate, orange oil (sweet), hexyl salicylate, (R)-p-mentha-1,8-diene, 3a,4,5,6,7,7a-hexahydro-4,7-methano-1H-indenylpropionate, 3-(dodecylthio)-1-(2,6,6-trimethyl-3-cyclohexen-1-yl)-1-butanone, 2-methylundecanal, 3-p-cumenyl-2-methylpropionaldehyde, allylheptanoate, nerol, pin-2(3)-ene, diphenyl ether, methyl 2-naphthyl ether, methyl benzoate, 5-heptyldihydro-2(3H)-furanone, isopropyl ester myristic acid, γ-decalactone, ethyl 2-methylvalerate, 4-methyl-3-decen-5-ol, cineole, tetrahydro-3-pentyl-2H-pyran-4-yl acetate, methyl anthranilate, dimethylcyclohex-3-en-1-carbaldehyde, patchouli extract, 3,7-dimethyl-2,6-nonadienenitrile, decanal, patchouli oil, [2-(1-propoxyethoxy)ethyl]benzene, 1-(2,6,6-trimethyl-3-cyclohexen-1-yl)-2-buten-1-one, decene (hydroformylation products), pin-2(10)-ene, DL-borneol, eugenol, ethyl-2-methylbutyrate, 1,3-dimethyl-3-butenyl-2-methylpropionate, 4-methylanisole, 3-phenyl-2-propenoic acid methyl ester, patchouli alcohol, 9-undecenal, borna-2-one, 2-heptylcyclopentanone, (Z)-3-hexen-1-ol acetate, 4′-methylacetophenone, 1-(5,5-dimethyl-1-cyclohexen-1-yl)pent-4-en-1-one, 2-aminoethanol, 3,3-dimethyl-5-(2,2,3-trimethyl-3-cyclopenten-1-yl)-4-penten-2-ol, (1S-cis)-1,2,3,4,5,6,7,8-octahydro-7-isopropylidene-1,4-dimethylazulene, acetophenone, anethole, 3-methyl-5-(2,2,3-trimethyl-3-cyclopenten-1-yl)-4-penten-2-ol, trans-hex-2-enal, 2-undecanone, 4-(4-hydroxyphenyl)butan-2-one, phenylethyl methyl ether, methyl salicylate, undecanal, tetrahydro-4-methyl-2-(2-methyl-1-propenyl)-2H-pyran, 2,6-dimethyl-5-heptenal, acetaldehyde ethyl phenyl ethyl acetal, b-4-dimethyl-3-cyclohexene-1-propanal, peppermint oil, d-p-mentha-1(6),8-dien-2-one, and mixtures thereof.

4. The washing and cleaning agent according to claim 1, wherein
the dispersin and the fragrance interact synergistically and lead to an increased fragrance intensity of the fragrance contained in the agent on a textile or surface treated with the agent when the fragrance intensity is determined as described in Example 1, and/or
the dispersin and the fragrance interact synergistically and lead to a reduced malodor of a textile or surface treated with the agent when the malodor is determined as described in Example 1.

5. The washing and cleaning agent according to claim 1, wherein the washing and cleaning agent ingredient is selected from the group consisting of: surfactants, builders, complexing agents, polymers, glass corrosion inhibitors, corrosion inhibitors, bleaching agents such as peroxygen compounds, bleach activators or bleach catalysts, water-miscible organic solvents, enzyme stabilizers, sequestering agents, electrolytes, pH regulators, and/or other auxiliaries such as optical brighteners, graying inhibitors, dye transfer inhibitors, foam regulators, and dyes and fragrances, and combinations thereof.

6. The washing and cleaning agent according to claim 1, wherein
the agent is substantially free of boron-containing compounds; and/or
the agent has a pH in a range of approximately 6 to approximately 11, in a 1 wt. % solution in deionized water at 20° C.

7. A method, comprising:
using in at least one method step the washing and cleaning agent of claim 1,
wherein the method is carried out in a temperature range from approximately 20° C. to approximately 60° C.

8. The method of claim 7 wherein the method step includes: cleaning textiles and/or hard surfaces, dishes, and deep cleaning, in a temperature range of approximately 20° C. to approximately 60° C.

9. The method of claim 7 wherein the dispersin is used for reducing the malodor of a textile or a surface treated with the agent as described in Example 1, in a temperature range of approximately 20° C. to approximately 60° C.

10. A use of a dispersin in a washing and/or cleaning agent according to claim 1 for increasing the fragrance intensity of the fragrance contained in the agent on a textile or a surface which has been treated with the agent, when determined as described in Example 1, preferable in a temperature range of approximately 20° C. to approximately 60° C.
